(19) United States
(12) Patent Application Publication
(10) Pub. No.: US 2023/0390361 A1
(43) Pub. Date: Dec. 7, 2023

(54) IL-2/IL-15R-BETA-GAMMA AGONIST FOR TREATING SQUAMOUS CELL CARCINOMA

(71) Applicant: Cytune Pharma, Nantes (FR)

(72) Inventors: Stefano FERRARA, Allschwil (CH); Ulrich MOEBIUS, Gauting-Unterbrunn (DE); David BÉCHARD, Saint-Etienne de Montluc (FR); Irena ADKINS, Prezletice (CZ); Nada PODZIMKOVA, Jindrichuv Hradec (CZ)

(21) Appl. No.: 18/033,773

(22) PCT Filed: Oct. 26, 2021
(86) PCT No.: PCT/EP2021/079636

§ 371 Date: Apr. 25, 2023

(30) Foreign Application Priority Data
Oct. 26, 2020 (EP) 20203908.7

(51) Int. Cl. A61K 38/20; A61P 35/00

(52) U.S. Cl. CPC A61K 38/2086; A61P 35/00

(57) ABSTRACT

The present invention relates to an interleutkin-2/interleutkin-15 receptor βγ (IL-2/IL-15Rβγ) agonist for use in the treatment of squamous cell carcinoma. Further provided are dosing schemes for treating patients with squamous cell carcinoma with an IL-2/IL-15Rβγ agonist.

### BACKGROUND OF THE INVENTION

Despite recent advances in the treatment of cancer and infectious diseases, there is still an unmet medical need for more effective and well-tolerated treatments. Immunotherapies, i.e. treatments that make use of the body's own immune system to help fighting the disease, aim at harnessing the power of the immune system to kill malignant tumor cells or infected cells, while leaving healthy tissues intact. Whereas the immune system has an inherent ability to find and eliminate malignancies, tumors and persistent infections have developed mechanisms to escape immune surveillance (Robinson and Schluns 2017). The potential reasons for immune tolerance include failed innate immune activation, the involvement of dense stroma as a physical barrier, and a possible contribution of immune suppressive oncogene pathways (Gajewski et al. 2013). One group of immunotherapies with some clinical success are cytokine treatments, more specifically interleukin 2 (IL-2), commercially available as aldesleukin/PROLEUKIN® (Prometheus Laboratories Inc.) and interleukin 15 (IL-15) therapies known to activate both the innate immune response through NK cells and the adaptive immune response through CD8⁺ T cells (Steel et al. 2012, Conlon et al. 2019). While impressive tumor regression was observed with IL-2 therapy, responses are limited to small percentages of patients and carry with it a high level of even life-threatening toxicity. Further, IL-2 displayed not only immune-enhancing but also immune-suppressive activities through the induction of activation-induced cell death of T cells and the expansion of immunosuppressive regulatory T cells (T_{regs}). (Robinson and Schluns 2017)

Both IL-2 and IL-15 act through heterotrimeric receptors having α, β and γ subunits, whereas they share the common gamma-chain receptor (γ_{c} or γ)—also shared with IL-4, IL-7, IL-9 and IL-21-and the IL-2/IL-15Rβ (also known as IL-2Rβ, CD122). As a third subunit, the heterotrimeric receptors contain a specific subunit for IL-2 or IL-15, i.e. the IL-2Rα (CD25) or the IL-15Rα (CD215). Downstream, IL-2 and IL-15 heterotrimeric receptors share JAK1 (Janus kinase 1), JAK 3, and STAT3/5 (signal transducer and activator of transcription 3 and 5) molecules for intracellular signaling leading to similar functions, but both cytokines also have distinct roles as reviewed in Waldmann (2015, see e.g. table 1) and Conlon (2019). Accordingly, the activation of different heterotrimeric receptors by binding of IL-2, IL-15 or derivatives thereof potentially leads to a specific modulation of the immune system and potential side effects. Recently, the novel compounds were designed aiming at specifically targeting the activation of NK cells and CD8⁺ T cells.

These are compounds targeting the mid-affinity IL-2/IL-15Rβγ, i.e. the receptor consisting of the IL-2/IL-15Rβ and the γ_{c} subunits, which is expressed on NK cells, CD8⁺ T cells, NKT cells and γδ T cells. This is critical for safe and potent immune stimulation mediated by IL-15 trans-presentation, whereas the designed compounds SO-C101 (SOT101, RLI-15), nogapendekin-alfa/inbakicept (ALT-803) and hetIL-15 already contain (part of) the IL-15Rα subunit and therefore simulate transpresentation of the α subunit by antigen presenting cells. SO-C101 binds to the mid-affinity IL-15Rβγ only, as it comprises the covalently attached sushi+ domain of IL-15Rα. In turn, SO-C101 does bind neither to IL-15Rα nor to IL-2Rα. Similarly, ALT-803 and hetIL-15 carry an IL-15Rα sushi domain or the soluble IL-15Rα, respectively, and therefore bind to the mid-affinity IL-15Rβγ receptor. However, due to their non-covalent binding there is a chance that the complex dissociates in vivo and thereby the dissociated fraction of the applied complex further exerts other binding (see below). Probability for dissociation is likely higher for ALT-803 vs. hetIL-15, as ALT-803 only comprises the sushi domain of IL-15Rα, which is known to mediate only partial binding to IL-15, whereas the sushi+domain is required for full binding (Wei et al. 2001).

Another example of targeting mid-affinity IL-2/IL-15Rβ receptors is NKTR-214, whose hydrolysation to its most active 1-PEG-IL-2 state generates a species whose location of PEG chains at the IL-2/IL-2Rα interface interferes with binding to the high-affinity IL-2Rα, while leaving binding to the mid-affinity IL-2/IL-15Rβ unperturbed (Charych et al. 2016). Further, the mutant IL-2 IL2v with abolished binding to the IL-2Rα subunit is an example of this class of compounds (Klein et al. 2013, Bacac et al. 2016). Also, the IL-2/IL-2Rα fusion protein nemvaleukin alfa (ALKS 4230) comprising a circularly permutated (to avoid interaction of the linker with the β and γ receptor chains) IL-2 with the extracellular domain of IL-2Rα selectively targets the βγ receptor as the α-binding side is already occupied by the IL-2Rα fusion component (Lopes et al. 2020). The targeting of the mid-affinity IL-2/IL-15Rβγ receptors avoid liabilities associated with targeting the high-affinity IL-2 and IL-15 receptors such as T regulatory cells (T_{regs}) activation induced by IL-2 or vascular leakage syndrome which can be induced by high concentrations of soluble IL-2 or IL-15.

This is due to the fact that the IL-2Rαβγ high affinity receptor is additionally expressed on CD4⁺ T_{regs} and vascular endothelium and is activated by IL-2 cis-presentation. Therefore, compounds targeting (also) the high-affinity IL-2Rαβγ potentially lead to T_{reg} expansion and vascular leak syndrome (VLS), as observed for native IL-2 or soluble IL-15 (Conlon et al. 2019). Potentially VLS can be also caused by the de-PEGylated NKTR-214. De-PEGylated NKT2-214 has however a short half-life and it needs to be seen in the clinical development whether at all or to which extent this side-effect plays a role.

The high-affinity IL-15Rαβγ receptors activated by IL-15 cis-presentation are constitutively expressed in T cell leukemia and upregulated on inflammatory NK cells, inflammatory CD8⁺ T cells and Fibroblast-like synoviocytes (Kurowska et al. 2002, Perdreau et al. 2010), i.e. these cells also express the IL-15Rα subunit. Such activation should be avoided because of the IL-15 cis-presentation on these cells is involved in the development of T cell leukemia and exacerbation of the immune response, potentially triggering autoimmune diseases. Similarly, the high-affinity IL-15Rαβγ receptor is expressed on vascular endothelium and soluble IL-15 can also induce VLS. IL-15/IL-15Rα complexes do not bind to this high-affinity receptor as they already carry at least the sushi domain of the IL-15Rα, which sterically hinders the binding to the heterotrimeric IL-15Rαβγ receptor. These side effects triggered via engagement of high affinity IL-15Rαβγ receptors are triggered by native IL-15, but also by non-covalent IL-15/IL-15Rα complexes such as ALT-803 and hetIL-15, if disintegration of the complexes occurs in vivo.

Finally, the high-affinity IL-15Rα is constitutively expressed on myeloid cells, macrophages, B cells and neutrophils (Chenoweth et al. 2012) and may be activated by native IL-15 and again by non-covalent IL-15/IL-15Rα complexes such as ALT-803 and hetIL-15, if disintegration of the complexes occurs in vivo.

In summary, IL-15 has similar immune enhancing properties as IL-2, but it is believed to not share the immune-suppressive activities like activation of T_{reg} cells and does not cause VLS in the clinic (Robinson and Schluns 2017), whereas drawbacks of IL-15 treatment include its short in vivo half-life and its reliance on trans-presentation by other cell types (Robinson and Schluns 2017). This leads to the development of engineered IL-2/IL-15Rβγ agonists, some of them recently entered clinical development.

Although high-dose IL-2 treatment is approved in renal cell carcinoma and metastatic melanoma (at 600,000 IU/kg administered by i.v. bolus over 15 min every 8 hours for a maximum of 14 doses, following 9 days of rest before the regimen is repeated if tolerated by the patient), IL-2 still continues to be investigated in order to define a lower-dose schedule that provides sufficient immune activation with a better tolerated safety profile, e.g. by infusion over 90 days at low-dose expand NK cells with intermediate pulses of IL-2 to provide activation of an expanded NK cell pool and many other low-dose i.v. or s.c. treatments usually given in combination with other immunotherapeutics have been assessed but with inconclusive results (Conlon et al. 2019). Low dose s.c. regimens (1-30 million IU/m²/d) have been investigated because they may reduce toxicity but compromise efficacy (Fyfe et al. 1995) but preferentially activate T_{regs}. Therefore, low dose IL-2 is used in immunosuppressive treatments (Rosenzwajg et al. 2019).

Accordingly, administration, dosing and dosing schedules of the engineered IL-2/IL-15Rβγ agonists will be key for their clinical success, which is driven by multiple factors, for example related to efficacy, side effects, patient compliance and convenience e.g. in combinations with other drugs.

Recently, pharmacokinetics and pharmacodynamics of hetIL-15 in rhesus macaques were published (Bergamaschi et al. 2018). hetIL-15 was dosed s.c. at fixed doses of 0.5, 5 or 50 μg/kg in dosing cycles with administration on days 1, 3, 5, 8, 10 and 12 (cycle 1) and on days 29, 31, 33, 36, 38 and 40 (dosing cycle 2). Further, monkeys were dosed with a doubling step-dose regimen with injections on days 1, 3, 5, 8, 10 and 12 at doses of 2, 4, 8, 16, 32 and 64 μg/kg. Iv. administration leads to a peak of IL-15 plasma levels at 10 min after injection with a half-life of about 1.5 h, whereas s.c. administration of hetIL-15 resulted in a T, of about 12 h. It was shown that both AUC and Cₘₐₓ were reduced between day 1 and 40 upon treatment with a fixed dose s.c., 2-fold and 4-fold at fixed dose of 5 μg/kg, and even 9-fold and 8-fold at a fixed dose of 50 μg/kg. The authors conclude that “the consumption of the administered hetIL-15 progressively increased during the treatment cycle, reflecting an increase in the pool of cells responding to IL-15” and that “the fixed-dose regimen provided an excess of IL-15 early in the 2-week cycle but not enough cytokine later in the treatment cycle”. The authors therefore continued with an administration scheme consisting of 6 progressively doubling doses from 2 to 64 μg/kg of hetIL-15 over the course of two weeks, leading to a progressive increase in systemic exposure and comparable trough levels, overall interpreted to better match the increasing IL-15 need by the expanding pool of target cells during treatment. With respect to the proliferation of CD8⁺ T cells, the authors observed with the fixed-dose regimens a decline at day 15 for proliferating Ki67⁺CD8⁺ T cells, whereas macaques treated with the step-dose regiment showed high and comparable CD8⁺ T cell proliferation on day 8 and 15.

Most of the designed IL-2/IL-15Rβγ agonists aim for increasing their in vivo half-life either by fusing the IL-15, IL-2 or variant thereof to another protein, e.g. to the soluble IL-15Rα (hetIL-15, where the complexation with the receptor goes along with a considerable extension of the half-life), to add an Fc part of an antibody to the complex (ALT-803) or IL-15/IL-15Rα Fc fusions (P22339) disclosed in U.S. Pat. No. 10,206,980 and IL15/IL15Rα heterodimeric Fc-fusions with extended half-lives (Bernett et al. 2017) (WO 2014/145806), to a non-binding IgG (IgG-IL2v) or to an albumin binding domain (see WO 2018/151868A2). Other examples of IL-2/IL-15Rβγ agonists are CT101-IL2 (Ghasemi et al. 2016, Lazear et al. 2017), PEGylated IL-2 molecules like and NKTR-214 (Charych et al. 2016) and THOR-924 (Caffaro et al. 2019) (WO 2019/028419, WO 2019/028425), the polymer-coated IL-15 NKTR-255 (Miyazaki et al. 2018), NL-201/NEO-201 (Silva et al. 2019), RGD-targeted IL-15/IL-15Rα Fc complex (US 2019/0092830), RTX-240 by Rubius Therapeutics (red blood cells expressing an IL-15/IL-15Rα fusion protein, WO 2019/173798), and THOR-707 (Joseph et al. 2019). Further, targeted IL-2/IL-15Rβγ agonists, where the agonist is fused to a binding molecule targeting specific cells, e.g. tumor, tumor-microenvironment or immune cells, have an increased in vivo half-life (RG7813, RG7461, immunocytokines of WO 2012/175222A1, modulokines of WO 2015/018528A1 and KD033 by Kadmon, WO 2015/109124).

Studies indicated that ALT-803 has a 7.5-hour serum half-life in mice (Liu et al. 2018) and 7.2 to 8 h in cynomolgus monkeys (Rhode et al. 2016) compared with <40 minutes observed for IL-15 (Han et al. 2011). In the clinic, ALT-803 was administered i.v. or s.c. in a Phase I dose escalation trial weekly for 4 consecutive weeks, followed by a 2-week rest period for continued monitoring, for two 6-week cycles of therapy starting at 0.3p g/kg up to 20 μg/kg. Results from the trial led to the selection of 20 μg/kg/dose s.c. weekly as the optimal dose and route of delivery for ALT-803 (Margolin et al. 2018).

NKTR-214 is described as a highly “combinable cytokine” dosed more like an antibody than a cytokine due to its long half-life in vivo. Its anticipated dosing schedule in humans is once every 21 days. Yet NKTR-214 provides a mechanism of direct immune stimulation characteristic of cytokines. PEGylation dramatically alters the pharmacokinetics of NKTR-214 compared with IL-2, providing a 500-fold increase in AUC in the tumor compared with an IL-2 equivalent dose. Pharmacokinetics of NKTR-214 were determined after i.v. administration in mice and resulted for the most active species of NKTR-214 (i.e. 2-PEG-IL2, 1-PEG-IL2, free IL2) in a gradually increase, reaching Cₘₐₓ at 16 hours post dose and a decrease with t, of 17.6 hours (Charych et al. 2017). Based on the increased half-life due to PEGylation, NKTR-214 was tested in five dose regimens in combination with nivolumab in NCT02983045 (see wwwclincaltria)
0.006 mg/kg NKTR-214 every 3 weeks (q3w) with 240 mg nivolumab every two weeks (q2w),
0.003 mg/kg NKTR-214 q2w with 240 mg nivolumab q2w,
0.006 mg/kg NKTR-214 q2w with 240 mg nivolumab q2w,
0.006 mg/kg NKTR-214 q3w with 360 mg nivolumab q3w,
0.009 mg/kg NKTR-214 q3w with 360 mg nivolumab q3w.

After completion of the first part of the study it was continued with a dose of 0.006 mg/kg NKTR-214 q3w with 360 mg nivolumab q3w.

Recently, IL-2/IL-15 mimetics have been designed by a computational approach, which is reported to bind to the IL-2Rβγ heterodimer but have no binding site for IL-2Rα (Silva et al. 2019) and therefore also qualify as IL-2/IL-15Rβγ agonists. Due to their small size of about 15 kDa (see supplementary information Figure S13) they are expected to have a rather short in vivo half-life.

Another example of such IL-2 based IL-2/IL-15Rβγ agonist is an IL-2 variant (IL2v) by Roche, which is used in fusion proteins with antibodies. R0687428, an example comprising IL2v, is administered in the clinic i.v.
on days 1, 15, 29, and once in 2 weeks from day 29 onwards with a starting dose of 5 mg and increased subsequently, or in a q3w schedule (see NCT03063762, www.clinicaltrials.gov),
once weekly (qw) with a starting does of 5 mg as monotherapy,
with a starting dose of 5 mg qw in combination with cetuximab and
with a starting dose of 10 mg qw in combination with trastuzumab (see NCT02627274, www.clinicaltrials.gov), or in combination with atezolizumab,
qw for first 4 doses, and once in 2 weeks (q2w) for remaining doses up to maximum 36 months starting with a first dose of 10 mg and 15 mg for the second and following doses,
qw for first 4 doses and q2w for remaining doses up to maximum 36 months with a starting dose of 10 mg and 15 mg for. the second and following doses,
q3w up to max. 36 months with a dose of 10 mg,
qw for 4 weeks followed by q2w with a starting dose of 15 mg and 20 mg from the second administration onward, or
q3w with a dose of 15 mg (see NCT03386721, www.clinicaltrials.gov).

TABLE 1
In vivo half-life of IL-15 and IL-2/IL-15Rβγ agonists

| | T ½ mouse s.c. | | T ½ human | optimized human admin. | |
|---|---|---|---|---|---|
| IL-15 | <40 | min | Tₘₐₓ 4 h after s.c. | s.c. days 1-8 and 22-29, | NCT03388632 |
| (rhIL-15) | | | bolus i.v. T½ = | or | NCT01572493 |
| | | | 2.7 h | i.v. continuous infusion | NCT01021059 |
| | | | | for 5 or 10 consecutive | (Han et al. 2011) |
| | | | | days, or | (Miller et al. 2018) |
| | | | | i.v. daily for 12 | (Conlon et al. 2015) |
| | | | | consecutive days | |
| ALT-803 | 7.5 h for i.v. | | s.c >96 h, but not | 20 μg/kg s.c. qw | (Romee et al. 2018) |
| | versus 7.7 h for | | i.v. | | (Wrangle et al. 2018) |
| | S.C. | | Cₘₐₓ after 6 h, still | | |
| | | | detectable at 24 h | | |
| hetIL-15, | ~12 | h | | 6 progressively | (Bergamaschi et al. 2018) |
| NIZ985 | | | | doubling doses from 2 | (Conlon et al. 2019) |
| | | | | to 64 μg/kg over the | |
| | | | | course of 2 weeks | |
| | | | | 1 μg/kg (3x weekly; 2- | |
| | | | | weeks-on/2-weeks-off) | |
| RLI-15 | 3.5 h (own data) | | approx. 4 h after | s.c. | own data |
| | | | s.c. | | |
| NKT-214 | multiple days | | T½ 20 h, Cₘₐₓ 1-2 | 6 μg/kg i.v. q3w | (Charych et al. 2017) |
| | | | days post dose | | (Bentebibel et al. 2017) |
| NKTR-214 | 17.6 | h | | | (Charych et al. 2017) |

| | | |
|---|---|---|
| most active | | |
| species | | |
| RO687428 | ≥5 mg i.v. qw or q3w | NCT03386721 |

However, already less than 15 min exposure of cells with IL-15 (at 10 ng/ml) expressing the receptor to native IL-15 leads to the maximal level of Stat5 activation and subsequent pharmacodynamic effects (Castro et al. 2011).

In summary, presently IL-2/IL-15Rβγ agonists are dosed in order to achieve a continuous availability of the molecule in the patient, either by continuous infusion of short-lived molecules or by extending drastically the half-life of IL-2/IL-15Rβγ agonists through PEGylation or fusion to Fc fragments or antibodies. This is in line with the common understanding that both the tumor homing and the in vivo anti-tumor activity of NK cells are dependent on the continuous availability of IL-2 or IL-15, whereas if NK cells are not frequently stimulated by IL-15, they rapidly die (Larsen et al. 2014). Further, such therapies focus very much at maximizing the CD8⁺ T-cell expansion, whereas at the same time try to minimize the T_{reg} expansion (Charych et al. 2013).

On the other hand, Frutoso et al. demonstrated in mice that two cycles of injection of IL-15 or IL-15 agonists resulted in a weak or even no expansion of NK cells in vivo in immunocompetent mice, whereas CD44⁺ CD8⁺ T cells were still responsive after a second cycle of stimulation with IL-15 or its agonists (Frutoso et al. 2018). Escalating the dose for the second cycle did not make a marked difference. Furthermore, NK cells extracted from mice after two cycles of stimulation had a lower IFN-γ secretion compared to after one cycle, which was equivalent to that of untreated mice (Frutoso et al. 2018). This phenomenon may be explained by the findings that prolonged stimulation of NK cells with a strong activation signal leads to a preferential accrual of mature NK cells with altered activation and diminished functional capacity (Elpek et al. 2010). Similarly, continuous treatment with IL-15 was described to exhaust human NK cells and this effect was brought into context with the influence of fatty acid oxidation on the activity of NK cells suggesting that induces of fatty acid oxidation have the potential to greatly enhance IL-15 mediated NK cell immunotherapies (Felices et al. 2018).

Despite the growing understanding of the innate and adaptive immunity related to cytokine treatment, the initial single-agent clinical trials with the long awaited IL-15 as monotherapy have not fulfilled the promise of efficacy seen in preclinical experiments, whereas combination trials are still ongoing (Conlon et al. 2019). It is still very much unclear, in which indications the IL-2 and IL-15 agonists/superagonist may actually lead to significant treatment benefits for the patients. Due to the shown efficacy of high dose IL-2 in metastatic melanoma and metastatic renal cell carcinoma and some signs of efficacy of IL-15 in metastatic melanoma (stable disease at best, phase 1, daily bolus infusion) (Conlon et al. 2019) likely due to their known high immunogenicity (Haanen 2013, Prattichizzo et al. 2016), melanoma and renal cell carcinoma are the primary indications were the βγ agonists are tested. Still, Conlon concludes that it is clear from trials that IL-15 to make a major impact in cancer treatment must be administered in combination with agents that already have an action, although inadequate in the treatment of cancer (Conlon et al. 2019). Accordingly, the βγ agonists are broadly tested in combination with immune checkpoint inhibitors (or short: checkpoint inhibitors) or anti-cancer antibodies to increase their antibody-dependent cellular cytotoxicity (ADCC), anti-cancer vaccines or cellular therapies.

Therefore, despite recent advances in understanding the function of the IL-2/IL-15Rβγ agonists, it is still unclear how such IL-2/IL-15Rβγ agonists are optimally dosed and integrated into treatment regimens and which patients beyond those suffering from melanoma and renal cell carcinoma may benefit from the treatment with the βγ agonist as a single agent or in combination with other treatments.

### SUMMARY OF THE INVENTION

The inventors have surprisingly found that an interleukin-2/interleukin-15 receptor βγ (IL-2/IL-15Rβγ) agonist exhibits single agent activity in cancer treatment. Further, they could unexpectedly show anti-tumor activity in a cancer patient refractory to checkpoint inhibitor treatment. The inventors identified that a pulsed cyclic dosing of an IL-2/IL-15Rβγ agonist in primates lead to an optimal activation of NK and CD8⁺ T cells, i.e. that the administration of the IL-2/IL-15Rβγ agonist results in a marked increase of Ki-67⁺ NK cells and CD8⁺ T cells and/or an increase in NK cell and CD8⁺ T cell numbers, which is repeated/maintained during multiple rounds of administration. Such pulsed cyclic dosing schedule showed a very benign safety profile in a first-in-human study (presently still ongoing) and, surprisingly, showed single-agent activity in a patient suffering from late stage, checkpoint-inhibitor refractory skin squamous cell carcinoma. This treatment success opens a new understanding of what IL-2/IL-15Rβγ agonists can achieve and which indications are susceptible to IL-2/IL-15Rβγ agonist treatment.

Accordingly, the present invention provides IL-2/IL-15Rβγ agonist treatment for new tumor indications and patient groups.

### Definitions, Abbreviations and Acronyms

“IL-2/IL-15Rβγ agonist” refers to complex of an IL-2 or IL-2 derivative or an IL-15 or IL-15 derivative targeting the mid-affinity IL-2/IL-15Rβγ and having a decreased or abandoned binding of the IL-2Rα or IL-15Rα. Decreased binding in this context means at least 50%, preferably at least 80% and especially at least 90% decreased binding to the respective Receptor α compared to the wild-type IL-15 or IL-2, respectively. As described and exemplified below, decreased or abandoned binding of IL-15 to the respective IL-15Rα may be mediated by forming a complex (covalent or non-covalent) with an IL-15Rα derivative, by mutations in the IL-15 leading to a decreased or abandoned binding, or by site-specific PEGylation or other post-translational modification of the IL-15 leading to a decreased or abandoned binding. Similarly, decreased or abandoned binding of IL-2 to the respective IL-2Rα may be mediated by mutations in the IL-2 leading to a decreased or abandoned binding, or by site-specific PEGylation or other post-translational modification of the IL-15 leading to a decreased or abandoned binding.

“Interleukin-2”, “IL-2” or “IL2” refers to the human cytokine as described by NCBI Reference Sequence AAB46883.1 or UniProt ID P60568 (SEQ ID NO: 1). Its precursor protein has 153 amino acids, having a 20-aa peptide leader and resulting in a 133-aa mature protein. Its mRNA is described by NCBI GenBank Reference S82692.1.

“IL-2 derivative” refers to a protein having a percentage of identity of at least 92%, preferably of at least 96%, more preferably of at least 98%, and most preferably of at least 99% with the amino acid sequence of the mature human IL-2 (SEQ ID NO: 2). Preferably, an IL-2 derivative has at least about 0.10% of the activity of human IL-2, preferably at least 1%, more preferably at least 10%, more preferably at least 25%, even more preferably at least 50%, and most preferably at least 80%, as determined by a lymphocyte proliferation bioassay. As interleukins are extremely potent molecules, even such low activities as 0.1% of human IL-2 may still be sufficiently potent, especially if dosed higher or if an extended half-life compensates for the loss of activity. Its activity is expresses in International Units as established by the World Health Organization 1^{st} International Standard for Interleukin-2 (human), replaced by the 2^{nd} International Standard (Gearing and Thorpe 1988, Wadhwa et al. 2013). The relationship between potency and protein mass is as follows: 18 million IU PROLEUKIN=1.1 mg protein. As described above, mutations (substitutions) may be introduced in order to specifically link PEG to IL-2 for extending the half-life as done for THOR-707 (Joseph et al. 2019) (WO2019/028419A1) or for modifying the binding properties of the molecule, e.g. reduce the binding to the IL-2a receptor as done for IL2v (Klein et al. 2013, Bacac et al. 2016) (WO 2012/107417A1) by mutation of L72, F42 and/or Y45, especially F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, preferably mutations F42A, Y45A and L72G. Various other mutations of IL-2 have been described: R38W for reducing toxicity (Hu et al. 2003) due to reduction of the vasopermeability activity (US 2003/0124678); N88R for enhancing selectivity for T cells over NK cells (Shanafelt et al. 2000); R38A and F42K for reducing the secretion of proinflammatory cytokines from NK cells ((Heaton et al. 1993) (U.S. Pat. No. 5,229,109); D20T, N88R and Q126D for reducing VLS (US 2007/0036752); R38W and F42K for reducing interaction with CD25 and activation of T_{reg} cells for enhancing efficacy (WO 2008/003473); and additional mutations may be introduced such as T3A for avoiding aggregation and C125A for abolishing O-glycosylation (Klein et al. 2017). Other mutations or combinations of the above may be generated by genetic engineering methods and are well known in the art. Amino acid numbers refer to the mature IL-2 sequence of 133 amino acids.

“Interleukin-15”, “IL-15” or “IL15” refers to the human cytokine as described by NCBI Reference Sequence NP_000576.1 or UniProt ID P40933 (SEQ ID NO: 3). Its precursor protein has 162 amino acids, having a long 48-aa peptide leader and resulting in a 114-aa mature protein (SEQ ID NO: 4). Its mRNA, complete coding sequence is described by NCBI GenBank Reference U14407.1. The IL-15Rα sushi domain (or IL-15Rαₛᵤₛₕᵢ, SEQ ID NO: 6) is the domain of IL-15Rα which is essential for binding to IL-15.

“IL-15 derivative” or “derivative of IL-15” refers to a protein having a percentage of identity of at least 92%, preferably of at least 96%, more preferably of at least 98%, and most preferably of at least 99% with the amino acid sequence of the mature human IL-15 (114 aa) (SEQ ID NO: 4). Preferably, an IL-15 derivative has at least 10% of the activity of IL-15, more preferably at least 25%, even more preferably at least 50%, and most preferably at least 80%. More preferably, the IL-15 derivative has at least 0.1% of the activity of human IL-15, preferably at least 1%, more preferably at least 10%, more preferably at least 25%, even more preferably at least 50%, and most preferably at least 80%. As for IL-2 described above, interleukins are extremely potent molecules, even such low activities as 0.1% of human IL-15 may still be sufficiently potent, especially if dosed higher or if an extended half-life compensates for the loss of activity. Also for IL-15, a plethora of mutations has been described in order to achieve various defined changes to the molecule: D8N, D8A, D61A, N65D, N65A, Q108R for reducing binding to the IL-15Rβγβγ_{c} receptors (WO 2008/143794A1); N72D as an activating mutation (in ALT-803); N1D, N4D, D8N, D30N, D61N, E64Q, N65D, and Q108E to reduce the proliferative activity (US 2018/0118805); L44D, E46K, L47D, V49D, I50D, L66D, L66E, 167D, and 167E for reducing binding to the IL-15Rα (WO 2016/142314A1); N65K and L69R for abrogating the binding of IL-15Rb (WO 2014/207173A1); Q101D and Q108D for inhibiting the function of IL-15 (WO 2006/020849A2); S7Y, S7A, K10A, K11A for reducing IL-15Rβ binding (Ring et al. 2012); L45, S51, L52 substituted by D, E, K or R and E64, 168, L69 and N65 replaced by D, E, R or K for increasing the binding to the IL-15Rα (WO 2005/085282A1); N71 is replaced by S, A or N, N72 by S, A or N, N77 by Q, S, K, A or E and N78 by S, A or G for reducing deamidation (WO 2009/135031A1); WO 2016/060996A2 defines specific regions of IL-15 as being suitable for substitutions (see para. 0020, 0035, 00120 and 00130) and specifically provides guidance how to identify potential substitutions for providing an anchor for a PEG or other modifications (see para. 0021); Q108D with increased affinity for CD122 and impaired recruitment of CD132 for inhibiting IL-2 and IL-15 effector functions and N65K for abrogating CD122 affinity (WO 2017/046200A1); N1D, N4D, D8N, D30N, D61N, E64Q, N65D, and Q108E for gradually reducing the activity of the respective IL-15/IL-15Rα complex regarding activating of NK cells and CD8 T cells (see FIG. 51, WO 2018/071918A1, WO 2018/071919A1). Additionally, or alternatively, the artisan can easily make conservative amino acid substitutions.

The activity of both IL-2 and IL-15 can be determined by induction of proliferation of kit225 cells as described by Hori et al. (1987). Preferably, methods such as colorimetry or fluorescence are used to determine proliferation activation due to IL-2 or IL-15 stimulation, as for example described by Soman et al. using CTLL-2 cells (Soman et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

IL-15 muteins can be generated by standard genetic engineering methods and are well known in the art, e.g. from WO 2005/085282, US 2006/0057680, WO 2008/143794, WO 2009/135031, WO 2014/207173, WO 2016/142314, WO 2016/060996, WO 2017/046200, WO 2018/071918, WO 2018/071919, US 2018/0118805. IL-15 derivatives may further be generated by chemical modification as known in the art, e.g. by PEGylation or other posttranslational modifications (see WO 2017/112528A2, WO 2009/135031).

“IL-2Rα” refers to the human IL-2 receptor a or CD25.

“IL-15Rα” refers to the human IL-15 receptor α or CD215 as described by NCBI Reference Sequence AAI21142.1 or UniProt ID Q13261 (SEQ ID NO: 5). Its precursor protein has 267 amino acids, having a 30-aa peptide leader and resulting in a 231-aa mature protein. Its mRNA is described by NCBI GenBank Reference HQ401283.1. The IL-15Rα sushi domain (or IL-15Rαₛᵤₛₕᵢ, SEQ ID NO: 6) is the domain of IL-15Rα which is essential for binding to IL-15 (Wei et al. 2001). The sushi+ fragment (SEQ ID NO: 7) comprising the sushi domain and part of hinge region, defined as the fourteen amino acids which are located after the sushi domain of this IL-15Rα, in a C-terminal position relative to said sushi domain, i.e., said IL-15Rα hinge region begins at the first amino acid after said (C4) cysteine residue, and ends at the fourteenth amino acid (counting in the standard “from N-terminal to C-terminal” orientation). The sushi+ fragment reconstitutes full binding activity to IL-15 (WO 2007/046006).

“Receptor α” refers to the IL-2Rα or IL-15Rα.

“IL-15Rα derivative” refers to a polypeptide comprising an amino acid sequence having a percentage of identity of at least 92%, preferably of at least 96%, more preferably of at least 98%, and even more preferably of at least 99%, and most preferably 100% identical with the amino acid sequence of the sushi domain of human IL-15Rα (SEQ ID NO: 6) and, preferably of the sushi+ domain of human IL-15Rα (SEQ ID NO: 7). Preferably, the IL-15Rα derivative is a N- and C-terminally truncated polypeptide, whereas the signal peptide (amino acids 1-30 of SEQ ID NO: 5) is deleted and the transmembrane domain and the intracytoplasmic part of IL-15Rα is deleted (amino acids 210 to 267 of SEQ ID NO: 5). Accordingly, preferred IL-15Rα derivatives comprise at least the sushi domain (aa 33-93 but do not extend beyond the extracellular part of the mature IL-15Rα being amino acids 31-209 of SEQ ID NO: 5. Specific preferred IL-15Rα derivatives are the sushi domain of IL-15Rα (SEQ ID NO: 6), the sushi+ domain of IL-15Rα (SEQ ID NO: 7) and a soluble form of IL-15Rα (e.g. from amino acids 31 to either of amino acids 172, 197, 198, 199, 200, 201, 202, 203, 204 or 205 of SEQ ID NO: 5, see WO 2014/066527, (Giron-Michel et al. 2005)) or the extracellular domain of IL-15Rα. Within the limits provided by this definition, the IL-15Rα derivative may include natural occurring or introduced mutations. Natural variants and alternative sequences are e.g. described in the UniProtKB entry Q13261 (https:/www.unprot.org/uniprot/Q13261). Further, the artisan can easily identify less conserved amino acids between mammalian IL-15Rα homologs or even primate IL-15Rα homologs in order to generate derivatives which are still functional. Respective sequences of mammalian IL-15Rα homologs are described in WO 2007/046006, page 18 and 19. Additionally or alternatively, the artisan can easily make conservative amino acid substitutions.

Preferably, an IL-15Rα derivative has at least 10% of the binding activity of the human sushi domain to human IL-15, e.g. as determined in (Wei et al. 2001), more preferably at least 25%, even more preferably at least 50%, and most preferably at least 80%.

“IL-2Rβ” refers to the human IL-Rβ or CD122.

“IL-2Rγ” refers to the common cytokine receptor γ or γ_{c} or CD132, shared by IL-4, IL-7, IL-9, IL-15 and IL-21.

“RLI-15” refers to an IL-15/IL-15Rα complex being a receptor-linker-interleukin fusion protein of the human IL-15Rα sushi+ fragment with the human IL-15. Suitable linkers are described in WO 2007/046006 and WO 2012/175222.

“RLI2” or “SO-C101” are specific versions of RLI-15 and refer to an IL-15/IL-15Rα complex being a receptor-linker-interleukin fusion protein of the human IL-15Rα sushi+ fragment with the human IL-15 (SEQ ID NO: 9) using the linker with the SEQ ID NO: 8.

“ALT-803” (nogapendikin alfa/inbakicept) refers to an IL-15/IL-15Rα complex of Altor BioScience Corp., which is a complex containing 2 molecules of an optimized amino acid-substituted (N72D) human IL-15 “superagonist”, 2 molecules of the human IL-15a receptor “sushi” domain fused to a dimeric human IgG1 Fc that confers stability and prolongs the half-life of the IL-15_{N72D}:IL-15Rαₛᵤₛₕᵢ-Fc complex (see for example US 2017/0088597).

“Heterodimeric IL-15:IL-Ra”, “hetIL-15” or “NIZ985” refer to an IL-15/IL-15Rα complex of Novartis which resembles the IL-15, which circulates as a stable molecular complex with the soluble IL-15Rα, which is a recombinantly co-expressed, non-covalent complex of human IL-15 and the soluble human IL-15Rα (sIL-15Rα), i.e. 170 amino acids of IL-15Rα without the signal peptide and the transmembrane and cytoplasmic domain (see (Thaysen-Andersen et al. 2016, see e.g. table 1) and WO 2021/156720A1 (IL-15 having the SEQ ID NO: 3, the IL-15Rα derivative having the sequences SEQ ID NO: 5 or SEQ ID NO: 14)).

“IL-2/IL-15Rβγ agonists” refers to molecules or complexes which primarily target the mid-affinity IL-2/IL-15Rβγ receptor without binding to the IL-2Rα and/or IL-15Rα receptor, thereby lacking a stimulation of T_{regs}. Examples are IL-15 bound to at least the sushi domain of the IL-15Rα having the advantage of not being dependent on trans-presentation or cell-cell interaction, and of a longer in vivo half-life due to the increased size of the molecule, which have been shown to be significantly more potent that native IL-15 in vitro and in vivo (Robinson and Schluns 2017). Besides IL-15/IL-15Rα based complexes, this can be achieved by mutated or chemically modified IL-2, which have a markedly reduced or timely delayed binding to the IL-2a receptor without affecting the binding to the IL-2/15Rβ and γ_{C} receptor.

“NKTR-214” (bempegaldesleukin) refers to an IL-2/IL-15Rβγ agonist based on IL-2, being a biologic prodrug consisting of IL-2 bound by 6 releasable polyethylene glycol (PEG) chains (WO 2012/065086A1). The presence of multiple PEG chains creates an inactive prodrug, which prevents rapid systemic immune activation upon administration. Use of releasable linkers allows PEG chains to slowly hydrolyze continuously forming active conjugated IL-2 bound by 2-PEGs or 1-PEG. The location of PEG chains at the IL-2/IL-2Rα interface interferes with binding to high-affinity IL-2Rα, while leaving binding to low-affinity IL-2Rβ unperturbed, favoring immune activation over suppression in the tumor (Charych et al. 2016, Charych et al. 2017).

“IL2v” refers to an IL-2/IL-15Rβγ agonist based on IL-2 by Roche, being an IL-2 variant with abolished binding to the IL-2Rα subunit with the SEQ ID NO: 10. IL2v is used for example in fusion proteins, fused to the C-terminus of an antibody. IL2v was designed by disrupting the binding capability to IL-2Rα through amino acid substitutions F42A, Y45A and L72G (conserved between human, mouse and non-human primates) as well as by abolishing O-glycosylation through amino acid substitution T3A and by avoidance of aggregation by a C125A mutation like in aldesleukin (numbering based on UniProt ID P60568 excluding the signal peptide) (Klein et al. 2017). IL2v is used as a fusion partner with antibodies, e.g. with untargeted IgG (IgG-IL2v) in order to increase its half-life (Bacac et al. 2017). In RG7813 (or cergutuzumab amunaleukin, RO-6895882, CEA-IL2v) IL2v is fused to an antibody targeting carcinoembryonic antigen (CEA) with a heterodimeric Fc devoid of FcγR and C1q binding (Klein 2014, Bacac et al. 2016, Klein et al. 2017). And, in RG7461 (or RO6874281 or FAP-IL2v) IL2v is fused to the tumor specific antibody targeting fibroblast activation protein-alpha (FAP) (Klein 2014).

“THOR-707” refers to an IL-2/IL-15Rβγ agonist based on a site-directed, singly PEGylated form of IL-2 with reduced/lacking IL2Rα chain engagement while retaining binding to the intermediate affinity IL-2Rβγ signaling complex (Joseph et al. 2019) (WO 2019/028419A1, P65_30KD molecule).

“ALKS 4230” refers to a circularly permutated (to avoid interaction of the linker with the R and γ receptor chains) IL-2 with the extracellular domain of IL-2Rα selectively targets the βγ receptor as the α-binding side is already occupied by the IL-2Rα fusion component (Lopes et al. 2020).

“P-22339” refers to an IL-15/IL-15Rα sushi complex, where IL-15 is bound to the N-terminus of one Fc chain and the IL-15Rα sushi domain is bound to the N-terminus of a second Fc chain as described in WO 2016/095642 and Hu et al. (2018) with the L52C substitution on the IL-15 polypeptide (SEQ ID NO: 15) and the S40C substitution on the IL-15Rα sushi+ polypeptide (SEQ ID NO: 16) forming a disulfide bond.

“NL-201” refers to IL-2/IL-15Rβγ agonists, which is are computationally designed protein that mimics IL-2 to bind to the IL-2 receptor βγ_{c} heterodimer (IL-2Rβγ) but has no binding site for IL-2Rα or IL-15Rα ((Silva et al. 2019) and WO 2021/081193A1 (NEO 2-15 E62C, SEQ ID NO: 17)).

“NKRT-255” refers to an IL-2/IL-15Rβγ agonist based on a PEG-conjugated human IL-15 that retains binding affinity to the IL-15Rα and exhibits reduced clearance to provide a sustained pharmacodynamic response (WO 2018/213341A1, conjugate 1).

“XmAb24306” refers to an IL-15/IL-15Rα sushi complex, where a mutant IL-15 is bound to the N-terminus of one Fc chain and the IL-15Rα sushi domain is bound to the N-terminus of a second Fc chain as described in as described in US 2018/0118805 (see XENP024306 in FIG. 94C, SEQ ID NO: 18 and SEQ ID NO: 19).

“ANV419” refers to a fusion protein of IL-2 and an IL-2 specific antibody (as described in Huber et al. poster #571, SITC Annual Meeting 2020, Arenas-Ramirez et al. (2016)).

“XTX202” (CLN-617) refers to an engineered IL-2 prodrug with its activity masked as described in WO 2020/069398 and O'Neil J et al. poster ASCO annual meeting 2021.

“AB248” refers to a fusion protein of an anti-CD8 antibody with an IL-2 as described in Moynihan K et al. “Selective activation of CD8+ T cells by a CD8-targeted IL-2 results in enhanced anti-tumor efficacy and safety” poster at SITC 2021.

“WTX-124” refers to a fusion protein of a half-life extension domain, IL-2 and a cleavable inactivation domain as described in Salmeron A. et al., “WTX-124 is an IL-2 Pro-Drug Conditionally Activated in Tumors and Able to Induce Complete Regressions in Mouse Tumor Models”, poster at AACR annual meeting 2021 and WO 2020/232305A1.

“THOR-924, -908, -918” refer to IL-2/IL-15Rβγ agonists based on PEG-conjugated IL-15 with reduced binding to the IL-15Rα with a unnatural amino acid used for site-specific PEGylation (WO 2019/165453A1).

“Percentage of identity” between two amino acids sequences means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by Smith and Waterman (1981), by using the local homology algorithm developed by Needleman and Wunsch (1970), by using the method of similarities developed by Pearson and Lipman (1988), by using computer software using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar 2004), or by using CLUSTAL (Goujon et al. 2010). To get the best local alignment, one can preferably use the BLAST software with the BLOSUM 62 matrix. The identity percentage between two sequences of amino acids is determined by comparing these two sequences optimally aligned, the amino acids sequences being able to encompass additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

“Conservative amino acid substitutions” refers to a substation of an amino acid, where an aliphatic amino acid (i.e. Glycine, Alanine, Valine, Leucine, Isoleucine) is substituted by another aliphatic amino acid, a hydroxyl or sulfur/selenium-containing amino acid (i.e. Serine, Cysteine, Selenocysteine, Threonine, Methionine) is substituted by another hydroxyl or sulfur/selenium-containing amino acid, an aromatic amino acid (i.e. Phenylalanine, Tyrosine, Tryptophan) is substituted by another aromatic amino acid, a basic amino acid (i.e. Histidine, Lysine, Arginine) is substituted by another basic amino acid, or an acidic amino acid or its amide (Aspartate, Glutamate, Asparagine, Glutamine) is replaced by another acidic amino acid or its amide.

“In vivo half-life”, T % or terminal half-life refers to the half-life of elimination or half-life of the terminal phase, i.e. following administration the in vivo half-life is the time required for plasma/blood concentration to decrease by 50% after pseudo-equilibrium of distribution has been reached (Toutain and Bousquet-Melou 2004). The determination of the drug, here the IL-2/IL-15βγ agonist being a polypeptide, in the blood/plasma is typically done through a polypeptide-specific ELISA.

“Immune check point inhibitor”, or in short “check point inhibitors”, refers to a type of drug that blocks certain proteins made by some types of immune system cells, such as T cells, and some cancer cells. These proteins help keeping immune responses in check and can keep T cells from killing cancer cells. When these proteins are blocked, the “brakes” on the immune system are released and T cells are able to kill cancer cells better. Checkpoint inhibitors are accordingly antagonists of immune inhibitory checkpoint molecules or antagonists of agonistic ligands of inhibitory checkpoint molecules. Examples of checkpoint proteins found on T cells or cancer cells include PD-1/PD-L1 and CTLA-4/B7-1/B7-2 (definition of the National Cancer Institute at the National Institute of Health, see htts://www.cancer.ov/publications/dictionaries/cancer-erms/def/immune-checkpointinhibitor) as for example reviewed by Darvin et al. (2018). Examples of such check point inhibitors are anti-PD-L1 antibodies, anti-PD-1 antibodies, anti-CTLA-4 antibodies, but also antibodies against LAG-3 or TIM-3, or blocker of BTLA currently being tested in the clinic (De Sousa Linhares et al. 2018). Further promising check point inhibitors are anti-TIGIT antibodies (Solomon and Garrido-Laguna 2018).

“PD-1 antagonist” or “PD-1 inhibitor” refers to any agent antagonizing or inhibiting the PD-1 checkpoint. PD-1 antagonists or PD-1 inhibitors act to inhibit the association of the programmed death-ligand 1 (PD-L1, CD274) and/or programmed death-ligand 2 (PD-L2, CD273) with its receptor, programmed cell death protein 1 (PD-1, CD279). This interaction is involved in the suppression of the immune system and is used by many cancers to evade the immune system. PD-1 antagonists/inhibitors include anti-PD1 antibodies and anti-PD-L1 antibodies.

“anti-PD-L1 antibody” refers to an antibody, or an antibody fragment thereof, binding to PD-L1. Examples are avelumab, atezolizumab, durvalumab, KN035, MGD013 (bispecific for PD-1 and LAG-3).

“anti-PD-1 antibody” refers to an antibody, or an antibody fragment thereof, binding to PD-1. Examples are pembrolizumab, nivolumab, cemiplimab (REGN2810), BMS-936558, SHR1210, IBI308, PDR001, BGB-A317, BCD-100, JS001.

“anti-PD-L2 antibody” refers to an antibody, or an antibody fragment thereof, binding to anti-PD-L2. An example is sHIgM12.

“an anti-CTLA4 antibody” refers to an antibody, or an antibody fragment thereof, binding to CTLA-4. Examples are ipilimumab and tremelimumab (ticilimumab).

“anti-LAG-3” antibody refers to an antibody, or an antibody fragment thereof, binding to LAG-3. Examples of anti-LAG-3 antibodies are relatlimab (BMS 986016), Sym022, REGN3767, TSR-033, GSK2831781, MGD013 (bispecific for PD-1 and LAG-3), LAG525 (IMP701).

“anti-TIM-3 antibody” refers to an antibody, or an antibody fragment thereof, binding to TIM-3. Examples are TSR-022 and Sym023.

“anti-TIGIT antibody” refers to an antibody, or an antibody fragment thereof, binding to TIGIT. Examples are tiragolumab (MTIG7192A, RG6058) and etigilimab (WO 2018/102536).

“Therapeutic antibody” or “tumor targeting antibody” refers to an antibody, or an antibody fragment thereof, that has a direct therapeutic effect on tumor cells through binding of the antibody to the target expressed on the surface of the treated tumor cell. Such therapeutic activity may be due to receptor binding leading to modified signaling in the cell, antibody-dependent cellular cytotoxicity (ADCC), complement-dependent cytotoxicity (CDC) or other antibody-mediated killing of tumor cells.

“anti-CD38 antibody” refers to an antibody, or an antibody fragment thereof, binding to CD38, also known as cyclic ADP ribose hydrolase. Examples of anti-CD38 antibodies are daratumumab, isatuximab (SAR650984), MOR-202 (MOR03087), TAK-573 or TAK-079 (Abramson 2018) or GEN1029 (HexaBody®-DR5/DR5).

“HPV-induced tumor” or “HPV-induced cancer” refers to a tumor or cancer induced by or associated with a human papilloma virus (HPV) infection. An HPV induced tumor or cancer may be any type of tumor or cancer, including cervical cancer, head-and-neck squamous cell carcinomas, oral neoplasias, oropharyngeal cancer (oropharynx squamous cell carcinoma), penile, anal, vaginal, vulvar cancers and HPV-associated skin cancers (e.g. skin squamous cell carcinoma or keratinocyte carcinoma). An HPV induced tumor or cancer is positive for at least one type of HPV, e.g., by determining presence/expression of the E6 and/or E7 gene/transcript or humoral response to the E6 protein in blood (Augustin et al. 2020, see especially Table 1). The HPV-induced tumor or cancer may be positive for one or more of HPV types 16, 18, 26, 31, 33, 35, 39, 45, 51, 52, 53, 56, 58, 59, 66, 68, 73 and 82, especially types 16, 18, 31, 33 and 45.

When it is stated “administered in combination” this typically does not mean that the two agents are co-formulated and co-administered, but rather one agent has a label that specifies its use in combination with the other. So, for example the IL-2/IL-15Rβγ agonist is for use in treating or managing cancer, wherein the use comprises simultaneously, separately, or sequentially administering the IL-2/IL-15Rβγ agonist and a further therapeutic agent, or vice e versa. But nothing in this application should exclude that the two combined agents are provided as a bundle or kit, or even are co-formulated and administered together where dosing schedules match. So, “administered in combination” includes (i) that the drugs are administered together in a joint infusion, in a joint injection or alike, (ii) that the drugs are administered separately but in parallel according to the given way of administration of each drug, and (iii) that the drugs are administered separately and sequentially.

Parallel administration in this context preferably means that both treatments are initiated together, e.g. the first administration of each drug within the treatment regimen are administered on the same day. Given potential different treatment schedules it is clear that during following days/weeks/months administrations may not always occur on the same day. In general, parallel administration aims for both drugs being present in the body at the same time at the beginning of each treatment cycle.

Sequential administration in this context preferably means that both treatments are started sequentially, e.g. the first administration of the first drug occurs at least one day, preferably a few days or one week, earlier than the first administration of the second drug in order to allow a pharmacodynamic response of the body to the first drug before the second drug becomes active. Treatment schedules may then be overlapping or intermittent, or directly following each other.

The term “resistant to checkpoint inhibitor treatment” refers to a patient that never showed a treatment response when receiving a checkpoint inhibitor.

The term “refractory to checkpoint inhibitor treatment” refers to a patient that initially showed a treatment response to checkpoint inhibitor treatment, but the treatment response was not maintained over time.

The term “about”, when used together with a value, means the value plus/minus 10%, preferably 5% and especially 1% of its value.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the” this includes a plural of that noun unless something else is specifically stated.

The term “at least one” such as in “at least one chemotherapeutic agent” may thus mean that one or more chemotherapeutic agents are meant. The term “combinations thereof” in the same context refers to a combination comprising more than one chemotherapeutic agents.

Technical terms are used by their common sense. If a specific meaning is conveyed to certain terms, definitions of terms will be given in the following in the context of which the terms are used.

“qxw”, from Latin quaque/each, every for every x week, e.g. q2w for every second week, q3w for every third week.

“s.c.” for subcutaneously.

“i.v.” for intravenously.

“i.p.” for intraperitoneally.

### DESCRIPTION OF THE INVENTION

Squamous Cell Carcinoma

In a first aspect, the present invention relates to an interleukin-2/interleukin-15 receptor βγ (IL-2/IL-15Rβγ) agonist for use in the treatment of squamous cell carcinoma in a human patient.

Whereas melanoma and renal cell carcinoma are commonly seen to be indications, where the IL-2/IL-15Rβγ agonists of the invention are expected to show efficacy due to the high immunogenicity of melanoma cells and due to the approval of IL-2 in these indications, the inventors surprisingly observed efficacy in the treatment of squamous cell carcinoma. The inventors observed an about 50%, later even about 60% reduction of the sum of lesions measured by CT scan with contrast agent compared to the CT scan prior to the treatment for a patient with a squamous skin carcinoma, in this case skin squamous cell carcinoma. For a late stage patient who had received, as prior treatments, local radiotherapy, a combination of two chemotherapy modalities (Docetaxel and Cisplatin) together with an anti-cancer antibody (Cetuximab) as a first-line systemic treatment as well as a treatment with an immune check-point inhibitor directed against PD-1 as second line, it was very much surprising that another immuno-oncology drug (i.e. SO-C101) in a single-agent treatment resulted in such a massive reduction of the tumor lesions based on its immuno-oncology mode-of-action alone, as the patient only received SO-C101. After the tumor started again to progress after 4.5 months, the patient was treated with a combination of SO-C101 and another checkpoint inhibitor directed against PD-1 resulting in another 62% tumor reduction within 3 months of treatment. A PET-CT another 1.5 months later showed no “hot spots”, i.e. proliferating tumor. Together with immuno-histochemistry data of different time points of the medical history of the patient it can be concluded that, at the beginning of the SO-C101 treatment, the patient was not responding to the checkpoint inhibitor treatment due to a low level of tumor infiltrating immune effector cells (NK cells, CD8⁺ T cells). Monotherapy with SO-C101 induced a massive activation of immune cells leading to mounting a novel immune response against the tumor, which lead to the initial observed partial response. Despite this treatment success, tumors became resistant to the treatment due to upregulation of PD-L1, silencing the immune effector cells. However, this resistance could be overcome by continuing the treatment with a combination therapy of SO-C101 (i.e. a IL-2/IL-15Rβγ agonist) with an anti-PD-1 antibody (i.e. a checkpoint inhibitor) (see Example 2).

Additionally, further late stage patients showed clinical responses in the combination arm of SO-C101 and pembrolizumab treatment, including a patient with thyroid gland carcinoma (Example 3), a further patient with skin squamous cell carcinoma (Example 4), cervical adenocarcinoma (Example 5) and anus carcinoma (Example 6).

As an interim result, the data show that SO-C101 activates both the innate as well as the adaptive immune response. Surprisingly, in the 6 μg/kg cohort of SO-C101 combined with pembrolizumab, 5 out of 6 patients with late stage tumors (SSCC, cervix uteri, liver, gastric and colorectal) clearly benefited from the treatment (2 patients with partial responses—SSCC and skin melanoma; 3 patients with at least one stable disease—cervix uteri, liver, gastric; all 5 patients still continue treatment), whereas only 1 patient apparently did not profit from the treatment. One patient from this cohort was not counted as the patient discontinued quickly due to an adverse event (colorectal).

Squamous cell carcinoma (SCC) or epidermoid carcinomas is a group of carcinomas that result from degenerated squamous cells forming on the surface of skin and the lining of hollow organs in the body, the respiratory and digestive tracts. A subset of squamous cell carcinomas of the head and neck have been associated with human papilloma virus (HPV) infection (Tumban 2019), such as oral squamous cell carcinoma, oropharyngeal squamous cell carcinoma, and laryngeal squamous cell carcinoma. Further, subsets of anal, penile, vaginal carcinomas are known to be caused by HPV infection. Accordingly, squamous cell carcinomas are preferably selected from the group of skin squamous cell carcinoma (also referred to cutaneous squamous cell carcinoma), non-small-cell lung carcinoma (NSCLC), especially squamous-cell carcinoma of the lung (SCC), squamous cell thyroid carcinoma, head and neck squamous cell carcinoma (HNSCC), oral squamous cell carcinoma, oropharyngeal squamous cell carcinoma, and laryngeal squamous cell carcinoma, esophageal squamous cell carcinoma, esophageal and gastro-esophageal junction cancer squamous cell carcinoma, vaginal squamous-cell carcinoma, penile squamous cell carcinoma, anal squamous cell carcinoma, prostate squamous cell carcinoma, and bladder squamous cell carcinoma. And due to observed association with or even causative role of human papilloma virus (HPV) infection, HPV-associated tumors (Smola 2017, Paradisi et al. 2020) including cervical cancer, head-and-neck squamous cell carcinomas, oral neoplasias, oropharyngeal (notably oropharynx squamous cell carcinoma), penile, anal, vaginal, vulvar cancers and HPV-associated skin cancers (e.g. skin squamous cell carcinoma, keratinocyte carcinoma) (Bouda et al. 2000, Sterling 2005, Howley and Pfister 2015, Augustin et al. 2020) are preferred. Skin squamous cell carcinoma is especially preferred given the treatment success of the patient from Example 2. As the five-year probability of skin squamous cell carcinoma recurrence increases in patients being seropositive for HPV of a high risk type (here HPV-16) (Paradisi et al. 2020), treatment of patients being positive for a high risk type of HPV (types 16, 18, 26, 31, 33, 35, 39, 45, 51, 52, 53, 56, 58, 59, 66, 68, 73 and 82, especially types 16, 18, 31, 33 and 45) is also encompassed by the invention.

HPV detection methods that are currently feasible in the routine practice are HPV PCR, E6/E7 mRNA RT-PCT, E6/E7 mRNA in situ hybridization, HPV DNA in situ hybridization, and P16 immunochemistry. Non-invasive techniques from blood include E6 humoral response and ddPCR-detecting HPVct DNA as well as next-generation sequencing (NGS)-based “capture HPV” is a technique feasible on circulating DNA material (and biopsies) (Augustin et al. 2020, see especially Table 1).

In a preferred embodiment, the patient is (primary) resistant or refractory (due to acquired resistance) to at least one immune checkpoint inhibitor treatment. Checkpoint inhibitors such as PD-1 antagonistic antibodies (e.g. anti-PD-1 antibodies or anti-PD-L1 antibodies) or CTLA-4 antagonistic antibodies (e.g. anti-CTLA-4 antibodies) in the meantime are standard of care for many tumor indications having high response rates. More preferably, the patient is primary resistant or refractory to a PD-1 antagonist, especially to an anti-PD-1 antibody. Still, the majority of patients do not benefit from the treatment (primary resistance), and responders often relapse after a period of response (acquired resistance) (Sharma et al. 2017). Multiple mechanisms may lead or contribute to such resistance toward immunotherapies including absence of antigenic proteins, absence of antigen presentation, genetic T cell exclusion, insensibility of T cells, absence of T cells, (further) inhibitory immune checkpoints or the presence of immunosuppressive cells. Overcoming resistance to immunotherapy is still a huge challenge, and multiple, complex treatment modalities are being tested, including enhancing endogenous T cell function, adoptive transfer of antigen-specific T cells or engineered T cells (CARs or TCRs), vaccinations, molecular targeted strategies, whereas most of the strategies focus on combination strategies and it is concluded that there is an urgent need to test these combination approaches (Sharma et al. 2017). Accordingly, it was not expected that the IL-2/IL-15Rβγ agonists of the invention can lead to the observed treatment success in a patient that was refractory (here likely primary resistance given the observed low infiltration of immune cells prior to the SO-C101 treatment) to an immuno-therapy, in this case to the immune checkpoint inhibitor Cemiplimab, an anti-PD-1 antibody. The effect was observed as a result of a monotherapy with SO-C101, so it must be assumed that the treatment effect resulted only from the activity of the IL-2/IL-15Rβγ agonist.

In one embodiment of the invention, the IL-2/IL-15Rβγ agonist is not administered in combination with an immune checkpoint inhibitor. As observed for the patient of Example 2, no additional treatment was required to achieve a treatment success and the IL-2/IL-15Rβγ agonist surprisingly showed single agent activity. It is therefore one embodiment of the invention to not treat patients with immune checkpoint inhibitors. Cleary, other known or future treatment modalities may still be meaningful to combine with the IL-2/IL-15Rβγ agonist of the invention. Preferably, the patient treated with the IL-2/IL-15Rβγ agonist in absence of an immune checkpoint inhibitor is primary resistant to a PD-1 antagonist, especially to an anti-PD-1 antibody.

In another embodiment of the invention, the IL-2/IL-15Rβγ agonist is not administered in combination with a PD-1 antagonist. As the patient of Example 2 was refractory to a PD-1 antagonist, it is reasonable to assume that patients resistant or refractory to PD-1 antagonist treatment would not further benefit from such treatment if combined with an IL-2/IL-15Rβγ agonist. In one embodiment, the patent is refractory or resistant to PD-1 antagonist treatment.

In a preferred embodiment the IL-2/IL-15Rβγ agonist is not administered in combination with the immune checkpoint inhibitor the patient is refractory or resistant to, preferably wherein the immune checkpoint inhibitor the patient is refractory or resistant to and that not administered in combination is a PD-1 antagonist. As observed for the patient of Example 2, no additional treatment was required to achieve a treatment success and given a resistance to an immune checkpoint inhibitor, it is one embodiment of the invention to not further treat such patient with such immune checkpoint inhibitor. Cleary, other known or future treatment modalities may be meaningful to combine with the IL-2/IL-15Rβγ agonist of the invention.

In one embodiment, the patient had been previously treated with a checkpoint inhibitor. In one embodiment, the patient had been previously treated with a PD-1 antagonist.

In one embodiment, the patient had been previously treated with a checkpoint inhibitor as a monotherapy. In one embodiment, the patient had been previously treated with a PD-1 antagonist as a monotherapy.

In one embodiment, the patient had been previously treated with a checkpoint inhibitor as the sole anti-cancer agent. In one embodiment, the patient had been previously treated with a PD-1 antagonist as the sole anti-cancer agent.

On the other hand, in another embodiment, the IL-2/IL-15Rβγ agonist is administered in combination with an immune checkpoint inhibitor. In another embodiment, the IL-2/IL-15Rβγ agonist is administered in combination with a PD-1 antagonist. Such combinations are meaningful, as the common γ-chain cytokines including IL-2 and IL-15 are known to upregulate the expression of immune checkpoint inhibitors such as PD-1 and its ligands (Kinter et al. 2008). The treatment of a resistant or refractory patient with an IL-2/IL-15Rβγ agonist of the invention may sensitize such patient again for the treatment with an immune checkpoint inhibitor thereby counteracting the resistance mechanism of the tumor. Such effect has been observed for the patient of Example 2, where the patient had been resistant to an anti-PD-1 antibody treatment, responded to SO-C101 treatment with a marked tumor size reduction, however then progressed becoming resistant to SO-C101 treatment, but then responded again to a combined treatment of SO-C101 and pembrolizumab (an anti-PD-1 antibody). It is therefore assumed that SO-C101 lead to a sensitization of the tumor due to upregulation of PD-L1 on tumor cells (which has been observed on tumor biopsies).

Without being bound by any theory, a patient with a low tumor infiltration does not respond/exhibits primary resistance to checkpoint inhibitor treatment, as the tumor has not been recognized by the immune system and therefore the immune response is not yet downregulated through checkpoint inhibitors, e.g. the PD-L1-PD-1 interaction. Treatment with an IL-2/IL-15Rβγ agonist can mount a new immune response which in a second step induces upregulation of the receptor, e.g. PD-1, on immune effector cells, and also may lead for selection of checkpoint, e.g. PD-L1, positive tumor cells, thereby sensitizing the tumor for the checkpoint inhibitor treatment, e.g. a PD-1/PD-L1 targeted checkpoint inhibitor treatment. Also, if a patient was primary resistant or became resistant under treatment to an anti-PD-1 antibody by downregulating PD-1 expression on effector cells, the treatment with an IL-2/IL-15Rβγ agonist would upregulate PD-1 expression again and thereby sensitize the patient (again) to an anti-PD-1 antibody. Further, the IL-2/IL-15Rβγ agonist treatment strongly activated NK cells which de novo can prime an antigen-specific T-cell mediated immune response. Such newly recruited/infiltrating CD8⁺ T cells then would be sensitive to PD-1 blockade again.

In one embodiment of the invention, the IL-2/IL-15Rβγ agonist is the sole anti-cancer agent administered to the patient.

In a preferred embodiment, the IL-2/IL-15Rβγ agonist is administered in combination with an immune checkpoint inhibitor the patient is refractory or resistant to, preferably wherein the immune checkpoint inhibitor the patient is refractory or resistant to and that is administered in combination is a PD-1 antagonist. Based on the potential sensitization of a refractory patient due to the activity of the IL-2/IL-15Rβγ agonist, it would be meaningful to treat a patient even with the immune checkpoint inhibitor, to which the patient was refractory or resistant to. This effect has been observed for the patient of Example 2. Further, the patients from Example 4 and 6 were not responsive/became resistant to anti-PD-1 treatment prior to entering the SO-C101 study in combination with an anti-PD-1 antibody. Given the broad application of PD-1 antagonists as of today and the shown upregulation of PD-1 due to the IL-2/IL-15Rβγ agonist activity, the treatment of PD-1 resistant or refractory patients sensitized by the IL-2/IL-15Rβγ agonists could lead to a huge treatment benefit.

In a preferred embodiment, the treatment of the cancer by the IL-2/IL-15Rβγ agonist of the invention results in at least about 30% size reduction of the tumor present prior to the treatment, preferably about 30% size reduction within 16 weeks of the treatment, preferably about 50% size reduction within 16 weeks of the treatment. For the patient with skin squamous cell carcinoma, a 49% reduction of tumor lesions was observed after 12 weeks of treatment. Tumor size reduction is typically measured by CT scans, with or without contrast agents, magnetic resonance imaging or other imaging techniques, and values obtained prior to the treatment are compared with values at certain time points during or after the treatment (or treatment cycles). One may compare tumor mass/volume or the diameter of tumors. Typically, the value is based on those lesions that were already detectable prior to the treatment (baseline), i.e. new lesions developing during the treatment are not included in such calculation.

In another embodiment the response to the IL-2/IL-15Rβγ agonist is mediated by the innate immune response mediated by NK cells. The highly responsive patient of Example 2, being refractory to an anti-PD-1 antibody potentially due to inactivated/exhausting CD8⁺ T cells, one may speculate that the high number of activated NK cells observed for the patient primed a de novo antigen-specific T-cell mediated immune response, whereas such newly recruited CD8⁺ T cells then would be sensitive to PD-1 blockade again.

In one embodiment, the IL-2/IL-15Rβγ agonist is a complex comprising interleukin 15 (IL-15) or a derivative thereof and interleukin-15 receptor alpha (IL-15Rα) or a derivative thereof. In one embodiment, the complex involves a non-covalent interaction between IL-15 or a derivative thereof and IL-15Rα or a derivative thereof. In one embodiment, the complex involves a covalent bond between IL-15 or a derivative thereof and IL-15Rα or a derivative thereof. The covalent bond may be a disulfide bond between introduced cysteines of a IL-15 derivative and a sushi domain of IL-15Rα derivative (e.g. as described in WO 2016/095642). In one embodiment, the IL-2/IL-15Rβγ agonist is a fusion protein comprising IL-15 or a derivative thereof and IL-15Rα or a derivative thereof. The fusion protein may additionally comprise a flexible linker between IL-15 or a derivative thereof and IL-15Rα or a derivative thereof.

In one embodiment, the derivative of IL-15Rα is a soluble form of IL-15Rα. In one embodiment, the derivative of IL-15Rα is the extracellular domain of IL-15Rα.

In one embodiment, the IL-2/IL-15Rβγ agonist is a complex comprising interleukin 15 (IL-15) or a derivative thereof and the sushi domain of interleukin-15 receptor alpha (IL-15Rα) or a derivative thereof. In one embodiment, the complex involves a non-covalent interaction between IL-15 or a derivative thereof and the sushi domain of IL-15Rα or a derivative thereof. In one embodiment, the complex involves a covalent bond between IL-15 or a derivative thereof and the sushi domain of IL-15Rα or a derivative thereof. The covalent bond may be a disulfide bond between introduced cysteines of a IL-15 derivative and a sushi domain of IL-15Rα derivative (e.g. as described in WO 2016/095642). In one embodiment, the IL-2/IL-15Rβγ agonist is a fusion protein comprising IL-15 or a derivative thereof and the sushi domain of IL-15Rα or a derivative thereof. The fusion protein may additionally comprise a flexible linker between IL-15 or a derivative thereof and the sushi domain of IL-15Rα or a derivative thereof. The flexible linker may comprise SEQ ID NO: 8.

In one embodiment, the sushi domain to IL-15Rα comprises the amino acid sequence of SEQ ID NO: 6 or SEQ ID NO: 7. In one embodiment, IL-15 comprises the amino acid sequence of SEQ ID NO: 4. In one embodiment, the fusion protein comprises the amino acid sequence of SEQ ID NO: 9.

In one embodiment, the IL-2/IL-15Rβγ agonist is selected from the group consisting of
a protein comprising SEQ ID NO: 9,
nogapendikin alfa/inbakicept (ALT-803 as described in US 2017/0088597),
Heterodimeric IL-15:IL-Ra (hetIL-15 or NIZ985) as described in WO 2021/156720A1 (IL-15 having the SEQ ID NO: 3, the IL-15Rα derivative having the sequences SEQ ID NO: 5 or SEQ ID NO: 14), IL-2/IL-15Rβγ agonists as described in Robinson and Schluns (2017),
bempegaldesleukin (NKTR-214 as described in WO 2012/065086A1 and in Charych et al. (2016) and Charych et al. (2017),
IL2v according to SEQ ID NO: 10,
THOR-707 as described in Joseph et al. (2019) and WO 2019/028419A1 (P65_30KD molecule),
Nemvaleukin alfa (ALKS 4230) as described in Lopes et al. (2020)),
P-22339 as described in WO 2016/095642 and Hu et al. (2018) with the L52C substitution on the IL-15 polypeptide (SEQ ID NO: 15) and the S40C substitution on the IL-15Rα sushi+ polypeptide (SEQ ID NO: 16),
NL-201 as described in Silva et al. (2019) and WO 2021/081193A1 (NEO 2-15 E62C, SEQ ID NO: 17),
NKRT-255 as described in WO 2018/213341A1 (conjugate 1),
XmAb24306 as described in US 2018/0118805 (see XENP024306 in FIG. 94C, SEQ ID NO: 18 and SEQ ID NO: 19)
ANV419 fusion protein of IL-2 and an IL-2 specific antibody (as described in Huber et al. poster #571, SITC Annual Meeting 2020, Arenas-Ramirez et al. (2016)),
XTX202 (CLN-617) as described in WO 2020/069398 and O'Neil J et al. poster ASCO annual meeting 2021,
AB248 as described in Moynihan K et al. “Selective activation of CD8⁺ T cells by a CD8-targeted IL-2 results in enhanced anti-tumor efficacy and safety” poster at SITC 2021,
WTX-124 as described in Salmeron A. et al., “WTX-124 is an IL-2 Pro-Drug Conditionally Activated in Tumors and Able to Induce Complete Regressions in Mouse Tumor Models”, poster at AACR annual meeting 2021 and WO 2020/232305A1, and
THOR-924, -908, and -918 as described in WO 2019/165453A1.

In one embodiment, the IL-2/IL-15Rβγ agonist is selected from the group consisting of
(i) a protein comprising the amino acid sequence of SEQ ID NO: 9,
(ii) a protein complex comprising IL-15 comprising the amino acid sequence of SEQ ID NO: 3 and an IL-15Rα derivative comprising the amino acid sequence of SEQ ID NO: 14 or an amino acid sequence corresponding to amino acids 31 to either of amino acids 172, 197, 198, 199, 200, 201, 202, 203, 204 or 205 of SEQ ID NO: 5,
(iii) a protein comprising the amino acid sequence of SEQ ID NO: 10,
(iv) a protein complex comprising IL-15 comprising the amino acid sequence of SEQ ID NO: 15 and an IL-15Rα sushi domain comprising the amino acid sequence of SEQ ID NO: 16,
(v) a protein comprising the amino acid sequence of SEQ ID NO: 17, or
(vi) a protein complex comprising a polypeptide comprising the amino acid sequence of SEQ ID NO: 18 and a polypeptide comprising the amino acid sequence of SEQ ID NO: 19.

Pulsed Cyclic Dosing

In another aspect, the present invention relates to an IL-2/IL-15Rβγ agonist according to the present invention, comprising administering the IL-2/IL-15Rβγ agonist to a human patient using a cyclical administration regimen, wherein the cyclical administration regimen comprises:
(a) first period of x days during which the IL-2/IL-15Rβγ agonist is administered at a daily dose on γ consecutive days at the beginning of the first period followed by x-y days without administration of the IL-2/IL-15Rβγ agonist, wherein x is 5, 6, 7, 8, 9, 10, 11, 12, 13, 14, 15, 16, 17, 18, 19, 20 or 21 days, preferably, 7 or 14 days, and y is 2, 3 or 4 days, preferably 2 or 3 days;
(b) repeating the first period at least once; and
(c) a second period of z days without administration of the IL-2/IL-15Rβγ agonist, wherein z is 5, 6, 7, 8, 9, 10, 11, 12, 13, 14, 15, 16, 17, 18, 19, 20, 21, 28, 35, 42, 49, 56, 63 or 70 days, preferably 7, 14, 21 or 56 days, more preferably 7, 14 or 21 days. For illustration, a graphical representation of the dosing is depicted in FIG. 6. In a more preferred embodiment, y is 2 days and x is 7 days.

In a another aspect, the present invention relates to an interleukin-2/interleukin-15 receptor βγ (IL-2/IL-15Rβγ) agonist for use in treating or managing cancer, comprising administering the IL-2/IL-15Rβγ agonist to a human patient using a cyclical administration regimen, wherein the cyclical administration regimen comprises:
(a) a first period of x days during which the IL-2/IL-15Rβγ agonist is administered at a daily dose on y consecutive days at the beginning of the first period followed by x-y days without administration of the IL-2/IL-15Rβγ agonist, wherein x is 5, 6, 7, 8 or 9 days, and y is 2, 3 or 4 days;
(b) repeating the first period at least once; and
(c) a second period of z days without administration of the IL-2/IL-15Rβγ agonist, wherein z is 5, 6, 7, 8, 9, 10, 11, 12, 13, 14, 15, 16, 17, 18, 19 or 20 days. For illustration, a graphical representation of the dosing is depicted in FIG. 6.

This administration scheme can be described as a “pulsed cyclic” dosing—“pulsed” as the IL-2/IL-15Rβγ agonist is administered e.g. at day 1 and day 2 of a week activating and expanding both NK and CD8⁺ T cells (a “pulse”), followed by no administration of the agonist for the rest of the week (step (a). This on/off administration is repeated at least once, e.g. for two or three weeks (step (b)), followed by another period without an administration of the IL-2/IL-15Rβγ agonist, e.g. another week (step (c)). Accordingly, examples of a cycle are (a)-(a)-(c) ((a) repeated once) or (a)-(a)-(a)-(c) ((a) repeated twice). Pulsed dosing occurs in the first period according to step (a) and in the repetition of the first period in step (b). Step (a), (b) and (c) together, i.e., the pulsed dosing in combination with the second period without administration of the IL-2/IL-15Rβγ agonist, are referred to as one cycle or one treatment cycle. This whole treatment cycle (first periods and second period) may then be repeated multiple times.

The inventors surprisingly found that in cynomolgus monkeys the pulsed dosing of the IL-2/IL-15Rβγ agonist RLI-15/SO-C101 on consecutive days lead to a strong, dose dependent activation of NK cells and CD8⁺ T cells (measured by determining the expression of Ki67, i.e. becoming Ki67⁺) both for i.v. and s.c. administration. At the same time T_{regs} were not induced. It was surprising that after a 1^{st} administration of an IL-2/IL-15Rβγ agonist in primates on day 1, a 2^{nd} administration of the same dose on day 2 lead to a further increase in activation of both NK cells and CD8⁺ T cells. A 4^{th} administration on day 4 did not result in a further increase of activation, but still kept the activation levels high. A rest period of several days was then sufficient to achieve similar levels of activation in a second pulse.

RLI-15 provides optimal activation of NK cells and CD8⁺ T cells with two consecutive daily doses per week in primates. This is surprising given the relatively short half-life of RLI-15, leading to high levels of proliferating NK cells and CD8⁺ T cells still 4 days after the first, and 3 days after the second dosing.

A long-term continuous stimulation of the mid-affinity IL-2/IL-15Rβγ receptor may not provide any additional benefit in the stimulation of NK cells and CD8⁺ T cells compared to relative short stimulation by two consecutive daily doses with a relative short-lived IL-2/IL-15Rβγ receptor agonist such as RLI-15. To the contrary, continuous stimulation by too frequent dosing or agonists with significantly longer half-life may even cause exhaustion and anergy of the NK cells and CD8⁺ T cells in primates.

The pulsed cyclic dosing provided herein is in contrast to previously described dosing regimens for IL-2/IL-15Rβγ agonist tested in primates and humans applying continuous dosing of such agonists, trying to optimize AUC and C, over time similar to a classical drug, i.e. aiming for constant drug levels and hence continuous stimulation of the effector cells.

For example, IL-2 and IL-15 are dosed continuously: IL-2 i.v. bolus over 15 min every 8 hours; and IL-15 s.c. days 1-8 and 22-29, or i.v. continuous infusion for 5 or 10 consecutive days, or i.v. daily for 12 consecutive days (see clinical trials: NCT03388632, NCT01572493, NCT01021059). The IL-2/IL-15Rβγ agonist hetIL-15 was dosed in primates continuously on days 1, 3, 5, 8, 10, 12 and 29, 31, 33, 36, 38 and 40 (i.e. always day 1, 3 and 5 of a week). A lack of responsiveness was tried to be overcome by increasing the dose of the IL-2/IL-15Rβγ agonist up to rather high doses of 64 μg/kg (Bergamaschi et al. 2018), much higher than tolerated in humans (Conlon et al. 2019). In humans hetIL-15 (NIZ985) was dosed at 0.25 to 4.0 μg/kg 2 weeks-on/2 weeks-off administered s.c. again three times a week (TIW) (Conlon et al. 2019). In comparison, the ALT-803 was administered in a human clinical trial once per week (on weeks 1 to 5 of four 6-week cycles) (Wrangle et al. 2018). And NKT-214 is dosed once every 3 weeks.

The finding of the inventors was further in contrast to report by Frutoso et al., where in a pulsed dosing in mice (day 1 and day 3 followed by a treatment break) the second stimulation with IL-15 or an IL-2/IL-15Rβγ agonist did not lead to a marked activation of NK cells in vivo (Frutoso et al. 2018).

In one embodiment the IL-2/IL-15Rβγ agonist is for use in the cyclic administration regimen, wherein x is 6, 7 or 8 days, preferably 7 days. For convenience reasons, it is advantageous that patients are treated in weekly rhythm, especially if such rhythm is to be repeated over many weeks, i.e. x is preferably 7 days, but one can reasonably assume that changing the rhythm to 6 or 8 days would not have a major impact on the treatment result making 6 or 8 days also preferred embodiments.

In another embodiment, the IL-2/IL-15Rβγ agonist is for use in the cyclic administration regimen, wherein y is 2 or 3 days, preferably 2 days. It was shown in the cynomolgus monkeys that optimal activation (measures as Ki67⁺) of both NK cells and CD8⁺ T cells can be reached by 2 daily administrations per week on 2 consecutive days, whereas 4 daily consecutive administrations within one week did not provide any additional benefit with respect to activated NK cells and CD8⁺ T cells. In other words, the activation of NK cells and CD8⁺ T cells reached a plateau between the 2^{nd} and the 4^{th} administration. Accordingly, 2 and 3, more preferably 2 consecutive daily administrations are preferred in order to minimize exposure of the patient to the drug, but still achieve high levels of activation of the effector cells.

In another embodiment the IL-2/IL-15Rβγ agonist is for use in the cyclic administration regimen, wherein z is 6, 7 or 8 days. In order to stay in a weekly rhythm for convenience of the patients, the period z, where no administration of the IL-2/IL-15Rβγ agonist occurs, is preferably 7 or 14 days, more preferably 7 days.

The dosing regimen according to the invention may be preceded by a pre-treatment period, where the IL-2/IL-15Rβγ agonist is dosed at a lower daily dose, administered less frequently or where an extended treatment break is applied in order to test the response of the patient or get the patient used to the treatment or prime the immune system for a subsequent higher immune cell response. For example it is envisaged that there is one additional treatment cycle as pre-treatment with y days of treatment (e.g. 2 or 3 days) in the treatment period x (e.g. 7 days), whereas z is extended compared to the following treatment cycles (e.g. 14 days instead of 7 days).

In an especially preferred embodiment the IL-2/IL-15Rβγ agonist is for use in the cyclic administration regimen, wherein x is 7 days, y is 2 days and z is 7 days. This especially preferred treatment cycle of 2 administrations on 2 consecutive days, followed by 7−2=5 days without administration and therefore making a weekly cycle combines the minimal exposure of 2 administrations of the IL-2/IL-15Rβγ agonist achieving the maximum activation of the NK cells and CD8⁺ T cells with the convenient weekly cycling for the patient. The first-in-human clinical trial with RLI-15/SO-C101 as monotherapy is presently conducted according to this scheme with treatment at day 1 and day 2, followed by 5 days of non-treatment to complete the first week/period (i.e. x=7; y is 2), this first treatment period is repeated once and followed by one week with no administration (z=7). This 21 day cycle is then repeated until disease progression.

In an especially preferred embodiment the IL-2/IL-15Rβγ agonist is for use in the cyclic administration regimen, wherein x is 7 days, y is 2, 3 or 4 days and z is 7 days. Whereas 2 administrations on 2 consecutive days already showed already maximum activation of NK cells and CD8+ cells, 4 administrations on 4 consecutive days maintained such activation for another two days without leading to a marked decrease of activated NK cells and CD8+ cells. Therefore, an alternative preferred treatment regimen is, wherein x is 7 days, y is 3 days and z is 7 days, i.e. 3 administrations on 3 consecutive days followed by 7−3=4 days without administration, which may be beneficial if a prolonged activation of the NK cells and CD8⁺ T cells translates into higher efficacy. And, another alternative preferred treatment regimen is, wherein x is 7 days, y is 4 days and z is 7 days, i.e. 4 administrations on 4 consecutive days followed by 7−4=3 days without administration, which may be beneficial if a prolonged activation of the NK cells and CD8⁺ T cells translates into higher efficacy.

In one embodiment, the IL-2/IL-15Rβγ agonist is for use in the cyclic administration regimen, wherein the daily dose is 0.1 μg/kg (0.0043 uM) to 50 μg/kg (2.15 uM) of the IL-2/IL-15Rβγ agonist.

In one embodiment the IL-2/IL-15Rβγ agonist is for use in the cyclic administration regimen, wherein the daily dose is 0.0043 μM to 2.15 μM of the IL-2/IL-15Rβγ agonist.

The present inventors could show a good correlation between RLI-15/SO-C101 (for which 1 μM equals 23 μg/kg) and NK and CD8⁺ T cell proliferation in vitro for human NK cells and CD8⁺ T cells and in vivo data obtained from cynomolgus monkeys. From this correlation, it is possible to predict the Minimal Anticipated Biologic Effect Level (MABEL) at about 0.25 μg/kg, the Pharmacologic Active Doses (PAD) at between about 0.6 μg/kg and 10 μg/kg together with the No Observed Adverse Effect Level (NOAEL) at about 25 μg/kg and the Maximum Tolerated Dose (MTD) at about 32 μg/kg for RLI-15 and IL-2/IL-15Rβγ agonists, preferably of an IL-2/IL-15Rβγ agonist with about the same molecular weight. These values equal a MABEL of about 0.011 μM of the IL-2/IL-15Rβγ agonist, a PAD at between about 0.026 μM and 0.43 μM of the IL-2/IL-15Rβγ agonist, a NOAEL at about 1.1 μM of the IL-2/IL-15Rβγ agonist and the MTD at about 1.38 μM of the IL-2/IL-15Rβγ agonist.

Considering potential deviations from the predictions, a starting dose of 0.1 μg/kg (0.0043 μM) for a clinical trial has been determined and the observed MTD in humans may be up to 50 μg/kg (2.15 μM). Preferably, the dose is between 0.25 μg/kg (0.011 μM) (MABEL) and 25 μg/kg (1.1 μM) (NOAEL), more preferably between 0.6 μg/kg (0.026 μM) and 10 μg/kg (0.43 μM) (PAD), more preferably from 1 μg/kg (0.043 μM) to 15 μg/kg (0.645 μM), and especially 2 μg/kg (0.087 μM) to 12 μg/kg (0.52 μM).

Accordingly, in another embodiment, the IL-2/IL-15Rβγ agonist is for use in the cyclic administration regimen, wherein the daily dose is 0.0043 μM to 2.15 μM of the IL-2/IL-15Rβγ agonist, preferably the dose is between 0.011 μM (MABEL) and 1.1 μM (NOAEL), and more preferably between 0.026 μM and 0.52 μM (PAD).

In a preferred embodiment the IL-2/IL-15Rβγ agonist is for use in the cyclic administration regimen, wherein the daily dose selected within the dose range of 0.1 to 50 μg/kg, preferably 0.25 to 25 μg/kg, more preferably 0.6 to 12 μg/kg and especially 2 to 12 μg/kg, is not substantially increased during the administration regimen, preferably wherein the dose is maintained during the administration regime. Surprisingly, the administration regimen according to the invention showed repeated activation of NK cells and CD8⁺ T cells and did not require a dose increase over time. This has not been observed for example in the dose regimen used for hetIL-15, which was compensated by progressively doubling doses from 2 to 64 μg/kg (Bergamaschi et al. 2018). Therefore, it is an important advantage that the selected daily dose within the range of 0.1 to 50 μg/kg does not have to be increased within repeating the first period of administration, or from one cycle to the next. This enables repeated cycles of the treatment without running the risk of getting into toxic doses or that the treatment over time becomes ineffective. Further, maintaining the same daily dose during the administration regimen ensures higher compliance as doctors or nurses do not need to adjust the doses from one treatment to another.

In one embodiment, the IL-2/IL-15Rβγ agonist is for use in the cyclic administration regimen, wherein the daily dose is 3 μg/kg (0.13 μM) to 20 μg/kg (0.87 μM), preferably 6 μg/kg (0.26 μM) to 12 μg/kg (0.52 μM) of the IL-2/IL-15Rβγ agonist.

In one embodiment the IL-2/IL-15Rβγ agonist is for use in the cyclic administration regimen, wherein the daily dose is a fixed dose independent of body weight of 7 μg to 3500 μg (0.30 mol to 150 mol), preferably 17.5 μg to 1750 μg (0.76 mol to 76 mol), more preferably 42 μg to 700 μg (1.8 mol to 30 mol) and especially 140 μg to 700 μg (6.1 mol to 30 mol).

In one embodiment the IL-2/IL-15Rβγ agonist is for use in the cyclic administration regimen, wherein the daily dose is increased during the administration regime. As the IL-2/IL-15Rβγ agonist leads to an expansion of the cells expressing the IL-2/IL-15Rβγ receptor and to an enhanced expression of the receptor on the surface, equal doses of the agonist will over time lead to a decreased plasma concentration of the agonist, as more agonist molecules will be bound to the cells. In order to compensate for the molecules being more and more captured by the target cells, the daily dose is preferably increased during the administration regime.

Such increase of the daily dose may preferably occur after each period of x days. Typically, such increases can best operationally be managed if increases occur after each pulse of x days. Especially CD8⁺ T cells appear to lose sensitivity to stimulation by the IL-2/IL-15Rβγ agonist after a pulse treatment of x days. Accordingly, it is preferred the increase the daily dose after each pulse of x days (until the upper limit of a tolerated daily dose is reached).

In one embodiment, the next treatment cycle starts again at the initial daily dose and is increased again after each pulse of x days (see FIG. 6, option A). Alternatively, the next treatment cycle starts with the same daily dose as the last daily (increased) dose of the previous pulse of x days) (see FIG. 6, option B).

In one embodiment, the daily dose is increased by about 20% to about 100%, preferably by about 30% to about 50% after each period of x days in order to compensate for the expansion of the target cells.

Such increases would be limited by an upper limit, which cannot be exceeded due to e.g. dose limiting toxicities. Given the binding of the agonist to the target cells, this upper limit is however expected to dependent on the number of target cells, i.e. a patient with an expanded target cell compartment is expected to tolerate a higher dose of the agonist compared to an (untreated) patient with a lower number of target cells. Still, it is assumed that upper limit of a tolerated daily dose after dose increases is 50 μg/kg (2.15 μM), preferably 32 μg/kg (1.4 μM), more preferably 20 μg/kg (0.87 μM) and especially 12 μg/kg (0.52 μM).

In another embodiment, the daily dose is increased only once after the first period of x days, preferably by about 20% to about 100%, preferably by about 30% to about 50% after the first period of x days. Already one increase of the daily dose may reach the upper limit of a tolerated daily dose and further, during the z days without administration of the IL-2/IL-15Rβγ agonist levels of NK cells and CD8⁺ cells are expected to go back to nearly normal levels making one increase sufficient.

In another embodiment, the daily dose is increased after each daily dose within the pulse period y. Preferred embodiments are that for the next treatment period x within the same cycle, the next daily dose may then be further increased (see FIG. 6, option C) or continue at the same daily dose level as the last daily dose of the previous treatment period x (see FIG. 6, option D). Between treatment cycles, the daily dose may always start again at the initial dose level (see FIG. 6, option C and B) or continue at the increased dose level from the first treatment day of the preceding treatment period x (see FIG. 6, option E). Again, such increases would be limited by an upper limit, which cannot be exceeded due to e.g. dose limiting toxicities. Given the binding of the agonist to the target cells, this upper limit is however expected to dependent on the number of target cells, i.e. a patient with an expanded target cell compartment is expected to tolerate a higher dose of the agonist compared to an (untreated) patient with a lower number of target cells. Still, it is assumed that upper limit of a tolerated daily dose after dose increases is 50 μg/kg (2.15 μM), preferably 32 μg/kg (1.4 μM) and especially 20 μg/kg (0.87 μM).

In one embodiment the IL-2/IL-15Rβγ agonist is for use wherein the daily dose is administered in a single injection. Single daily injections are convenient for patients and healthcare providers and are therefore preferred.

However, given the short half-life of the molecule and the hypothesis that the activation of the immune cells being dependent on the increase of IL-2/IL-15Rβγ agonists rather than on continuous levels of such agonist, it is another preferred embodiment that the daily dose is split into 2 or 3 individual doses that are administered within one day, wherein the time interval between administration of the individual doses is at least about 4 h and preferably not more than 12 h (dense pulsed cyclic dosing). It is expected that the same amount of the agonist—split into several doses and administered during the day—is more efficacious in stimulating in human patients NK cells and especially CD 8⁺ cells, the latter showing a lower sensitivity for the stimulation, than administered only in a single injection. This has surprisingly been observed in mice. Practically, such multiple dosing should be able to be integrated into the daily business of hospitals, doctor's practice or outpatient settings and therefore, 2 to 3 equal doses administered during business hours including shifts between 8 and 12 hours would still be conveniently manageable, with 8 or 10 h intervals being preferred as the maximum time difference between first and last dose. Accordingly, it is a preferred embodiment that the daily dose is split into 3 individual doses that are administered within one day, wherein the time interval between administration of the individual doses is about 5 to about 7 h, preferably about 6 hours. This means that a patient could be dosed e.g. at 7 am, 2 pm and 7 pm every day (with 6-hour intervals), or at 7 am, 1 pm and 6 pm (with 5-hour intervals). In another preferred embodiment, the daily dose is split into 2 individual doses that are administered within one day, wherein the time interval between administration of the individual doses is about 6 h to about 10 h, preferably 8 h. In the case of 2 doses, a patient could be dosed e.g. at 8 am and 4 pm (with an 8-hour interval). Given the daily routine of hospitals, the intervals between the administrations may vary within a day or from day to day.

In another preferred embodiment, the IL-2/IL-15Rβγ agonist is for use in the cyclic administration regimen, wherein the IL-2/IL-15Rβγ agonist is administered subcutaneously (s.c.) or intraperitoneally (i.p.), preferably s.c. The inventors observed in a cynomolgus study that s.c. administration was more potent than i.v. administration with regards to activation of NK cells and CD8⁺ T cells. ip. administration has similar pharmacodynamics effects as s.c. administration. Therefore, i.p. administration is another preferred embodiment, especially for cancers originating from organs in the peritoneal cavity, e.g. ovarian, pancreatic, colorectal, gastric and liver cancer as well as peritoneal metastasis owing to locoregional spread and distant metastasis of extraperitoneal cancers.

In another embodiment, the IL-2/IL-15Rβγ agonist is for use in the cyclic administration regimen, wherein administration of the IL-2/IL-15Rβγ agonist in step (a) results in an increase of the % of Ki-67⁺ NK of total NK cells in comparison to no administration of the IL-2/IL-15Rβγ agonist, and wherein administration of the IL-2/IL-15Rβγ agonist in step (b) results in a Ki-67⁺ NK cell level that is at least 70% of the of the Ki-67⁺ NK cells of step (a). Ki-67 is a marker for proliferating cells and therefore percentage of Ki-67⁺ NK cell of total NK cells is a measure to determine the activation state of the respective NK cell population. It was surprisingly shown that repeating daily consecutive administrations after x-y days without administration of the agonist lead again to a strong activation of NK cells, which was at least 70% of the level of activation of the NK cells during the first period with daily administrations for x days (step a). The level of NK cell activation is measured as % of Ki-67+NK cells of total NK cells.

Still, in another embodiment the IL-2/IL-15Rβγ agonist is for use in the cyclic administration regimen, wherein the IL-2/IL-15Rβγ agonist administration results in maintenance of NK cell numbers or preferably an increase of NK cell numbers to at least 110% as compared to no administration of IL-2/IL-15Rβγ agonist after at least one repetition of the first period, preferably after at least two repetitions of the first period. Alternatively or additionally to measuring the NK cell activation, also total numbers of NK cells matter and it was shown that repeating daily consecutive administrations after x-y days without administration of the agonist lead on average to an increase in total numbers of NK cells over one or two repetitions of the first period (a). In absolute numbers the IL-2/IL-15Rβγ agonist administration resulted in NK cell numbers of at least about 1.1×10³ NK cells/μl after at least one repetition of the first period, preferably after at least two repetitions of the first period.

In another embodiment the IL-2/IL-15Rβγ agonist is for use in the cyclic administration regimen, wherein the cyclic administration of is repeated over at least 3 cycles, preferably 5 cycles, more preferably at least 10 cycles and even more preferably until disease progression. Given the inventors' finding that, after an initial strong activation of NK cells and CD8⁺ T cells in the phase 1 of the pharmacokinetic and pharmacodynamics study in the cynomolgus monkey by 4 consecutive daily administrations, followed by a treatment break of 18 days, NK cells and CD8⁺ T cells can again be strongly activated, it can be reasonably concluded that the 2 or 3 repetitions of the daily administrations on consecutive days can be again repeated after a treatment break. Accordingly, repetition of at least 3 cycles, preferably 5 cycles or preferably at least 10 cycles for boosting the immune system are foreseen. As tumors often develop resistance to most treatment modalities, for the treatment of tumors it is especially foreseen to repeat cycles until disease progression.

In another embodiment, the IL-2/IL-15Rβγ agonist is for use in the cyclic administration regimen, wherein the IL-2/IL-15Rβγ agonist has an in vivo half-life of 30 min to 24 h, preferably 1 h to 12 h, more preferably of 2 h to 6 h. Preferably, the in vivo half-life is the in vivo half-life as determined in mouse of 30 min to 12 h, more preferably 1 h to 6 h. In another preferred embodiment, the in vivo half-life is the in vivo half-life as determined in cynomolgus or macaques of 1 h to 24 h, more preferably of 2 h to 12 h. In another embodiment the in vivo half-life as determined in cynomolgus monkeys is 30 min to 12 hours, more preferably 30 min to 6 hours.

Pharmacokinetic and pharmacodynamic properties of the IL-2/IL-15Rβγ agonists of the invention depend on the in vivo half-life of such agonists. Due to various engineering techniques the in vivo half-life has been increased, e.g. by creating larger proteins by fusion to an Fc part of an antibody (e.g. ALT-803, R0687428) or antibodies (RG7813, RG7461, immunocytokines of WO 2012/175222A1, WO 2015/018528A1, WO 2015/109124) or PEGylation (NKT-214). However, a too long half-life may actually stimulate NK cells for too long, leading to a preferential accrual of mature NK cells with altered activation and diminished functional capacity (Elpek et al. 2010, Felices et al. 2018). Therefore, the preferred IL-2/IL-15Rβγ agonist has an in vivo half-life of 30 min to 24 h, preferably 1 h to 12 h, more preferably of 2 h to 6 h, or preferably 30 min to 12 hours, more preferably 30 min to 6 hours. Preferably, this in vivo half-life refers to the half-life in humans. However, as the determination of the in vivo half-life in humans, if not published, may be unethical to determine, it is also preferred to use the in vivo half-life of mice or primates such as cynomolgus monkeys or macaques. Given the generally shorter half-life in mice, the in vivo half-life as determined in mouse is preferably. 30 min to 12 h, more preferably 1 h to 6 h or 30 min to 6 h, and the in vivo half-life as determined in cynomolgus or macaques of 1 h to 24 h, more preferably of 2 h to 12 h or 30 min to 6 h.

In another embodiment, the IL-2/IL-15Rβγ agonist is for use in the cyclic administration regimen, wherein the IL-2/IL-15Rβγ agonist is at least 70% monomeric, preferably at least 80% monomeric. Aggregates of such agonists may also have an impact on the pharmacokinetic and pharmacodynamic properties of the agonists and therefore should be avoided in the interest of reproducible results.

In another preferred embodiment, the IL-2/IL-15Rβγ agonist is for use in the cyclic administration regimen, wherein the IL-2/IL-15Rβγ agonist is an interleukin 15 (IL-15)/interleukin-15 receptor alpha (IL-15Rα) complex. IL-15/IL-15Rα complexes, i.e. complexes (covalent or non-covalent) comprising an IL-15 or derivative thereof and at least the sushi domain of the IL-15Rα or derivative thereof. They target the mid-affinity IL-2/IL-15Rβγ, i.e. the receptor consisting of the IL-2/IL-15Rβ and the γ_{c} subunits, which is expressed on NK cells, CD8⁺ T cells, NKT cells and γδ T cells. These complexes are well-known in the art and their binding capabilities are well understood, whereas other attempts by modifying IL-2 to reduce/abandon IL-2Rα binding or synthetic approaches may face unpredictable risks. Preferably, the complex comprises a human IL-15 or a derivative thereof and the sushi domain of IL-15Rα (SEQ ID NO: 6), the sushi+ domain of IL-15Rα (SEQ ID NO: 7) or a soluble form of IL-15Rα (from amino acids 31 to either of amino acids 172, 197, 198, 199, 200, 201, 202, 203, 204 or 205 of SEQ ID NO: 5, see WO 2014/066527, (Giron-Michel et al. 2005)).

In a more preferred embodiment, the IL-15/IL-15Rα complex is a fusion protein comprising the human IL-15Rα sushi domain or derivative thereof, a flexible linker and the human IL-15 or derivative thereof, preferably wherein the human IL-15Rα sushi domain comprises the sequence of SEQ ID NO: 6, more preferably comprising the sushi+ fragment (SEQ ID NO: 7), and wherein the human IL-15 comprises the sequence of SEQ ID NO: 4. Such fusion protein is preferably in the order (from N- to C-terminus) IL-15_Ra-linker-IL-15 (RLI-15). An especially preferred IL-2/IL-15Rβγ agonist is the fusion protein designated RLT2 (SO-C101) having the sequence of SEQ ID NO: 9.

In an especially preferred embodiment, the IL-15/IL-15Rα is the molecule registered under CAS Registry Number 1416390-27-6.

In another embodiment, the IL-2/IL-15Rβγ agonist is for use in the cyclic administration regimen, wherein a further therapeutic agent is administered in combination with the IL-2/IL-15Rβγ agonist. In the past years, cancer therapies are typically combined with existing or new therapeutic agents in order to tackle tumors through multiple mode of actions. At the same time, it is difficult or unethical to replace established therapies by new therapies, so typically new therapies are combined with the standard of care in order to achieve an additional benefit for the patient. Accordingly, also for the provided dosing regimens, these have to be combined with regimens of other therapeutic drugs. The further therapeutic agent and the IL-2/IL-15Rβγ agonist may be administered on the same days and/or on different days. Administration on the same day typically is more convenient for the patients as it minimizes visits to the hospital or doctor. On the other hand, scheduling the administration for different days may become important for certain combinations, where there may be an unwanted interaction between the agonist of the invention and another drug.

As the typical clinical development path is the combination with standard of care, the administration of the combination agent is maintained and therefore is independent of the administration regimen of the IL-2/IL-15Rβγ agonist.

In another embodiment, the IL-2/IL-15Rβγ agonist is for use in the cyclic administration regimen, wherein the further therapeutic agent is an immune checkpoint inhibitor (or in short checkpoint inhibitor) or a therapeutic antibody.

Preferably, the checkpoint inhibitor or the therapeutic antibody is administered at the beginning of each period (a) of each cycle. In order to warrant high compliance with the timely dosing of the therapeutic agents and to minimize procedures, the treatment cycles of the agonist and the checkpoint inhibitor or the therapeutic antibody are ideally started together, e.g. in the same week. Depending on potential interactions between the agonist and the combined antibody, this may be the same day, or at different days in the same week. For example, expanding the NK cells and CD8⁺ T cells first for 1, 2, 3 or 4 days before adding the checkpoint inhibitor or the therapeutic antibody may result in improved efficacy of the treatment.

In one embodiment, the IL-2/IL-15Rβγ agonist is for use, wherein the x days and z days are adapted that an integral multiple of x days+z days (n×x+z with n c {2, 3, 4, 5, . . . }) equal the days of one treatment cycle of the checkpoint inhibitor or the therapeutic antibody, or, if the treatment cycle of the checkpoint inhibitor or the therapeutic antibody changes over time, equal to each individual treatment cycle of the checkpoint inhibitor or the therapeutic antibody.

For example, checkpoint inhibitors or therapeutic antibody are typically dosed every 3 or every 4 weeks. For example, the treatment schedule of the IL-2/IL-15Rβγ agonist of the present inventions matches with the treatment schedule of a checkpoint inhibitor, if both the IL-2/IL-15Rβγ agonist and the checkpoint inhibitor are administered at the beginning of the first period (a) (treatment period x), preferably at the first day of the first period (a), and the checkpoint inhibitor or therapeutic antibody is not further administered for the rest of the treatment cycle. For every following treatment cycle the check point inhibitor or therapeutic antibody is then again administered at the beginning, preferably on the first day, of period (a). Accordingly, if x is 7 (i.e. a week) and (a) is repeated once (so the integral multiple n is 2) and z is 7, the checkpoint inhibitor or therapeutic antibody would be administered every 3 weeks (2×7+7=3 weeks), or, if x is 7 and (a) is repeated twice (so the integral multiple n is 3) and z is 7, the checkpoint inhibitor or therapeutic antibody would be administered every 4 weeks (3×7+7=4 weeks). In case of a 6-week schedule of the checkpoint inhibitor or therapeutic antibody, the agonist may either be scheduled as to 3 week cycles (2×7+7) or one 6 week cycle (5×7+7 or 4×7+14). In case the treatment regimen of the checkpoint inhibitor or therapeutic antibody is changed overtime, typically, the rhythm of the schedules is adapted by extending the period z to synchronize the rhythms, e.g. extending z=7 to z=14.

In a preferred embodiment, the checkpoint inhibitor may be an anti-PD-1 antibody, an anti-PD-L1 antibody, an anti-PD-L2 antibody, an anti-LAG3, an anti-TIM-3, an anti-CTLA4 antibody or an anti-TIGIT antibody, preferably an anti-PD-L1 antibody or an anti-PD-1 antibody. These antibodies have in common that they block/antagonize cellular interactions that block or downregulate immune cells, especially T cells from killing cancer cells, accordingly these antibodies are all antagonistic antibodies. Examples of anti-PD-1 antibodies are pembrolizumab, nivolumab, cemiplimab (REGN2810), BMS-936558, SHR1210, 1B1308, PDR001, BGB-A317, BCD-100 and JS001; examples of anti-PD-L1 antibodies are avelumab, atezolizumab, durvalumab, KN035 and MGD013 (bispecific for PD-1 and LAG-3); an example for PD-L2 antibodies is sHIgM12; examples of anti-LAG-3 antibodies are relatlimab (BMS 986016), Sym022, REGN3767, TSR-033, GSK2831781, MGD013 (bispecific for PD-1 and LAG-3) and LAG525 (IMP701); examples of anti-TIM-3 antibodies are TSR-022 and Sym023; examples of anti-CTLA-4 antibodies are ipilimumab and tremelimumab (ticilimumab); examples of anti-TIGIT antibodies are tiragolumab (MTIG7192A, RG6058) and etigilimab.

Especially preferred is the combination of the IL-2/IL-15Rβγ agonist, especially SO-C101, for use in the cyclic administration regimen with pembrolizumab. Presently, pembrolizumab is administered every 3 weeks. Accordingly, it is a preferred embodiment that the agonist is administered in a 3-week cycle as well, i.e. x is 7 days and repeated twice with y being 2, 3 or 4 days, and z is 7 days. In one embodiment, pembrolizumab is either administered at the first day of each treatment cycle as is the agonist, or at any other day within such treatment cycle, preferably at day 3, day 4 or day 5 of such treatment cycle in order to allow for an expansion/activation of NK cells and CD8⁺ T cells prior to the addition of the checkpoint inhibitor. In vitro experiments of present invention have shown that both concomitant and sequential treatment result in a marked increase of IFNγ production from PBMCs, showing. Recently, the label of pembrolizumab has been broadened to allow also for administration every 6 weeks. Compared to the schedules described in this section above, the schedule of the agonist would preferably adapted by either having two 3 week cycles (e.g. x=7 repeated once, z=7) or by having a 6 week cycle (e.g. x=7 repeated 4 times with z=7 or x=7 repeated 3 times with z=14).

In a preferred embodiment, the therapeutic antibody or tumor targeting antibody may be selected from an anti-CD38 antibody, an anti-CD19 antibody, an anti-CD20 antibody, an anti-CD30 antibody, an anti-CD33 antibody, an anti-CD52 antibody, an anti-CD79B antibody, an anti-EGFR antibody, an anti-HER2 antibody, an anti-VEGFR2 antibody, an anti-GD2 antibody, an anti-Nectin 4 antibody and an anti-Trop-2 antibody, preferably an anti-CD38 antibody. Such therapeutic antibody or tumor targeting antibody may be linked to a toxin, i.e. being an antibody drug conjugate. The therapeutic antibodies exert a direct cytotoxic effect on the tumor target cell through binding to the target expressed on the surface of the tumor cell. The therapeutic activity may be due to the receptor binding leading to modified signaling in the cell, antibody-dependent cellular cytotoxicity (ADCC), complement-dependent cytotoxicity (CDC) or other antibody-mediated killing of tumor cells. For example, the inventors have shown that the IL-2/IL-15Rβγ agonist RLI-15/SO-C101 synergizes with an anti-CD38 antibody (daratumumab) in tumor cell killing of Daudi cells in vitro both in a sequential and a concomitant setting, which was confirmed in a multiple myeloma model in vivo. Accordingly, anti-CD38 antibodies are especially preferred. Examples of anti-CD38 antibodies are daratumumab, isatuximab (SAR650984), MOR-202 (MOR03087), TAK-573 or TAK-079 or GEN1029 (HexaBody®-DR5/DR5), whereas most preferred is daratumumab. Preferably, daratumumab is administered according to its label, especially preferred via i.v. infusion and/or according to the dose recommended by its label, preferably at a dose of 16 mg/kg.

In a preferred embodiment, the IL-2/IL-15Rβγ agonist is for use, wherein an anti-CD38 antibody, preferably daratumumab, is administered in combination with the IL-2/IL-15Rβγ agonist, wherein (i) the anti-CD38 antibody is administered once a week for a first term of 8 weeks, (ii) followed by a second term consisting of 4 sections of 4 weeks (16 weeks), wherein during each 4 week section the anti-CD38 antibody is administered weekly in the first 2 weeks of the section followed by 2 weeks of no administration, (iii) followed by a third term with administration of the anti-CD38 antibody once every 4 weeks until disease progression. Therefore, it is preferred that the anti-CD38 antibody is administered once weekly for an initial 8 weeks, followed by 16 weeks of 2 treatments once per week and 2 weeks of treatment break, and thereafter once every 4 weeks until disease progression. Aligned to the treatment schedule of the IL-2/IL-15Rβγ agonist starting counting with day of the first treatment with the agonist, in weeks with anti-CD38 antibody administration, the anti-CD38 antibody is administered on the 1^{st} day (concomitant treatment) or the 3^{rd} day (sequential treatment) of the week. A treatment schedule with x=7 repeated once and z=14 matches with the first term of 8 weeks anti-CD38 treatment, followed by the second term with x=7 repeated once and z=14 and followed by the third term with x=7 repeated once and z=14. Alternatively, the agonist schedule may be x=7 repeated twice and z=7 to match the 4-week rhythm of the anti-CD38 antibody.

An example of an anti-CD19 antibody is Blinatumomab (bispecific for CD19 and CD3), for an anti-CD20 antibody are Ofatumumab and Obinutuzumab, an anti-CD30 antibody is Brentuximab, an anti-CD33 antibody is Gemtuzumab, for an anti-CD52 antibody is Alemtuzumab, an anti-CD79B antibody is Polatuzumab, for an anti-EGFR antibody is Cetuximab, an anti-HER2 antibody is Trastuzumab, an anti-VEGFR2 antibody is Ramucirumab, an anti-GD2 antibody is Dinutuximab, an anti-Nectin 4 antibody is Enfortumab and an anti-Trop-2 antibody is Sacituzumab.

Examples of aligned dosing schedules are the combination of SO-C101 with Ramucirumab, which is infused every 2 to 3 weeks depending on the indication. For a 3 week cycle of Ramucirumab, SO-C101 may be administered with x=7 repeated once and z=7. For two 2 week cycles of Ramucirumab, SO-C101 may be administered with x=7 repeated twice and z=7.

Dense Pulsed Dosing

In another aspect of the invention the IL-2/IL-15Rβγ agonist is for use according to the invention comprising administering the IL-2/IL-15Rβγ agonist to a human patient using a dense pulsed administration regimen, wherein the dense administration regimen comprises (“dense pulsed”):
(a) a first period of x days during which the IL-2/IL-15Rβγ agonist is administered at a daily dose on y consecutive days at the beginning of the first period followed by x-y days without administration of the IL-2/IL-15Rβγ agonist, wherein x is 5, 6, 7, 8, 9, 10, 11, 12, 13, 14, 15, 16, 17, 18, 19, 20 or 21 days, preferably, 7 or 14 days, and y is 2, 3 or 4 days, preferably 2 or 3 days;
(b) repeating the first period at least once; and
wherein the daily dose is split into 2 or 3 individual doses that are administered within one day, wherein the time interval between administration of the individual doses is at least about 4 h and preferably not more than 12 h.

Preferably, the administration regimen further comprises (c) a second period of z days without administration of the IL-2/IL-15Rβγ agonist (“dense pulsed cyclic”), wherein z is 5, 6, 7, 8, 9, 10, 11, 12, 13, 14, 15, 16, 17, 18, 19, 20, 21, 28, 35, 42, 49, 56, 63 or 70 days, preferably 7, 14, 21 or 56 days, more preferably 7 or 21 days.

It was shown that the same amount of the agonist—split into several doses and administered during the day—is more efficacious in stimulating NK cells and especially CD 8⁺ cells, the latter showing a lower sensitivity for the stimulation, than administered only in a single injection.

Such multiple dosing should be able to be integrated into the daily business of hospitals, doctor's practice or outpatient settings and therefore, 2 to 3 equal doses administered during business hours including shifts between 8 and 12 hours would still be conveniently manageable, with 8 or 10 h intervals being preferred as the maximum time difference between first and last dose. Accordingly, it is a preferred embodiment that the daily dose is split into 3 individual doses that are administered within one day, wherein the time interval between administration of the individual doses is about 5 to about 7 h, preferably about 6 hours. This means that a patient could be dosed e.g. at 7 am, 2 pm and 7 pm every day (with 6-hour intervals), or at 7 am, 1 pm and 6 pm (with 5 hour intervals). In another preferred embodiment, the daily dose is split into 2 individual doses that are administered within one day, wherein the time interval between administration of the individual doses is about 6 h to about 10 h, preferably 8 h. In the case of 2 doses, a patient could be dosed e.g. at 8 am and 4 pm (with an 8-hour interval). Given the daily routine of hospitals, the intervals between the administrations may vary within a day or from day to day. Surprisingly, in mice the same amount (about 40 μg/kg) of SO-C101 split into 3 doses (13 μg/kg) administered during the day lead to a drastic increase of CD8⁺ T cell counts as well as Ki67⁺ CD8 T cells as a measure for proliferating CD8⁺ T cells, and even have the amount split into 3×7 μg/kg still showed much higher expansion and activation of CD8⁺ T cells.

Accordingly, it is a preferred embodiment that the daily dose is split into 3 individual doses that are administered within one day, wherein the time interval between administration of the individual doses is about 5 to about 7 h, preferably about 6 hours. This means that a patient could be dosed e.g. at 7 am, 2 pm and 7 pm every day (with 6-hour intervals), or at 7 am, 1 pm and 6 pm (with 5 hour intervals). In another preferred embodiment, the daily dose is split into 2 individual doses that are administered within one day, wherein the time interval between administration of the individual doses is about 6 h to about 10 h, preferably 8 h. In the case of 2 doses, a patient could be dosed e.g. at 8 am and 4 pm (with an 8-hour interval). Given the daily routine of hospitals, the intervals between the administrations may vary within a day or from day to day.

The embodiments herein above for the pulsed cyclic dosing apply for the dense pulsed (and the dense pulsed cyclic dosing as a sub form of the dense pulsed dosing). This particularly applies to embodiments relating to the dose of the IL-2/IL-15Rβγ agonist to be administered, the way of administration (e.g., s.c. or i.p.), the effects on NK cell activation and NK cell numbers, the conditions to be treated, the half-life of the IL-2/IL-15Rβγ agonist, the IL-2/IL-15Rβγ agonist and the co-administration of checkpoint inhibitors.

Preferably, the IL-2/IL-15Rβγ agonist is for use in the dense pulsed or dense pulsed cyclic dosing regimen, wherein the daily dose is 0.1 μg/kg (0.0043 μM) to 50 μg/kg (2.15 μM), preferably 0.25 μg/kg (0.011 μM) to 25 μg/kg (1.1 μM), more preferably 0.6 μg/kg (0.026 μM) to 12 μg/kg (0.52 μM) and especially 2 μg/kg (0.087 μM) to 12 μg/kg (0.52 μM), preferably wherein the daily dose selected within the dose range of 0.1 μg/kg (0.0043 μM) to 50 μg/kg (2.15 μM) is not substantially increased during the administration regimen, preferably wherein the dose is maintained during the administration regimen.

In another embodiment, the dense pulsed dosing applies a daily dose, wherein the daily dose is a fixed dose independent of body weight of 7 μg to 3500 μg, preferably 17.5 μg to 1750 μg, more preferably 42 μg to 700 μg and especially 140 μg to 700 μg.

In another embodiment, the dense pulsed dosing applies daily doses, wherein the daily dose is increased during the administration regimen. Preferably, the daily dose is increased after each period of x days. In a further embodiment, the daily dose is increased by 20% to 100%, preferably by 30% to 50% after each period of x days.

In another embodiment, the daily dose is increased once after the first cycle. Preferably, the daily dose is increased by 20% to 100%, preferably by 30% to 50% after the first cycle.

In another embodiment, of the dense pulsed dosing, the IL-2/IL-15Rβγ agonist is administered subcutaneously (s.c.) or intraperitoneally (i.p.), preferably s.c.

Preferably, as further described above, administration of the IL-2/IL-15Rβγ agonist in step (a) results in (1) an increase of the % of Ki-67⁺ NK of total NK cells in comparison to no administration of the IL-2/IL-15Rβγ agonist, and wherein administration of the IL-2/IL-15Rβγ agonist in step (b) results in a Ki-67⁺ NK cell level that is at least 70% of the of the Ki-67⁺ NK cells of step (a), or (2) maintenance of NK cell numbers or preferably an increase of NK cell numbers to at least 110% as compared to no administration of IL-2/IL-15Rβγ agonist after at least one repetition of the first period, preferably after at least two repetitions of the first period, and/or (3) NK cell numbers of at least 1.1×10³ NK cells/μl after at least one repetition of the first period, preferably after at least two repetitions of the first period.

It is further preferred for the dense pulsed cyclic dosing that the cyclic administration is repeated over at least 5 cycles, preferably 8 cycles, more preferably at least 15 cycles and even more preferably until disease progression.

In another embodiment for the dense pulsed dosing regimen the IL-2/IL-15Rβγ agonist has an in vivo half-life of 30 min to 24 h, preferably 1 h to 12 h, more preferably of 2 h to 6 h.

In another embodiment for the dense pulsed dosing regimen, the IL-2/IL-15Rβγ agonist is an interleukin 15 (IL-15)/interleukin-15 receptor alpha (IL-15Rα) complex, preferably a fusion protein comprising the human IL-15Rα sushi domain or derivative thereof, a flexible linker and the human IL-15 or derivative thereof, preferably wherein the human IL-15Rα sushi domain comprises the sequence of SEQ ID NO: 6, and wherein the human IL-15 comprises the sequence of SEQ ID NO: 4, more preferably wherein the IL-15/IL-15Rα complex is SEQ ID NO: 9.

Further, IL-2/IL-15Rβγ agonist for use in the dense pulsed dosing may be administered in combination with a further therapeutic agent. Preferably, the further therapeutic agent and the IL-2/IL-15Rβγ agonist are administered on the same days and/or on different days. Further it is preferred that the administration of the further therapeutic agent occurs according to an administration regimen that is independent of the administration regimen of the IL-2/IL-15Rβγ agonist.

In one embodiment of the dense pulsed dosing regimen, the further therapeutic agent is selected from a checkpoint inhibitor or a therapeutic antibody.

Preferably, the checkpoint inhibitor is selected from an anti-PD-1 antibody, an anti-PD-L1 antibody, an anti-PD-L2 antibody, an anti-LAG-3 antibody, an anti-TIM-3 antibody, an anti-CTLA4 antibody or an anti-TIGIT antibody, preferably an anti-PD-L1 antibody or an anti-PD-1 antibody.

And preferably, the therapeutic antibody is selected from an anti-CD38 antibody, an anti-CD19 antibody, an anti-CD20 antibody, an anti-CD30 antibody, an anti-CD33 antibody, an anti-CD52 antibody, an anti-CD79B antibody, an anti-EGFR antibody, an anti-HER2 antibody, an anti-VEGFR2 antibody, an anti-GD2 antibody, an anti-Nectin 4 antibody and an anti-Trop-2 antibody, preferably an anti-CD38 antibody, preferably an anti-CD38 antibody.

Another embodiment of the present invention is a kit of parts comprising several doses of the IL-2/IL-15Rβγ agonist of the invention, an instruction for administration of such IL-2/IL-15Rβγ agonist in the cyclic administration regimens according to any embodiment above and optionally an administration device for the IL-2/IL-15Rβγ agonist.

Another embodiment of the present invention is a kit of parts comprising several doses of the IL-2/IL-15Rβγ agonist of the invention, an instruction for administration of such IL-2/IL-15Rβγ agonist in the pulsed administration regimens according to any embodiment above and optionally an administration device for the IL-2/IL-15Rβγ agonist.

Another embodiment of the present invention is a kit of parts comprising several doses of the IL-2/IL-15Rβγ agonist of the invention, an instruction for administration of such IL-2/IL-15Rβγ agonist in the dense pulsed administration regimens according to any embodiment above and optionally an administration device for the IL-2/IL-15Rβγ agonist.

Another embodiment is the use of an IL-2/IL-15Rβγ agonist in the manufacture of a kit of parts for the treatment of cancer, wherein the kit of parts comprises:
several doses of the IL-2/IL-15Rβγ agonist of the invention, an instruction for administration of such IL-2/IL-15Rβγ agonist in the cyclic administration regimen according to any embodiment above and optionally an administration device for the IL-2/IL-15Rβγ agonist.

Another embodiment is the use of an IL-2/IL-15Rβγ agonist in the manufacture of a kit of parts for the treatment of cancer, wherein the kit of parts comprises:
several doses of the IL-2/IL-15Rβγ agonist of the invention, an instruction for administration of such IL-2/IL-15Rβγ agonist in the pulsed administration regimen according to any embodiment above and optionally an administration device for the IL-2/IL-15Rβγ agonist.

Another embodiment is the use of an IL-2/IL-15Rβγ agonist in the manufacture of a kit of parts for the treatment of cancer, wherein the kit of parts comprises:
several doses of the IL-2/IL-15Rβγ agonist of the invention, an instruction for administration of such IL-2/IL-15Rβγ agonist in the dense pulsed administration regimen according to any embodiment above and optionally an administration device for the IL-2/IL-15Rβγ agonist.

In a preferred embodiment the kit further comprises a checkpoint inhibitor and an instruction for use of the checkpoint inhibitor or the therapeutic antibody. The invention also involves methods of treatment involving the above described pulsed cyclic and dense pulsed dosing regimens, as well as methods for stimulating NK cells and/or CD8⁺ T cells involving the above described pulsed cyclic, and dense pulsed dosing regimens.

Dense Dosing

In another aspect of the invention an interleukin-2/interleukin-15 receptor βγ (IL-2/IL-15Rβγ) agonist is for use in treating or managing cancer, comprising administering the IL-2/IL-15Rβγ agonist to a human patient using a dense administration regimen, wherein the dense administration regimen comprises administering a daily dose to a patient, wherein the daily dose is split into 2 or 3 individual doses that are administered within one day, wherein the time interval between administration of the individual doses is at least about 4 h and preferably not more than 12 h.

The time interval between administration of the individual doses may be as described for the above embodiments. The amount of the IL-2/IL-15Rβγ agonist may also be as described for the above embodiments.

### FIGURES

FIG. 1: Dosing schedule of first-in-human clinical trial. * 1 day; DLT dose-limiting toxicity;

(A) Part A: SO-C101 dosing schedule

(B) Part B: SO-C101 in combination with pembrolizumab dosing schedule.

FIG. 2: (A) photograph of skin squamous cell carcinoma of 62 year old female patient at screening of patient; (B) CT scan of respective area of A; (C) photograph of skin squamous cell carcinoma (SSCC) of patient after 4 cycles/12 weeks of SO-C101 monotherapy; (D) CT scan of respective area of C; (E) top panel: photographs of SSCC at screening (left, Jun. 3, 2020) and during treatment with SO-C101 (Jul. 3, 2020, Sep. 2, 2020, Sep. 23, 2020 and Oct. 14, 2020); bottom panel: photographs of SSCC at beginning of combination therapy of SO-C101 with pembrolizumab (Nov. 25, 2020) and during combination therapy (Dec. 15, 2020, Jan. 14, 2021). (F) to (M) Immune histochemistry of biopsies taken prior to SO-C101 treatment (baseline—panels F, G, H, I) or after SO-C101 treatment (at week 18—panels J, K, L, M). Panels F and J: stained for hematoxylin & eosin; panels G and K: stained for CD8; panels H and L: stained for PD-L1/CD8; panels I and M: stained for NKp46.

FIG. 3: Immune histochemistry of biopsies from thyroid gland carcinoma patient taken prior to SO-C101/pembrolizumab treatment (baseline—panels A, B, C, D) or after SO-C101/pembrolizumab treatment (at week 6—panels E, F, G, H). Panels A and E: stained for hematoxylin & eosin; panels B and F: stained for CD8; panels C and G: stained for PD-L1/CD8; panels D and H: stained for NKp46.

FIG. 4: photograph of skin squamous cell carcinoma of 74 year old female patient at screening of patient (Mar. 18, 2021) and after 2 cycles of combination therapy with SO-C101 at 6 μg/kg and 200 mg pembrolizumab (May 6, 2021).

FIG. 5: Immune histochemistry of biopsies from anal squamous cell carcinoma patient taken prior to SO-C101/pembrolizumab treatment (baseline—panels A, B, C, D) or after SO-C101/pembrolizumab treatment (at week 6—panels E, F, G, H). Panels A and E: stained for hematoxylin & eosin; panels B and F: stained for CD8; panels C and G: stained for PD-L1/CD8; panels D and H: stained for NKp46.

FIG. 6: Graphical representation of the pulsed cyclic administration regimens. 0 depicts cyclic dosing without an increase of the initial daily dose. A to E depict various scenarios of an increase of the daily dose: A—after the first treatment period x of each treatment cycle, whereas each treatment cycle starts again at the initial dose; B—after each treatment period x of each treatment cycle, whereas the daily dose is not increased after the break z; C—after each day of treatment within each treatment period x, wherein each treatment cycle starts again at the initial dose; D—after each day of treatment within each treatment period x, wherein the daily dose is not increased from one treatment period x to the next within a cycle and wherein each treatment cycle starts again at the initial dose; E—after each day of treatment within each treatment period x, wherein the daily dose is not increased from one treatment period x to the next within a cycle and wherein the daily dose of the first treatment period x of a new cycle starts at the daily dose of day 1 of the previous treatment period x.

FIG. 7: Increased proliferation of CD8⁺ T cells and NK cells following treatment with SO-C101 and SO-C101 and pembrolizumab in peripheral blood. (A) % Ki-67⁺ CD8⁺ T cells and (B) % Ki-67⁺ NK cells in dependence of SO-C101 dose levels from 0.25 to 15 μg/kg SO-C101 monotherapy and 1.5 to 5 μg/kg SO-C101 combination therapy with pembrolizumab. Clinically responsive patients (PR or ≥2SD) are marked with #.

FIG. 8: Increased density of CD3⁺ and CD8⁺ T cells and increased ratio of CD8⁺ T cells/Treg upon treatment with SO-C101 and SO-C101 and pembrolizumab in tumor tissue. (A) CD3⁺ T cell density in cells/mm² in tumor tissue, (B) CD8⁺ T cell density in cells/mm² in tumor tissue, and (C) CD8⁺ T cell/T_{reg} ratio in tumor tissue, in dependence of SO-C101 dose levels from 0.25 to 15 μg/kg SO-C101 monotherapy and 1.5 to 5 μg/kg SO-C101 combination therapy with pembrolizumab. Clinically responsive patients (PR or ≥2SD) are marked with #.

FIG. 9: SO-C101 induces genes involved in T cells and NK cell activation and immune-mediated tumor regression. (A) Immunosign® 21 gene signature score (HalioDx) profiling pre-defined set of genes reflecting T cell activation, attraction, cytotoxicity and T cell orientation, (B) expression of genes linked to antigen processing and presentation, and (C) expression of genes linked to NK cell functions. Each dot represents a different patient. Out of 18 patients, 15 were treated with SO-101 monotherapy (in black), 3 were treated with SO-C101 in combination with pembrolizumab (in grey). Clinically responsive patients (PR or ≥2SD) are marked with #.

| Sequences | | |
|---|---|---|
| human IL-2 | | |
| SEQ ID NO: 1 | | |
| 1 | MYRMQLLSCI ALSLALVINS APTSSSTKKT QLQLEHLLLD LQMILNGINN YKNPKLTRML | |
| 61 | TFKFYMPKKA TELKHLQCLE EELKPLEEVL NLAQSKNFHL RPRDLISNIN VIVLELKGSE | |
| 121 | TTFMCEYADE TATIVEFLNR WITFCQSIIS TLT | 153 |
| mature human IL-2 | | |
| SEQ ID NO: 2 | | |
| | APTSSSTKKT QLQLEHLLLD LQMILNGINN YKNPKLTRML | |
| 6 | TFKFYMPKKA TELKHLQCLE EELKPLEEVL NLAQSKNFHL RPRDLISNIN VIVLELKGSE | |
| 121 | TTFMCEYADE TATIVEFLNR WITFCQSIIS TLT | 153 |
| human IL-15 | | |
| SEQ ID NO: 3 | | |
| 1 | MRISKPHLRS ISIQCYLCLL LNSHELTEAG IHVFILGCFS AGLPKTEANW VNVISDLKKI | |
| 061 | EDLIQSMHID ATLYTESDVH PSCKVTAMKC FLLELQVISL ESGDASIHDT VENLIILANN | |
| 121 | SLSSNGNVTE SGCKECEELE EKNIKEFLQS FVHIVQMFIN TS | 162 |
| mature human IL-15 | | |
| SEQ ID NO: 4 | | |
| | NW VNVISDLKKI | |
| 061 | EDLIQSMHID ATLYTESDVH PSCKVTAMKC FLLELQVISL ESGDASIHDT VENLIILANN | |
| 121 | SLSSNGNVTE SGCKECEELE EKNIKEFLQS FVHIVQMFIN TS | 162 |
| human IL-15Ra | | |
| SEQ ID NO: 5 | | |
| 1 | MAPRRARGCR TLGLPALLLL LLLRPPATRG ITCPPPMSVE HADIWVKSYS LYSRERYICN | |
| 61 | SGFKRKAGTS SLTECVLNKA TNVAHWTTPS LKCIRDPALV HQRPAPPSTV TTAGVTPQPE | |
| 121 | SLSPSGKEPA ASSPSSNNTA ATTAAIVPGS QLMPSKSPST GTTEISSHES SHGTPSQTTA | |
| 181 | KNWELTASAS HQPPGVYPQG HSDTTVAIST STVLLCGLSA VSLLACYLKS RQTPPLASVE | |
| 241 | MEAMEALPVT WGTSSRDEDL ENCSHHL | |
| sushi domain of IL-15Rα | | |
| SEQ ID NO: 6 | | |
| CPPPMSVE HADIWVKSYS LYSRERYICN SGFKRKAGTS SLTECVLNKA TNVAHWTTPS LKC | | |
| sushi+ fragment of IL-15Rα | | |
| SEQ ID NO: 7 | | |
| ITCPPPMSVE HADIWVKSYS LYSRERYICN SGFKRKAGTS SLTECVLNKA TNVAHWTTPS | | |
| LKCIRDPALV HQRPAPP | | |
| linker | | |
| SEQ ID NO: 8 | | |
| SGG SGGGGSGGGS GGGGSGG | | |
| SO-C101 (RLI2) | | |
| SEQ ID NO: 9 | | |
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNGNVTES | |
| 181 | GCKECEELEE KNIKEFLQSF VHIVQMFINT S | 211 |
| IL2v | | |
| SEQ ID NO: 10 | | |
| 1 | APASSSTKKT QLQLEHLLLD LQMILNGINN YKNPKLTRML | |
| 41 | TAKFAMPKKA TELKHLQCLE EELKPLEEVL NGAQSKNFHL RPRDLISNIN VIVLELKGSE | |
| 101 | TTFMCEYADE TATIVEFLNR WITFAQSIIS TLT | |
| Leader peptide of (IL-15_{N72D})₂:IL-15Rαₛᵤₛₕᵢ-Fc: | | |
| SEQ ID NO: 11 | | |
| | METDTLLLWV LLLWVPGSTG | |
| IL-15Rαₛᵤₛₕᵢ (65aa)-Fc (IgG1 CH2-CH3): | | |
| SEQ ID NO: 12 | | |
| 1 | ITCPPPMSVE HADIWVKSYS LYSRERYICN SGFKRKAGTS SLTECVLNKA TNVAHWTTPS | |
| 61 | LKCIREPKSC DKTHTCPPCP APELLGGPSV FLFPPKPKDT LMISRTPEVT CVVVDVSHED | |
| 120 | PEVKFNWYVD GVEVHNAKTK PREEQYNSTY RVVSVLTVLH QDWLNGKEYK CKVSNKALPA | |
| 180 | PIEKTISKAK GQPREPQVYT LPPSRDELTK NQVSLTCLVK GFYPSDIAVE WESNGQPENN | |
| 240 | YKTTPPVLDS DGSFFLYSKL TVDKSRWQQG NVFSCSVMHE ALHNHYTQKS LSLSPGK | |
| IL-15_{N72D} | | |
| SEQ ID NO: 13 | | |
| | NW VNVISDLKKI | |
| 061 | EDLIQSMHID ATLYTESDVH PSCKVTAMKC FLLELQVISL ESGDASIHDT VENLIILAND | |
| 121 | SLSSNGNVTE SGCKECEELE EKNIKEFLQS FVHIVQMFIN TS | |

soluble IL-15Rα
SEQ ID NO: 14
MAPRRARGCRTLGLPALLLLLLLRPPATRGITCPPPMSVEHADIWVKSYSLYSRERYICN
SGFKRKAGTSSLTECVLNKATNVAHWTTPSLKCIRDPALVHQRPAPPSTVTTAGVTPQPE
SLSPSGKEPAASSPSSNNTAATTAAIVPGSQLMPSKSPSTGTTEISSHESSHGTPSQTTA
KNWELTASASHQPPGVYPQGHSDTT
IL-15_{L52C}
SEQ ID NO: 15
NWVNVISDLKKIEDLIQSMHIDATLYTESDVHPSCKVTAMKCFLLELQVISCESGDASIH
DTVENLIILANNSLSSNGNVTESGCKECEELEEKNIKEFLQSFVHIVQMFINTS
IL-15Rα-sushi+_{S40C}-Fc
SEQ ID NO: 16
ITCPPPMSVE HADIWVKSYS LYSRERYICN SGFKRKAGTC SLTECVLNKA TNVAHWTTPS
LKCIRDPALV HQRGGGGSGG GGSEPKSSDK THTCPPCPAP ELLGGPSVFL FPPKPKDTLM
ISRTPEVTCV VVDVSHEDPE VKFNWYVDGV EVHNAKTKPR EEQYNSTYRV VSVLTVLHQD
WLNGKEYKCK VSNKALPAPI EKTISKAKGQ PREPQVYTLP PSREEMTKNQ VSLTCLVKGF
YPSDIAVEWE SNGQPENNYK TTPPVLDSDG SFFLYSKLTV DKSRWQQGNV FSCSVMHEAL
HNHYTQKSLS LSPGK
NEO 2-15 E62C
SEQ ID NO: 17
PKKKIQLHAEHALYDALMILNIVKTNSPPAEEKLEDYAFNFELILEEIARLFESGDQKDE
ACKAKRMKEWMKRIKTTASEDEQEEMANAIITILQSWIFS
XENP024306 chain 1: human IL-15 D₃₀N/E₆₄Q/N₆₅D (GGGGS)₁-
Fc(216)_IgG1_pI(−) Isosteric A C2205/PVA_/S₂₆₇K/L₃₆₈D/K₃₇₀S/M₄₂₈L/N₄₃₄S
SEQ ID NO: 18
NWVNVISDLKKIEDLIQSMHIDATLYTESNVHPSCKVTAMKCFLLELQVISLESGDASIH
DTVQDLIILANNSLSSNGNVTESGCKECEELEEKNIKEFLQSFVHIVQMFINTSGGGGSE
PKSSDKTHTCPPCPAPPVAGPSVFLFPPKPKDTLMISRTPEVTCVVVDVKHEDPEVKENW
YVDGVEVHNAKTKPREEEYNSTYRVVSVLTVLHQDWLNGKEYKCKVSNKALPAPIEKTIS
KAKGQPREPQVYTLPPSREEMTKNQVSLTCDVSGFYPSDIAVEWESDGQPENNYKTTPPV
LDSDGSFFLYSKLTVDKSRWEQGDVFSCSVLHEALHSHYTQKSLSLSPGK
XENP024306 chain 2: human IL 15Rα(sushi) (GGGGS)₁-
Fc(216)_IgG1_C2205/PVA_/S₂₆₇K/S₃₆₄K/E₃₅₇Q/M₄₂₈L/N₄₃₄S
SEQ ID NO: 19
ITCPPPMSVEHADIWVKSYSLYSRERYICNSGFKRKAGTSSLTECVLNKATNVAHWTTPS
LKCIRGGGGSEPKSSDKTHTCPPCPAPPVAGPSVFLFPPKPKDTLMISRTPEVTCVVVDV
KHEDPEVKFNWYVDGVEVHNAKTKPREEQYNSTYRVVSVLTVLHQDWLNGKEYKCKVSNK
ALPAPIEKTISKAKGQPREPQVYTLPPSREQMTKNQVKLTCLVKGFYPSDIAVEWESNGQ
PENNYKTTPPVLDSDGSFFLYSKLTVDKSRWQQGNVFSCSVLHEALHSHYTQKSLSLSPG
K

The invention is further described by the following embodiments:

The IL-2/IL-15Rβγ agonist for the use as described herein, wherein the daily dose of the IL-2/IL-15Rβγ agonist is 0.1 μg/kg to 50 μg/kg, preferably 0.25 μg/kg to 25 μg/kg, more preferably 0.6 μg/kg to 12 μg/kg and even more preferably 2 μg/kg to 12 μg/kg, preferably 3 μg/kg to 20 μg/kg, more preferably 6 to 12 μg/kg.

The IL-2/IL-15Rβγ agonist for the use as described herein, wherein the daily dose selected within the dose range of 0.1 to 50 μg/kg is not substantially increased during the administration regimen, preferably wherein the dose is maintained during the administration regimen.

The IL-2/IL-15Rβγ agonist for the use as described herein, wherein the daily dose is a fixed dose independent of body weight of 7 μg to 3500 μg, preferably 17.5 μg to 1750 μg, more preferably 42 μg to 700 μg and especially 140 μg to 700 μg.

The IL-2/IL-15Rβγ agonist for the use as described herein, wherein the daily dose is increased during the administration regimen.

The IL-2/IL-15Rβγ agonist for the use as described herein, wherein the daily dose is increased after each period of x days.

The IL-2/IL-15Rβγ agonist for the use as described herein, wherein the daily dose is increased by 20% to 100%, preferably by 30% to 50% after each period of x days.

The IL-2/IL-15Rβγ agonist for the use as described herein, wherein the daily dose is increased once after the first period of x days.

The IL-2/IL-15Rβγ agonist for the use as described herein, wherein the daily dose is increased by 20% to 100%, preferably by 30% to 50% after the first period of x days.

The IL-2/IL-15Rβγ agonist for the use as described herein wherein the daily dose is administered in a single injection.

The IL-2/IL-15Rβγ agonist for the use as described herein, wherein the daily dose is split into 2 or 3 individual doses that are administered within one day, wherein the time interval between administration of the individual doses is at least about 4 h and preferably not more than 14 h.

The IL-2/IL-15Rβγ agonist for the use as described herein, wherein the daily dose is split into 3 individual doses that are administered within one day, wherein the time interval between administration of the individual doses is about 5 to about 7 h, preferably about 6 h.

The IL-2/IL-15Rβγ agonist for the use as described herein, wherein the daily dose is split into 2 individual doses that are administered within one day, wherein the time interval between administration of the individual doses is about 6 h to about 10 h, preferably about 8 h.

The IL-2/IL-15Rβγ agonist for the use as described herein, wherein the IL-2/IL-15Rβγ agonist is administered subcutaneously (s.c.) or intraperitoneally (i.p.), preferably s.c.

The IL-2/IL-15Rβγ agonist for the use as described herein, wherein administration of the IL-2/IL-15Rβγ agonist in step (a) results in
(1) an increase of the % of Ki-67⁺ NK of total NK cells in comparison to no administration of the IL-2/IL-15Rβγ agonist, and wherein administration of the IL-2/IL-15Rβγ agonist in step (b) results in a Ki-67⁺ NK cell level that is at least 70% of the of the Ki-67⁺ NK cells of step (a), or
(2) maintenance of NK cell numbers or preferably an increase of NK cell numbers to at least 110% as compared to no administration of IL-2/IL-15Rβγ agonist after at least one repetition of the first period, preferably after at least two repetitions of the first period, and/or
(3) NK cell numbers of at least 1.1×10³ NK cells/μl after at least one repetition of the first period, preferably after at least two repetitions of the first period.

The IL-2/IL-15Rβγ agonist for the use as described herein, wherein the cyclic administration is repeated over at least 3 cycles, preferably 5 cycles, more preferably at least 10 cycles and even more preferably until disease progression.

The IL-2/IL-15Rβγ agonist for use the use as described herein, wherein the IL-2/IL-15Rβγ agonist has an in vivo half-life of 30 min to 24 h, preferably 1 h to 12 h, more preferably of 2 h to 6 h.

The invention is also described the following items:

1. An interleukin-2/interleukin-15 receptor βγ (IL-2/IL-15Rβγ) agonist for use in the treatment of a HPV-induced tumor or a HPV-induced cancer in a human patient.

2. The IL-2/IL-15Rβγ agonist for the use of item 1, whereas the HPV-induced tumor or HPV-induced cancer is selected from the group consisting of cervical cancer, head-and-neck squamous cell carcinomas, oral neoplasias, oropharyngeal cancer (oropharynx squamous cell carcinoma), penile, anal, vaginal, vulvar cancers and HPV-associated skin cancers (e.g. skin squamous cell carcinoma or keratinocyte carcinoma).

3. The IL-2/IL-15Rβγ agonist for the use of item 1 or item 2, whereas the HPV-induced tumor or HPV-induced cancer is positive for one or more of HPV types 16, 18, 26, 31, 33, 35, 39, 45, 51, 52, 53, 56, 58, 59, 66, 68, 73 and 82, especially types 16, 18, 31, 33 and 45.

4. The IL-2/IL-15Rβγ agonist for the use of any one of items 1 to 3, whereas the patient is resistant or refractory to at least one immune checkpoint inhibitor treatment.

5. The IL-2/IL-15Rβγ agonist for the use of any one of items 1 to 4, wherein the IL-2/IL-15Rβγ agonist is not administered in combination with an immune checkpoint inhibitor.

6. The IL-2/IL-15Rβγ agonist for the use of any one of items 1 to 4, wherein the IL-2/IL-15Rβγ agonist is not administered in combination with a PD-1 antagonist.

7. The IL-2/IL-15Rβγ agonist for the use of item 4, wherein the IL-2/IL-15Rβγ agonist is not administered in combination with the immune checkpoint inhibitor the patient is refractory or resistant to, preferably wherein the immune checkpoint inhibitor the patient is refractory or resistant to and that is not administered in combination is a PD-1 antagonist.

8. The IL-2/IL-15Rβγ agonist for the use of any one of items 1 to 4, wherein the IL-2/IL-15Rβγ agonist is administered in combination with an immune checkpoint inhibitor.

9. The IL-2/IL-15Rβγ agonist for the use of any one of items 1 to 4 and 8, wherein the IL-2/IL-15Rβγ agonist is administered in combination with a PD-1 antagonist.

10. The IL-2/IL-15Rβγ agonist for the use of any one of items 4, 8 and 9, wherein the IL-2/IL-15Rβγ agonist is administered in combination with the immune checkpoint inhibitor the patient is refractory or resistant to, preferably wherein the immune checkpoint inhibitor the patient is refractory or resistant to and that is administered in combination is a PD-1 antagonist.

11. The IL-2/IL-15Rβγ agonist for the use of any one of items 1 to 10, wherein the treatment of the HPV-induced tumor results in at least about 30% size reduction of the tumor present prior to the treatment, preferably about 30% size reduction within 16 weeks of the treatment, preferably about 50% size reduction within 16 weeks of the treatment.

12. The IL-2/IL-15Rβγ agonist for the use of any one of items 1 to 11, wherein the response to the IL-2/IL-15Rβγ agonist is mediated by the innate immune response mediated by NK cells.

13. The IL-2/IL-15Rβγ agonist for the use of any one of items 1 to 12, whereas the IL-2/IL-15Rβγ agonist is administered according to a cyclical administration regimen, wherein the cyclical administration regimen comprises:
(a) a first period of x days during which the IL-2/IL-15Rβγ agonist is administered at a daily dose on y consecutive days at the beginning of the first period followed by x-y days without administration of the IL-2/IL-15Rβγ agonist, wherein x is 5, 6, 7, 8, 9, 10, 11, 12, 13, 14, 15, 16, 17, 18, 19, 20 or 21 days, preferably, 7 or 14 days, and y is 2, 3 or 4 days, preferably 2 or 3 days;
(b) repeating the first period at least once; and
(c) a second period of z days without administration of the IL-2/IL-15Rβγ agonist, wherein z is 5, 6, 7, 8, 9, 10, 11, 12, 13, 14, 15, 16, 17, 18, 19, 20, 21, 28, 35, 42, 49, 56, 63 or 70 days, preferably 7, 14, 21 or 56 days, more preferably 7, 14 or 21 days.

14. The IL-2/IL-15Rβγ agonist for the use of item 13, wherein x is 7 days, y is 2, 3 or 4 days and z is 7 days, preferably wherein y is 2 days and z is 7 days.

15. The IL-2/IL-15Rβγ agonist for the use of any one of items 1 to 14, wherein the daily dose of the IL-2/IL-15Rβγ agonist is 0.1 μg/kg to 50 μg/kg, preferably 0.25 μg/kg to 25 μg/kg, more preferably 0.6 μg/kg to 12 μg/kg and even more preferably 2 μg/kg to 12 μg/kg, preferably 3 μg/kg to 20 μg/kg, more preferably 6 to 12 μg/kg.

16. The IL-2/IL-15Rβγ agonist for the use of any one of items 1 to 15, wherein the IL-2/IL-15Rβγ agonist is an interleukin 15 (IL-15)/interleukin-15 receptor alpha (IL-15Rα) complex, preferably a fusion protein comprising the human IL-15Rα sushi domain or derivative thereof, a flexible linker and the human IL-15 or derivative thereof, preferably wherein the human IL-15Rα sushi domain comprises the sequence of SEQ ID NO: 6, and wherein the human IL-15 comprises the sequence of SEQ ID NO: 4, more preferably wherein the IL-15/IL-15Rα complex is SEQ ID NO: 9.

In a further embodiment, methods of treatment with the IL-2/IL-15Rβγ agonists as defined in the specification are included.

The following examples are to be construed as merely illustrative, and not limitative of the remainder of the disclosure in any way. All publications cited herein are incorporated by reference for the purpose or subject matter referenced herein.

### Examples

1. Clinical Trial of RLI-15/SO-C101

A first-in-human multicenter open-label phase 1/1b study to evaluate the safety and preliminary efficacy of SO-C101 as monotherapy and in combination with pembrolizumab in patients with selected advanced/metastatic solid tumors in ongoing (EurdraCT number 2018-004334-15, Clinicaltrials.gov number NCT04234113). RLI-15 is administered s.c. at a starting dose of 0.25 μg/kg and up to 48 μg/kg on days 1, 2, 8 and 9. In the combination part of the clinical trial RLI-15 will be combined with Keytruda© 25 mg/ml/pembrolizumab, which is administered i.v. at a dose of 200 mg q3w.

This study will assess the safety and tolerability of SO-C101 administered as monotherapy (Part A) and in combination with an anti-PD-1 antibody (pembrolizumab) (Part B) in patients with selected relapsed/refractory advanced/metastatic solid tumors (renal cell carcinoma, non-small cell lung cancer, small-cell lung cancer, bladder cancer, melanoma, Merkel-cell carcinoma, skin squamous-cell carcinoma, microsatellite instability high solid tumors, triple-negative breast cancer, mesothelioma, thyroid cancer, thymic cancer, cervical cancer, biliary track cancer, hepatocellular carcinoma, ovarian cancer, gastric cancer, head and neck squamous-cell carcinoma, and anal cancer), who are refractory to or intolerant of existing therapies known to provide clinical benefit for their condition.

Key inclusion criteria are:

Adults ≥18 years at screening; histologically or cytologically confirmed advanced and/or metastatic solid tumors who are refractory or intolerant to existing therapies; recovered from side effects from prior treatments to grade ≤1 toxicity; adequate hematological, cardiovascular, hepatic and renal functions; adequate laboratory parameters; accessible tumor tissue available for fresh biopsy; Eastern Cooperative Oncology Group (ECOG) Performance Status 0-1; measurable disease per iRECIST.

Key exclusion criteria are:

Patient with untreated CNS metastases and/or leptomeningeal carcinomatosis; any active autoimmune disease (AD) or history of syndrome that required systemic steroids (except of allowed doses) or immunosuppressive medication; prior exposure to the drugs that are agonist of IL-2 or IL-15; known HIV or active hepatitis B or C; uncontrolled hypertension (systolic >160 mm Hg and/or diastolic >110 mm Hg) or clinically significant cardiovascular disease, cerebrovascular accident/stroke, or myocardial infarction within 6 months prior to first study medication.

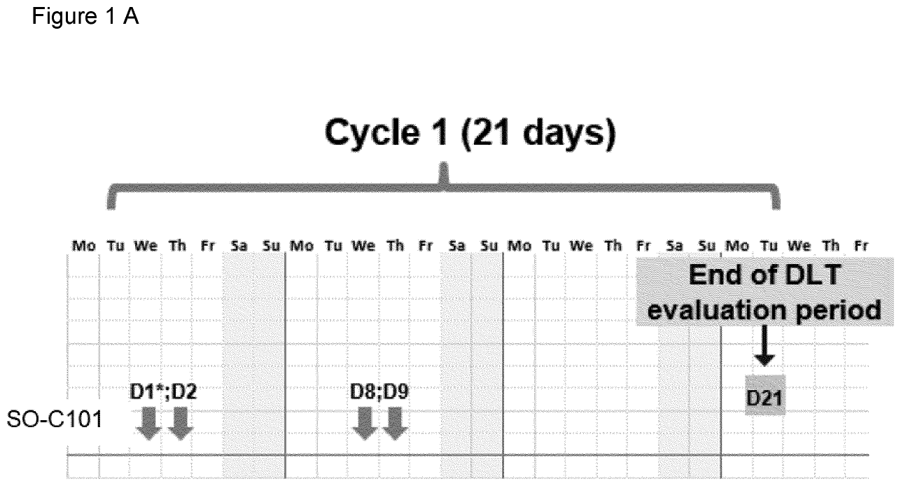

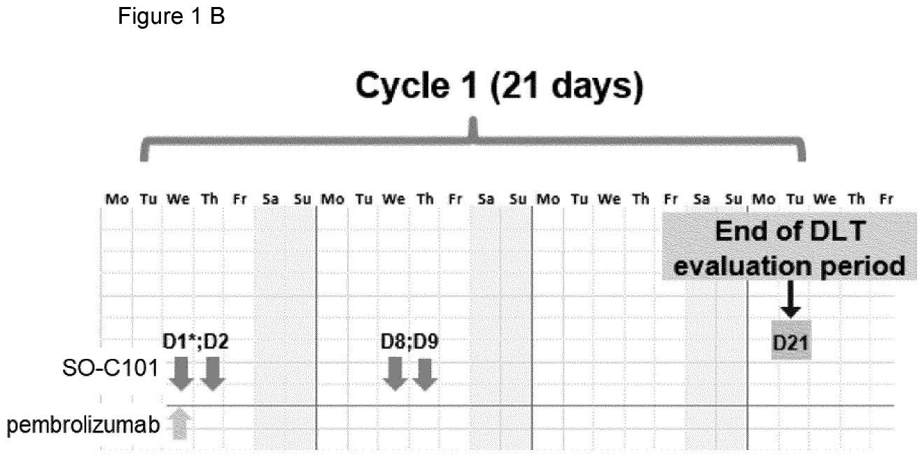

Part A started with an SO-C101 monotherapy dose escalation from 0.25 μg/kg administered s.c. and the MTD was reached at 15 μg/kg. The recommended phase 2 dose (RP2D) of SO-C101 monotherapy is defined at the dose level below 15 μg/kg, i.e. 12 μg/kg. Patients are treated with SO-C101 on day 1 (+1 day; Wednesday), day 2 (Thursday), day 8 (Wednesday), and day 9 (Thursday) of the 21-day cycle (FIG. 1A). The start of the treatment (day 1) is planned to be on a Wednesday as much as possible to allow biomarker sampling (fresh peripheral blood mononuclear cells [PBMCs] transfer to the central laboratory) on weekdays. However, as long as the two doses per week are given on consequent days (day 1 and day 2) and the second week dosing (day 8 and day 9) takes place 7 days after day 1, there will be ±1 day flexibility for the day 1 dosing to take place on a Tuesday or on a Thursday. Patients recruited in Part A will continue treatment at their assigned dose level. Patients will be discontinued from study treatment for any of the following events: (i) Radiographic disease progression; (ii) Clinical disease progression (investigator assessment); (iii) AE (inter-current illness or study treatment-related toxicity, including dose-limiting toxicities, that would, in the judgment of the investigator, affect assessments of clinical status to a significant degree or require discontinuation of study treatment)

The starting dose of Part B was 1.5 μg/kg SO-C101 administered as in Part A, which is combined with a fixed dose of pembrolizumab (200 mg i.v. every 3 weeks). Patients are to be treated with escalating doses of SO-C101 on day 1 (±1 day) (Wednesday), day 2 (Thursday), day 8 (Wednesday), and day 9 (Thursday) together with a fixed dose of pembrolizumab (200 mg i.v. every 3 weeks) given on the day 1 administration of SO-C101 (FIG. 1B). Pembrolizumab is administered within 30 minutes after the first dose of SO-C101 and as outlined in the package insert. The start of the treatment (day 1) is planned to be on a Wednesday as much as possible to allow biomarker sampling (fresh PBMCs transfer to the central laboratory) on weekdays. However, as long as the two doses of SO-C101 per week are given on consequent days (day 1 and day 2) and the second week SO-C101 dosing (day 8 and day 9) takes place 7 days after day 1, there will be ±1 day flexibility. Patients will continue SO-C101 and pembrolizumab treatment at the assigned dose level of SO-C101. In case SO-C101 needs to be stopped for reasons other than disease progression, pembrolizumab treatment could continue for up to 1 year as assessed by the DEC, if the patient does not progress and can tolerate the treatment. In case pembrolizumab needs to be stopped, SO-C101 treatment could continue until disease progression or unacceptable toxicity. Patients will be discontinued from study treatment for any of the following events: (i) Radiographic disease progression; (ii) Clinical disease progression (investigator assessment); (iii) AE (inter-current illness or study treatment-related toxicity, including dose-limiting toxicities, that would, in the judgment of the investigator, affect assessments of clinical status to a significant degree or require discontinuation of study treatment).

Preliminary Results

Part A enrollment started in July 2019 and MTD was reached at dose level 15 μg/kg. Thirty patients with a median of 3 (range 1-9) lines of previous systemic therapies were treated at dose levels 0.25, 0.75, 1.5, 3.0, 6.0, 9.0, 12.0, and 15 μg/kg BW. MTD at 15 μg/kg was defined due to 2 DLTs (increased liver function tests, quickly resolved after study drug discontinuation without sequelae). Indications of patients and best overall responses are shown in Table 2. Maximum level of NK cell activation was already reached at low dose levels and Maximum CD8⁺ T cell activation was reached at 9-12 μg/kg. Therefore the RP2D was selected to be 12 μg/kg. Safety data from 30 patients treated at 8 dose-levels indicate that SO-C101 monotherapy is well tolerated. The majority of AEs were fever, lymphopenia, local injection site reactions, chills, transaminase increases, flu-like symptoms as well as symptoms of cytokine release syndrome (mainly <Grade 2 except for lymphopenia). Lymphopenia is considered mode of action-related and usually resolved within a few days.

A partial response was seen in a 62 y female pt. with SSCC, previously CPI refractory. Long-lasting stable disease (SD) was observed in 3 patients:
71 y male pt. with Kidney cancer, 7 previous lines, CPI relapsed, SD for 93 days
47 y male pt. with NSCLC, 5 previous lines, CPI relapsed, SD for 155 days
57 y female pt. with Biliary tract carcinoma, 4 previous lines, CPI relapsed, SD for 148 days

Preliminary PK results showed the PK profile to be dose-proportional, with a Tₘₐₓ of approx. 5-6 hours after administration and a terminal half-life of approx. 4 hours.

Part B enrollment started in July 2020 and as of Oct. 8, 2021 fourteen patients with a median of 2 (range 1-6) lines of previous systemic therapies were treated at dose levels 1.5, 3.0, 6.0 and 9 μg/kg BW. Dose level 9 μg/kg is ongoing.

Patients were aged between 31 and 80 years at enrollment. The duration of the treatment ranged from 1 day to 393 days (as of Oct. 8, 2021). Indications of patients and best overall responses are shown in Table 3. SO-C101 in combination with pembrolizumab was well tolerated. The adverse event profile was consistent with the monotherapy AE profile from either single agent compound. Dose level 6 g/kg was expanded to 7 patients due to a DLT. The DLT was a cytokine release syndrome (CRS) grade 3 in one patient after the first administration. The patient continued the study on a reduced dose (3 μg/kg).

TABLE 2
Part A SO-C101 mono-treatment (cohort 1-8) - best overall
response (SD—stable disease, PR—partial response)

| Indication | Dose/μg/kg | clinical response |
|---|---|---|
| Gastric | 0.25 | none |
| Ovarian | | none |
| Gastro-esophageal | | none |
| Ovarian | 0.75 | 1 SD |
| Gastro-esophageal | | none |
| Kidney | | consent withdrawn |
| Melanoma | 1.5 | subjective benefit |
| Biliary tract | | 1 SD |
| Merkel cell | | none |
| Cervix uteri | 3 | none |
| Anal (epidermoid) | | none |
| Urothel. bladder | | none |
| Biliary tract | | 1 SD, consent withdrawn |
| Skin SCC | 6 | 1 SD, then 2 PD, treatment continued |
| | | with combination (outside of study) |
| Urothel. bladder | | none |
| Kidney | | 2 SD |
| Merkel cell | 9 | none |
| Kidney | | none |
| NSCLC | | 3 SD |
| Ovarian | | none |
| Biliary tract | 12 | 2 SD, treatment still ongoing |
| SCC (tonsil) | | 1 SD |
| Bladder | | none |
| Biliary tract | | 1 SD |
| Thymus | | no staging, treatment still ongoing |
| Thyroid gland | | none, treatment ongoing |
| SCC (eye canthus) | 15 | none |
| SCC (tonsil) | | 1 SD |
| NSCLC | | consent withdrawn |
| Merkel cell | | discontinued after adverse event |

TABLE 3
Part B SO-C101 + pembrolizumab combination
treatment (cohort 1-4, ongoing) - best overall
response (SD—stable disease, PR—partial response)

| | Dose/ | | CPI |
|---|---|---|---|
| Indication | μg/kg | clinical response | relapsed |
| Anal SCC | 1.5 | 7 SD | |
| Ampullary | | none | |
| Carcin | | | |
| Ovarian | | none | |
| Anal SCC | 3 | none | |
| Gastric | | 2 SD | |
| Thyroid gland | | 2 SD then 3 PR, treatment ongoing | |
| Skin SCC | 6 | 1 PR, treatment ongoing beyond | yes |
| | | progressive disease due to | |
| | | decrease in overall number of lesions | |
| Cervix uteri | | 2 SD, treatment ongoing | no |
| Urothel. bladder | | none | yes |
| Liver | | 2 SD, treatment ongoing | yes |
| Gastric | | 1 SD, treatment ongoing | no |
| Colorectal | | discontinued due to adverse event | yes |
| Skin melanoma | | 3 PR, treatment ongoing | yes |
| Cervical | 9 | no staging yet, treatment ongoing | |
| melanoma | | | |

2. Case Report of Patient with Skin Squamous Cell Carcinoma

A 62-year old female patient (race and ethnicity not reported) with skin squamous cell carcinoma was treated s.c. with SO-C101 at 6 μg/kg as monotherapy within the clinical study SC 103 part A (example 1, ICF version 5 and protocol version 5) starting with the first dose Jun. 4, 2020 (initially the clinical trial center erroneously reported 15 May 2020 as starting date; this has now been corrected) and monotherapy treatment was ongoing until Oct. 14, 2020.

In medical history there was appendectomy in the past and cerebral stroke in 2019, whereas all other medical history was connected to the disease under the study including fatigue, tumoral pain and anorexia. The initial diagnosis of squamous cell carcinoma of the skin was made in 2014 with known mutation/expression status p53, TERT. Initial surgery was performed in 2014 and the patient received radiotherapy as prior anticancer non-systemic therapy applying a dose of 66 Gray location to the tumor bed and a dose of 50 Gray to the left lymph node area of the ear.

The patient received 2 lines of previous systemic anticancer therapies: First line treatment with Docetaxel, Cisplatin, and Cetuximab (TPEx) was administered to the patient from March 2019 until June 2019. In second line treatment the patient received the anti-PD-1 immune check point inhibitor Cemiplimab, administered from 31 Jan. 2020 until 23-Apri1-2020. The patient relapsed upon the check point inhibitor treatment.

During the course of the study, there were a Grade 3 vasovagal reaction (not related to SO-C101) and dysphagia recorded. For dysphagia the patient received a nasogastric intubation, which is still ongoing as of September 18^{th}. Grade 2 anemia, fatigue and anorexia were reported, all other adverse events were Grade 1. No serious adverse events were reported.

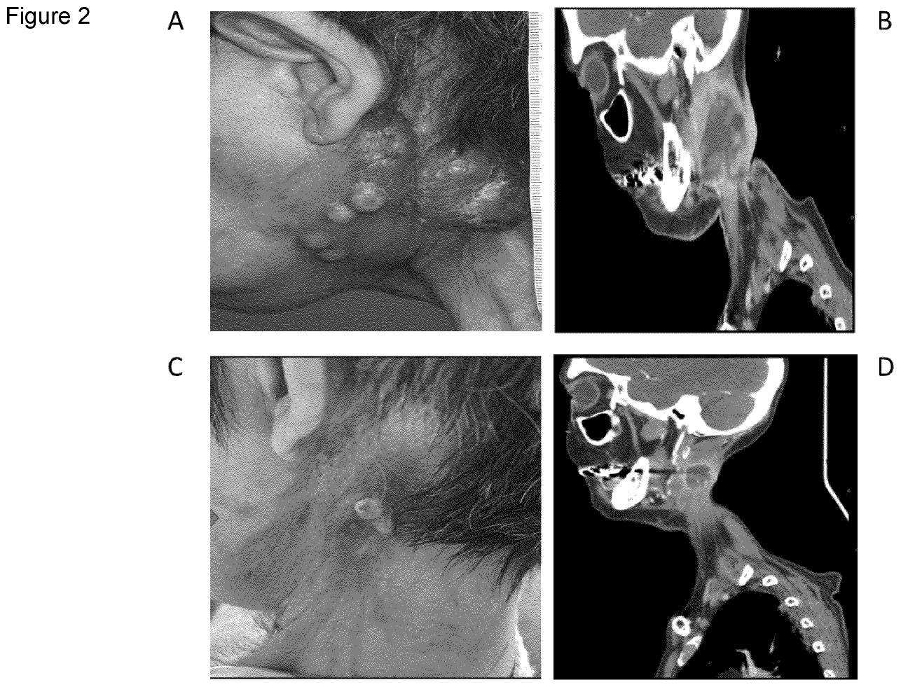

At the screening of the patient on Jun. 3, 2020, there was one target lesion present, nodal with diameter 50 mm, left cervical lymphadenopathy. Further, three non-target lesions were identified, all nodal, left and right cervical lymphadenopathy and liver segment III. A CT scan with contrast was used for tumor assessment. Treatment with SO-C101 was initiated on Jun. 4, 2020 with a daily dose of 6 μg/kg. A continuous improvement of the clinical response was observed over four cycles. The tumor assessment on Jul. 3, 2020 (at 4 cycles of SO-C101) revealed that the target lesion was reduced to 40 mm in diameter, equivalent to a disease reduction of 20%; the overall response was assessed as stable disease. At the third tumor assessment on 17 Aug. 2020 (at 12 weeks of SO-C101), a further shrinkage of the target lesion to 26 mm observed (see FIG. 2) equivalent of 49% reduction of the sum of the lesions; the overall response was accordingly assessed as partial response. As of September 18^{th}, the patient was receiving opioids and painkillers as concomitant treatment, nutritional support for dysphagia and medication for anemia and hypomagnesemia. After cycle 2 of treatment with SO-C101, there was a reduced need for opioids and pain killers.

Further tumor staging was performed on Oct. 2, 2020, with a further shrinkage of the target lesion to 21 mm thereby constituting a confirmed partial response (58% reduction), see FIG. 2 E. At the next tumor staging on Oct. 14, 2020, the patient showed tumor progression with a diameter of the target lesion of 37 mm (+76% compared to the previous staging). Monotherapy with SO-C101 was stopped due to progressive disease.

Surprisingly, monotherapy with SO-C101 lead to a partial response, duration over four months, with a 58% reduction of the target lesion in a terminally ill patient having skin squamous cell carcinoma, who has progressed after radiation therapy and two further lines of therapy, including the immune-oncology (10) drug Cemiplimab, an anti-PD-1 antibody.

The observed partial response went along with the observation of 71% of proliferating NK cells and 38% proliferating CD8⁺ T cells in blood.

The patient continued treatment with the combination of 1.5 μg/kg SO-C101 (according to the schedule of the monotherapy) and 200 mg pembrolizumab q3w on Nov. 26, 2020. Within 2 weeks, the patient again showed a clinical response with a marked reduction of the target lesion on photographs taken Dec. 15, 2020, and Jan. 14, 2021 (see FIG. 2 E). CT scans on Feb. 5 and Mar. 19, 2021 demonstrated for a 62% decrease from start of the study and 9% from nadir. A PET-CT from May 5, 2021 showed no “hot spot”, i.e. proliferating tumor.

Although the patient, before being treated with SO-C101, had relapsed under the treatment with cemiplimab (an anti-PD-1 antibody), and, after showing a confirmed partial response under SO-C101 monotherapy before presenting with further progressive disease, the patient again clinically responded significantly under the combination treatment of SO-C101 and pembrolizumab, another anti-PD-1 antibody. Accordingly, it surprisingly can be concluded that the SO-C101 monotherapy sensitized the tumor to be (again) responsive to anti-PD-1 treatment.

Infiltration of immune cells into the tumor was determined by immuno-histochemistry on tumor biopsies obtained at baseline and after SO-C101 EOT (week 18). Briefly, PD-L1 expression was determined using the Halioseek™ PD-L1/CD8 assay (Veracyte, France) with proprietary PD-L1 mAb (clone HDX3) and CD8 mAb (clone HDX1) on Ventana Benchmark XT. Detection of PD-L1 was performed with a secondary mAb using OptiView Universal DAB detection kit. Counterstaining was performed using Hematoxylin & Bluing Reagent. Slides were scanned with the NanoZoomer-XR to generate digital images (20×). CD8 and NKp46 expression was determined using Brightplex® multiplex IHC panel comprised of NKp46, Ki-67, CD8, CD3 and AE1/AE3. Following mAb were used: anti-NKp46 mAb cat.n. MOGI-M-H46-2/3, Veracyte; anti-Ki-67 mAb cat.n. HD-RM-000539/9027S, Veracyte/Cell Signaling; anti-CD8 mAb cat.n. HD-FG-000019, Veracyte); anti-CD3 mAb cat.n. HD-FG-000013, Veracyte; and anti-AE1/AE3 cat.n. HD-RM-000502/Sc81714, Santa Cruz. Briefly, Successive stainings were performed on the same slide using a Leica Bond RX. Signal detection was performed using MACH2 rabbit universal HRP polymer, MACH2 mouse universal HRP polymer or MACH4 mouse universal HRP polymer as secondary antibody and ImmPACT™ AMEC Red detection. Counterstaining of cellular nuclei using hematoxylin was performed at the end of the staining workflow. Slides were scanned with the Nanozoomer XR (×20). Each sample was analysed using HalioDx Digital Pathology Platform. Images were aligned with Brightplex®-fuse (in-house software).

TABLE 4
Infiltration of immune cells

| | CD8⁺ T cells | PD-L1⁺ cells | NKp46⁺ NK cells |
|---|---|---|---|
| | [cells/mm²] | [cells/mm²] | [cells/mm²] |
| baseline | 99 | 63 | 0.46 |
| Week 18 | 382 | 1731 | 19 |
| increase | ~4fold | ~30fold | ~40fold |

Prior to SO-C101 treatment, only a low infiltration of CD8⁺ T cells and almost no NK cells into the tumor were observed. PD-L1 was expressed mainly on tumor cells. Following treatment with SO-C101, tumor biopsies showed a high level of CD8⁺ T cell infiltration, a robustly increased PD-L1 expression on malignant as well as immune cells, and increased NK cell levels (see Table 4 and FIG. 2 F to M).

Accordingly, under treatment with SO-C101 the tumor changed from an only moderately immune cell-infiltrated tumor, which was responsive to SO-C101 treatment as documented by the observed partial response, into a highly immune cell-infiltrated “hot” tumor showing strong PD-L1 checkpoint expression. This also suggests an acquired resistance to SO-C101 treatment. The initial low expression of PD-L1 seems to provide an explanation of the patient's weak response to the earlier treatment with Cemiplimab (anti-PD-1 antibody) showing rather limited success.

The inventors conclude that the induction of PD-L1 expression on tumor cells caused by the treatment with an IL-2/IL-15βγ agonist (re-)sensitized the tumor for (another) treatment with an immune checkpoint inhibitor, here the anti-PD-1 antibody pembrolizumab.

3. Case Report of Patient with Thyroid Gland Carcinoma A 47-year old female patient (race and ethnicity not reported) with thyroid gland carcinoma, was treated s.c. with SO-C101 at 3 μg/kg in combination with 200 mg pembrolizumab within the clinical study SC 103 part B (example 1) starting with the first dose on Nov. 20, 2020.

In medical history there were multiple surgeries from 2008 to 2009 with a partial thyroidectomy and subsequent total thyroidectomy including left cervical lymphadenectomy. In 2017 a liver lesion was treated by radiotherapy. The patient received with the kinase inhibitor vandentanib from 2014 to 2018 one line of previous systemic anticancer therapy. The last disease progression was of documented on July 2020.

Prior to initiation of the treatment, the target lesion in liver segment II had a diameter of 22 mm (CT scan), with two further non-target lesions in liver and bone. Tumor staging on Dec. 29, 2020 (diameter of 25 mm, +13%) and Feb. 11, 2021 (diameter of 18 mm, −18%) showed stable disease, that on Mar. 5, 2021, after 6 cycles of treatment, turned into a partial response (diameter of 15 mm, −31%), which was confirmed on May 5 after 8 cycles (diameter of 14 mm, −36%). On Jul. 21, 2021 treatment was still continuing after 10 cycles of treatment.

Infiltration of immune cells into the tumor was determined by immuno-histochemistry on tumor biopsies obtained at baseline and 6 weeks after SO-C101 treatment as described in Example 2.

TABLE 5
Infiltration of immune cells

| | CD8⁺ T cells | PD-L1⁺ cells | NKp46⁺ NK cells |
|---|---|---|---|
| | [cells/mm²] | [cells/mm²] | [cells/mm²] |
| baseline | 2 | 0 | 0 |
| Week 6 | 20 | 0 | 9 |
| increase | ~10fold | no change | large |

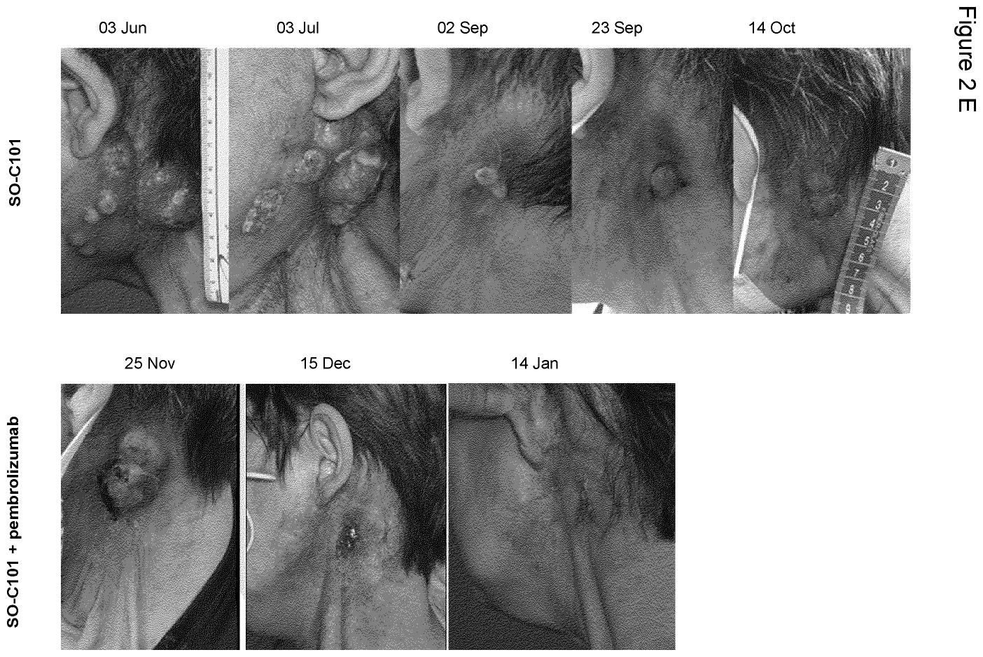

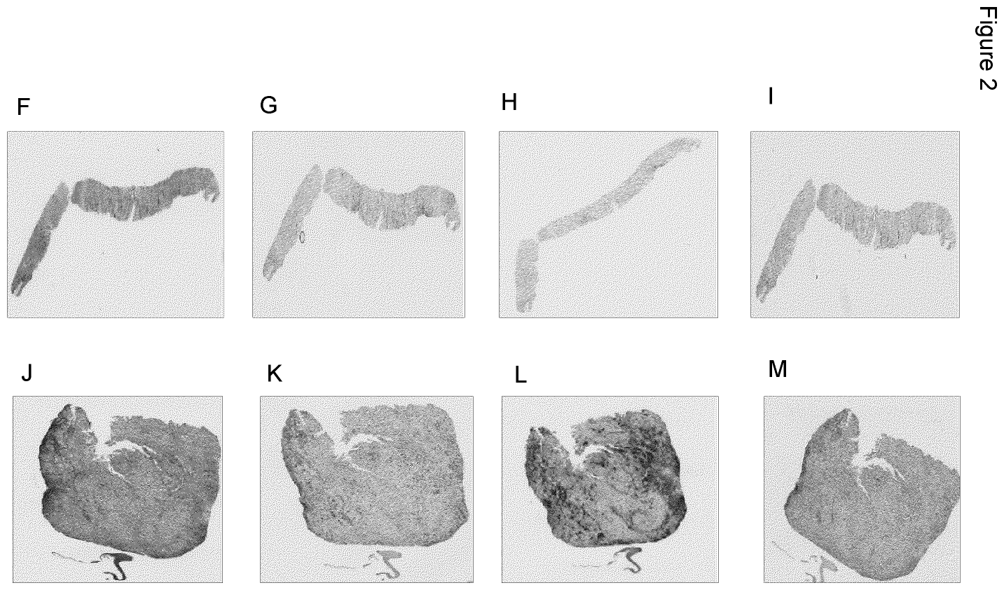

Prior to SO-C101 and pembrolizumab treatment the stage of the tumor can be described as a “cold” tumor due to hardly any infiltration by CD8⁺ T cells and NK cells in the tumor microenvironment. Following the treatment with SC-101 and pembrolizumab, about 10fold more CD8⁺ T cells were found accumulated in the stroma and also scattered throughout the tumor nest. Infiltrated NK cells were scattered throughout the intra-tumoral stroma and also tumor nests. Interestingly, under the co-treatment with pembrolizumab, an increased expression of PD-L1 on tumor cells was not observed. (see Table 5, FIG. 3)

4. Case Report of Patient with Skin Squamous Cell Carcinoma

A 74-year old female patient (race and ethnicity not reported) with skin squamous cell carcinoma (SSCC) of the left leg was treated s.c. with SO-C101 at 6 μg/kg in combination with 200 mg pembrolizumab q3w within the clinical study SC 103 part B (example 1) starting with the first dose on Mar. 11, 2021.

In medical history, SSCC was initially diagnosed in 2006, followed by multiple surgeries, in total 22. From Nov. 6, 2020 to Jan. 29, 2021 the patient received four infusions of the anti-PD-1 antibody cemiplimab with no market response. Therefore, the patient was deemed to be primary resistant to anti-PD-1 therapy.

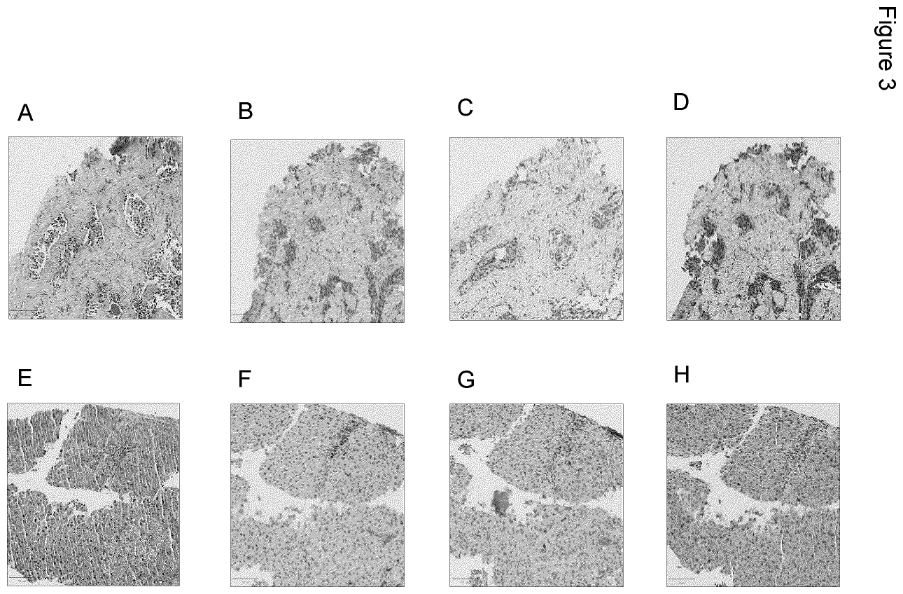

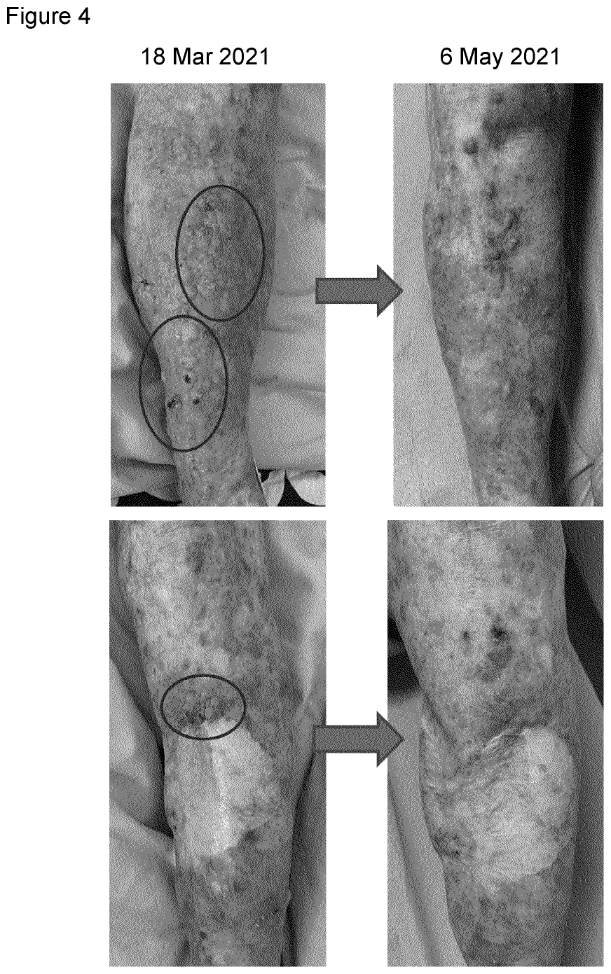

Combination therapy with SO-CIO at 6 μg/kg and 200 mg pembrolizumab started on 11 Mar. 2021. A partial response was observed after two cycles visual on photographs (see FIG. 4) or CT scan, where a decrease of the target lesions was below −39%, which was again confirmed after cycle 4 (CT scan). Treatment still continues after 8 cycles.

Accordingly, despite the relatively small number of patients in this phase I, already two patients with advanced SSCC resistant/refractory to treatment with an anti-PD-1 antibody, showed clear responses to the treatment with SO-C101 alone or in combination with an anti-PD-1 antibody.

5. Case Report of Patient with Cervical Adenocarcinoma

A 63-year old female patient (race and ethnicity not reported) with cervical adenocarcinoma was treated s.c. with SO-C101 at 6 μg/kg in combination with 200 mg pembrolizumab q3w within the clinical study SC 103 part B (example 1) starting May 27, 2021.

In medical history, cervical adenocarcinoma was diagnosed in 2017, followed by radiotherapy, Brachytherapy and surgeries. Systemic chemotherapy with carboplatin from June 2017 to August 2017 was followed by the combination of carboplatin and paclitaxel from March 2018 to June 2018. In 3^{rd} line the patient received cabozantinib from July 2020 to November 2020. The last disease progression was documented on Mar. 29, 2021.

Combination therapy with SO-C101 at 6 μg/kg and 200 mg pembrolizumab started on 27 May 2021. Stable disease was observed for the first and second post-baseline assessments. Cycle 4 was started on 29 Jul. 2021 and treatment still continues.

6. Case Report of Patient with Anus Carcinoma

A 49-year old female patient with anal squamous cell carcinoma, who was refractory after two prior lines of therapy, most recent treatment was with Retifanlimab (anti-PD-1 immune checkpoint inhibitory) treatment from November 2019 until April 2020. The patient was treated starting May 9, 2020 with the combination of 1.5 μg/kg SO-C101 with 200 mg pembrolizumab Q3W. A long-term stable disease of about 48 weeks was observed upon SO-C101 and pembrolizumab therapy and treatment was discontinued due to progressive disease after 18 cycles of treatment. The best response was observed after 8 cycles with a 9% tumor size reduction.

Infiltration of immune cells into the tumor was determined by immuno-histochemistry on tumor biopsies obtained at baseline and 6 weeks after SO-C101 treatment as described in Example 2.

TABLE 6
Infiltration of immune cells

| | CD8⁺ T cells | PD-L1⁺ cells | NKp46⁺ NK cells |
|---|---|---|---|
| | [cells/mm²] | [cells/mm²] | [cells/mm²] |
| baseline | 753 | 537 | 0 |
| Week 6 | 1586 | 1863 | 40 |
| increase | ~2fold | ~3.5fold | large |

The patient presented with a “hot” tumor microenvironment prior to SO-C101 and pembrolizumab treatment characterized by a high infiltration with CD8⁺ T cells and high intra-tumoral density of PD-L1⁺ cells. Following treatment with SO-C101 and pembrolizumab, a further marked increase of infiltration by CD8⁺ T cells and PD-L1⁺ cells was observed in stroma as well as tumor nests. Newly infiltrated NK cells were scattered throughout the intra-tumoral stroma and tumor nest (see Table 6).

7. Pharmacodynamic Responses and Anti-Tumor Immune Activation in Clinical Trial with SO-C101

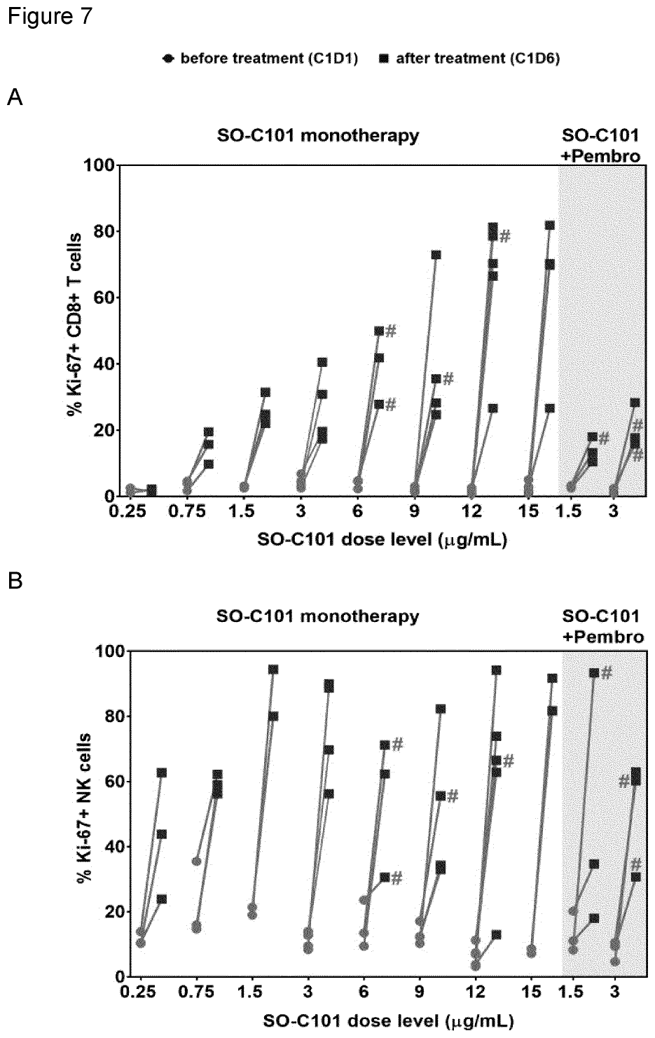

PBMCs were obtained from 26 patients treated with SO-C101 monotherapy and 6 patients treated with SO-C101 and pembrolizumab before treatment on day 1, cycle 1 (CID1) and after treatment on day 6, cycle 1 (C1D6). Percentage of Ki-67⁺ cells within CD8⁺ T cells and (B) NK cells was analyzed by flow cytometry. Increased proliferation of CD8⁺ T cells and NK cells was observed for all patients following treatment with SO-C101 and SO-C101 and pembrolizumab in peripheral blood. Increases were dose dependent for CD8⁺ T cells over the full range from 0.25 until 12 μg/kg, whereas NK cell activation seems to have reached a plateau already at about 1.5 μg/kg. Clinically response patients having either a partial response or at least stable disease over two tumor assessments (marked with #) did not show marked differences for the immune cell activation in blood compared to non-responsive patients (see FIG. 7).

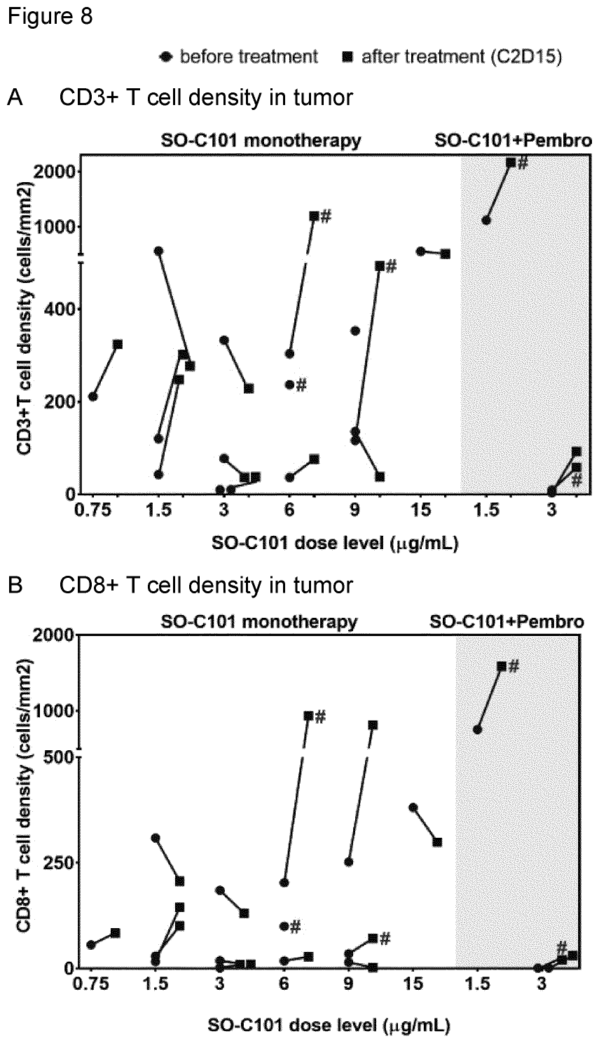

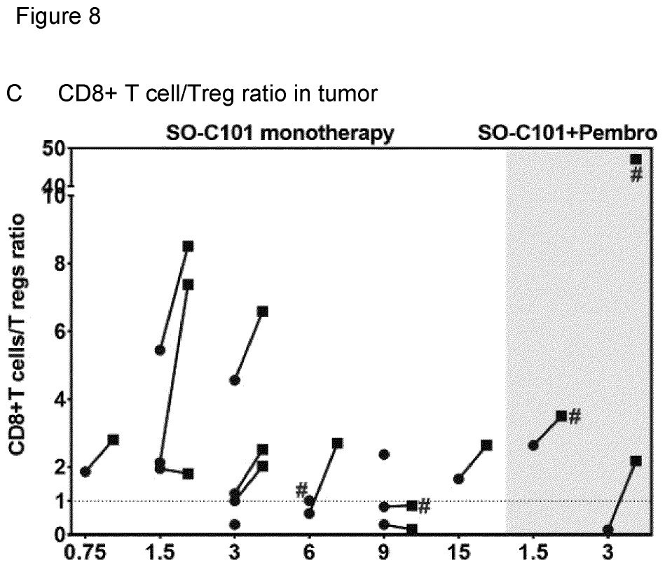

Tumor biopsies were taken at baseline and after treatment (Cycle 2, day 15; C2D15) from 18 patients (15 treated with SO-C101 monotherapy, 3 with SO-C101 and pembrolizumab) and were subjected to immunohistochemistry (IHC) analysis according to standard protocols. Enhanced infiltration of CD3⁺ T cells was observed in 9 out of 18 patients (50%) (FIG. 8 A), enhanced infiltration of CD8⁺ T cells in 9 out of 18 patients (50%) (FIG. 8 B) and increased CD8⁺ T cell/T_{reg} ratio in 10 out of 18 patients (55%) (FIG. 8 C). Clinically responsive patients (PR or ≥2SD, marked with #) showed increased density of CD3⁺ and CD8⁺ T cells as well as an increased ratio of CD8⁺ T cells to T_{regs} in the tumor tissue, whereas non-responsive patients showed a highly heterogenous picture with some increases, some declines in immune cell infiltration.

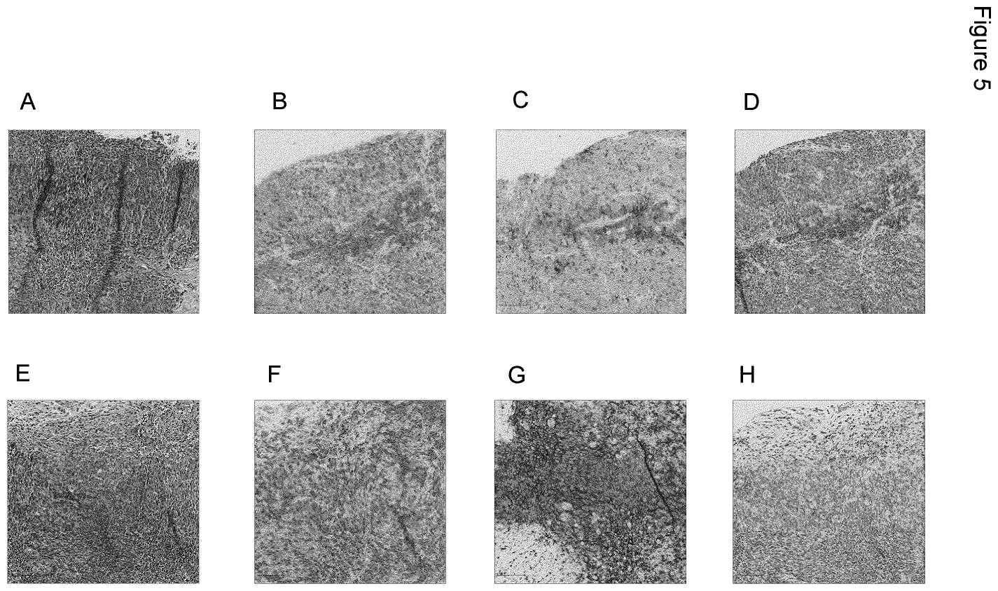

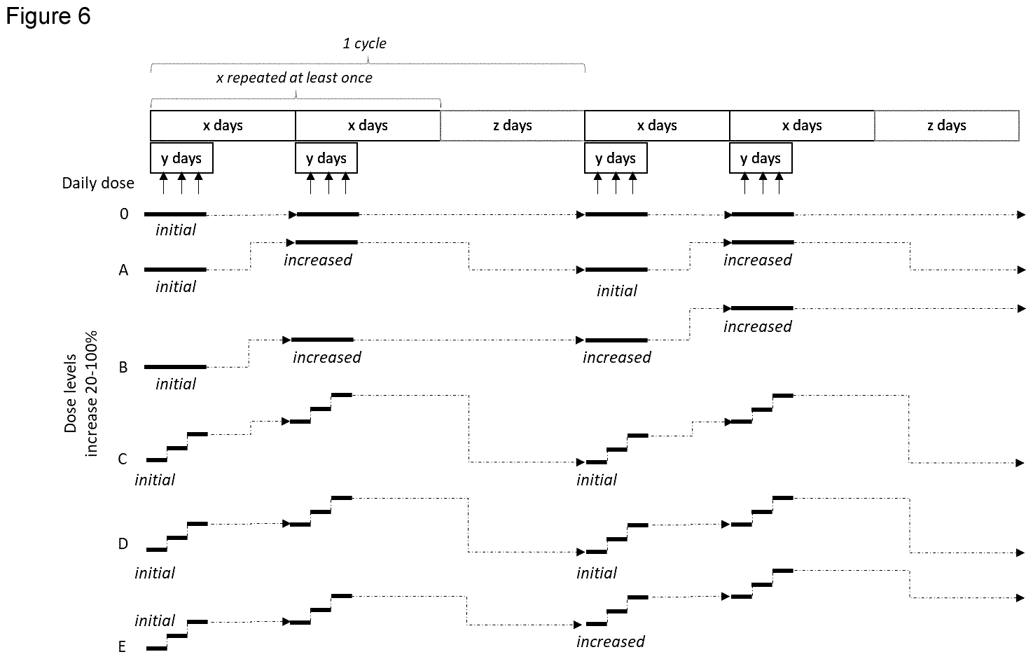

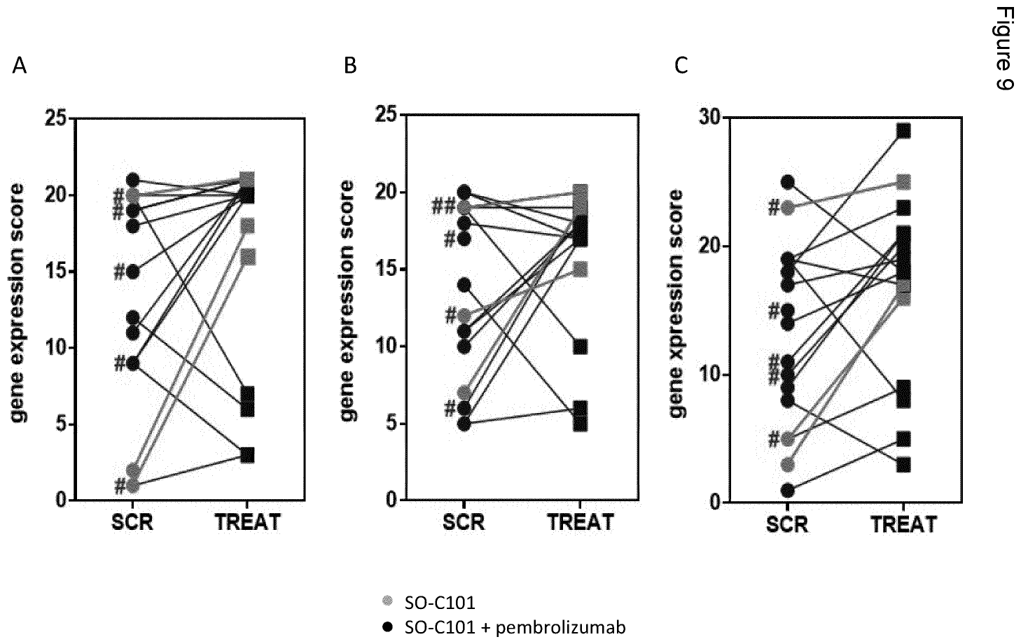

NanoString profiling of tumor tissues from SO-C101 treated patients was performed by HalioDX. NanoString analysis was performed on matched screening and on-treatment (cycle 2 day 15) biopsies. SO-C101 increased the pre-defined set of the HalioDX Immunosign® 21 gene signature score reflecting T cell activation, attraction, cytotoxicity and T cell orientation in 11 out of 18 patients (61%, see FIG. 9 A). SO-C101 also increased the expression of genes linked to antigen processing and presentation in 11 out of 18 patients (61%, see FIG. 9 B). And SO-C101 increased the expression of genes linked to NK cell functions in 13 out of 18 patients (72%, see FIG. 9 C). Robust immune cell infiltration in clinically responsive patients was further visually observed in patients described above (see FIG. 2 F to M, FIG. 3 A to H, and FIG. 5 A to H).

It appears that the activation of immune cells as measured in the blood is a poor marker for response to the treatment of IL-2/IL-15Rβγ agonists, whereas an increased infiltration of effector immune cells into the tumor is a requirement, but not sufficient in all patients for mounting a clinical response. Clinically responsive patients showed a high induction of genes involved in T cell activation, attraction, cytotoxicity and T cell orientation, antigen processing and NK cell function.

### LITERATURE

Abramson, H. N. (2018). “Monoclonal Antibodies for the Treatment of Multiple Myeloma: An Update.” Int J Mol Sci 19(12).
Arenas-Ramirez, N., et al. (2016). “Improved cancer immunotherapy by a CD25-mimobody conferring selectivity to human interleukin-2.” Sci Transl Med 8(367): 367ra166.
Augustin, J. G., et al. (2020). “HPV Detection in Head and Neck Squamous Cell Carcinomas: What Is the Issue?” Front Oncol 10: 1751.
Bacac, M., et al. (2017). “Abstract 1594: Enhancement of the anti-tumor activity of CEA TCB via combination with checkpoint blockade by PD-L1 and interleukin-2 variant immunocytokine.” Cancer Research 77(13 Supplement): 1594.
Bacac, M., et al. (2016). “A Novel Carcinoembryonic Antigen T-Cell Bispecific Antibody (CEA TCB) for the Treatment of Solid Tumors.” Clin Cancer Res 22(13): 3286-3297.
Bentebibel, S. E., et al. (2017). The Novel IL-2 Cytokine Immune Agonist NKTR-214 Harnesses the Adaptive and Innate Immune System for the Treatment of Solid Cancers. Society for Immunotherapy of Cancer 2017 Annual Meeting. National Harbor, MD.
Bergamaschi, C., et al. (2018). “Optimized administration of hetIL-15 expands lymphocytes and minimizes toxicity in rhesus macaques.” Cytokine 108: 213-224.
Bernett, M. J., et al. (2017). “IL15/IL15Rα heterodimeric Fc-fusions with extended half-lives.” Proceedings of the American Association for Cancer Research 58: 408.
Bouda, M., et al. (2000). “High risk” HPV types are frequently detected in potentially malignant and malignant oral lesions, but not in normal oral mucosa.” Mod Pathol 13(6): 644-653.
Caffaro, C. E., et al. (2019). Discovery of pharmacologically differentiated Interleukin 15 (IL-15) agonists employing a synthetic biology platform. SITC 2019. National Harbor, Maryland.
Castro, I., et al. (2011). “The basis of distinctive IL-2- and IL-15-dependent signaling: weak CD122-dependent signaling favors CD8+ T central-memory cell survival but not T effector-memory cell development.” J Immunol 187(10): 5170-5182.
Charych, D., et al. (2017). “Modeling the receptor pharmacology, pharmacokinetics, and pharmacodynamics of NKTR-214, a kinetically-controlled interleukin-2 (IL2) receptor agonist for cancer immunotherapy.” PLoS One 12(7): e0179431.
Charych, D., et al. (2013). “Abstract 482: Tipping the balance in the tumor microenvironment: An engineered cytokine (NKTR-214) with altered IL2 receptor binding selectivity and improved efficacy.” Cancer Research 73(8 Supplement): 482.
Charych, D. H., et al. (2016). “NKTR-214, an Engineered Cytokine with Biased IL2 Receptor Binding, Increased Tumor Exposure, and Marked Efficacy in Mouse Tumor Models.” Clinical Cancer Research 22(3): 680.
Chenoweth, M. J., et al. (2012). “IL-15 can signal via IL-15Ralpha, JNK, and NF-kappaB to drive RANTES production by myeloid cells.” J Immunol 188(9): 4149-4157.
Conlon, K., et al. (2019). Phase I/Ib study of NIZ985 with and without spartalizumab (PDR001) in patients with metastatic/unresectable solid tumors. AACR Annual Meeting, Atlanta, GA.
Conlon, K. C., et al. (2015). “Redistribution, hyperproliferation, activation of natural killer cells and CD8 T cells, and cytokine production during first-in-human clinical trial of recombinant human interleukin-15 in patients with cancer.” J Clin Oncol 33(1): 74-82.
Conlon, K. C., et al. (2019). “Cytokines in the Treatment of Cancer.” J Interferon Cytokine Res 39(1): 6-21.
Darvin, P., et al. (2018). “Immune checkpoint inhibitors: recent progress and potential biomarkers.” Exp Mol Med 50(12): 165.
De Sousa Linhares, A., et al. (2018). “Not All Immune Checkpoints Are Created Equal.” Frontiers in Immunology 9(1909).
Edgar, R. C. (2004). “MUSCLE: multiple sequence alignment with high accuracy and high throughput.” Nucleic Acids Res 32(5): 1792-1797.
Elpek, K. G., et al. (2010). “Mature natural killer cells with phenotypic and functional alterations accumulate upon sustained stimulation with IL-15/IL-15Ralpha complexes.” Proc Natl Acad Sci USA 107(50): 21647-21652.
Felices, M., et al. (2018). “Continuous treatment with IL-15 exhausts human NK cells via a metabolic defect.” JCI Insight 3(3).
Frutoso, M., et al. (2018). “Emergence of NK Cell Hyporesponsiveness after Two IL-15 Stimulation Cycles.” J Immunol 201(2): 493-506.
Fyfe, G., et al. (1995). “Results of treatment of 255 patients with metastatic renal cell carcinoma who received high-dose recombinant interleukin-2 therapy.” J Clin Oncol 13(3): 688-696.
Gajewski, T. F., et al. (2013). “Cancer immunotherapy strategies based on overcoming barriers within the tumor microenvironment.” Curr Opin Immunol 25(2): 268-276.
Gearing, A. J. and R. Thorpe (1988). “The international standard for human interleukin-2. Calibration by international collaborative study.” J Immunol Methods 114(1-2): 3-9.
Ghasemi, R., et al. (2016). “Selective targeting of IL-2 to NKG2D bearing cells for improved immunotherapy.” Nat Commun 7: 12878.
Giron-Michel, J., et al. (2005). “Membrane-bound and soluble IL-15/IL-15Ralpha complexes display differential signaling and functions on human hematopoietic progenitors.” Blood 106(7): 2302-2310.
Goujon, M., et al. (2010). “A new bioinformatics analysis tools framework at EMBL-EBI.” Nucleic Acids Res 38(Web Server issue): W695-699.
Haanen, J. B. (2013). “Immunotherapy of melanoma.” EJC Suppl 11(2): 97-105.
Han, K. P., et al. (2011). “IL-15:IL-15 receptor alpha superagonist complex: high-level co-expression in recombinant mammalian cells, purification and characterization.” Cytokine 56(3): 804-810.
Heaton, K. M., et al. (1993). “Human interleukin 2 analogues that preferentially bind the intermediate-affinity interleukin 2 receptor lead to reduced secondary cytokine secretion: implications for the use of these interleukin 2 analogues in cancer immunotherapy.” Cancer Res 53(11): 2597-2602.
Hon, T., et al. (1987). “Establishment of an interleukin 2-dependent human T cell line from a patient with T cell chronic lymphocytic leukemia who is not infected with human T cell leukemia/lymphoma virus.” Blood 70(4): 1069-1072.
Howley, P. M. and H. J. Pfister (2015). “Beta genus papillomaviruses and skin cancer.” Virology 479-480: 290-296.
Hu, P., et al. (2003). “Generation of low-toxicity interleukin-2 fusion proteins devoid of vasopermeability activity.” Blood 101(12): 4853-4861.
Hu, Q., et al. (2018). “Discovery of a novel IL-15 based protein with improved developability and efficacy for cancer immunotherapy.” Sci Rep 8(1): 7675.
Joseph, I. B., et al. (2019). “THOR-707, a novel not-alpha IL-2, elicits durable pharmacodynamic responses in non-human primates and efficacy as single agent and in combination with anti PD-1 in multiple syngeneic mouse models.” Proceedings of the American Association for Cancer Research 60: 838.
Kinter, A. L., et al. (2008). “The common gamma-chain cytokines IL-2, IL-7, IL-15, and IL-21 induce the expression of programmed death-1 and its ligands.” J Immunol 181(10): 6738-6746.
Klein, C. (2014). “S41. Novel CEA-targeted IL2 variant immunocytokine for immunotherapy of cancer.” Journal for Immunotherapy of Cancer 2(Suppl 2): I8-I8.
Klein, C., et al. (2013). “Abstract PR8: Novel tumor-targeted, engineered IL-2 variant (IL-2v)-based immunocytokines for immunotherapy of cancer.” Cancer Research 73(1 Supplement): PR8.
Klein, C., et al. (2017). “Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines.” Oncoimmunology 6(3): e1277306.
Kurowska, M., et al. (2002). “Fibroblast-like synoviocytes from rheumatoid arthritis patients express functional IL-15 receptor complex: endogenous IL-15 in autocrine fashion enhances cell proliferation and expression of Bcl-x(L) and Bcl-2.” J Immunol 169(4): 1760-1767.
Larsen, S. K., et al. (2014). “NK cells in the tumor microenvironment.” Crit Rev Oncog 19(1-2): 91-105.
Lazear, E., et al. (2017). “Targeting of IL-2 to cytotoxic lymphocytes as an improved method of cytokine-driven immunotherapy.” Oncoimmunology 6(2): e1265721.
Liu, B., et al. (2018). “Evaluation of the biological activities of the IL-15 superagonist complex, ALT-803, following intravenous versus subcutaneous administration in murine models.” Cytokine 107: 105-112.
Lopes, J. E., et al. (2020). “ALKS 4230: a novel engineered IL-2 fusion protein with an improved cellular selectivity profile for cancer immunotherapy.” J Immunother Cancer 8(1).
Margolin, K., et al. (2018). “Phase I Trial of ALT-803, a Novel Recombinant Interleukin-15 Complex, in Patients with Advanced Solid Tumors.” Clin Cancer Res 24(22): 5552-5561.
Miller, J. S., et al. (2018). “A First-in-Human Phase I Study of Subcutaneous Outpatient Recombinant Human IL15 (rhIL15) in Adults with Advanced Solid Tumors.” Clin Cancer Res 24(7): 1525-1535.
Miyazaki, T., et al. (2018). “Pharmacokinetic and Pharmacodynamic Study of NKTR-255, a Polymer-Conjugated Human IL-15, in Cynomolgus Monkey.” Blood 132(Suppl 1): 2952-2952.
Needleman, S. B. and C. D. Wunsch (1970). “A general method applicable to the search for similarities in the amino acid sequence of two proteins.” J Mol Biol 48(3): 443-453.
Paradisi, A., et al. (2020). “Concomitant seropositivity for HPV 16 and cutaneous HPV types increases the risk of recurrent squamous cell carcinoma of the skin.” Eur J Dermatol 30(5): 493-498.
Pearson, W. R. and D. J. Lipman (1988). “Improved tools for biological sequence comparison.” Proc Natl Acad Sci USA 85(8): 2444-2448.
Perdreau, H., et al. (2010). “Different dynamics of IL-15R activation following IL-15 cis- or trans-presentation.” Eur Cytokine Netw 21(4): 297-307.
Prattichizzo, C., et al. (2016). “Establishment and characterization of a highly immunogenic human renal carcinoma cell line.” International journal of oncology 49(2): 457-470.
Rhode, P. R., et al. (2016). “Comparison of the Superagonist Complex, ALT-803, to IL15 as Cancer Immunotherapeutics in Animal Models.” Cancer Immunol Res 4(1): 49-60.
Ring, A. M., et al. (2012). “Mechanistic and structural insight into the functional dichotomy between IL-2 and IL-15.” Nat Immunol 13(12): 1187-1195.
Robinson, T. O. and K. S. Schluns (2017). “The potential and promise of IL-15 in immuno-oncogenic therapies.” Immunol Lett 190: 159-168.
Romee, R., et al. (2018). “First-in-human Phase 1 Clinical Study of the IL-15 Superagonist Complex ALT-803 to Treat Relapse after Transplantation.” Blood 131(23): 2515-2527.
Rosenzwajg, M., et al. (2019). “Immunological and clinical effects of low-dose interleukin-2 across 11 autoimmune diseases in a single, open clinical trial.” Ann Rheum Dis 78(2): 209-217.
Shanafelt, A. B., et al. (2000). “A T-cell-selective interleukin 2 mutein exhibits potent antitumor activity and is well tolerated in vivo.” Nat Biotechnol 18(11): 1197-1202.
Sharma, P., et al. (2017). “Primary, Adaptive, and Acquired Resistance to Cancer Immunotherapy.” Cell 168(4): 707-723.
Silva, D.-A., et al. (2019). “De novo design of potent and selective mimics of IL-2 and IL-15.” Nature 565(7738): 186-191.
Smith, T. F. and M. S. Waterman (1981). “Comparison of biosequences.” Advances in Applied Mathematics 2(4): 482-489.
Smola, S. (2017). “Immunopathogenesis of HPV-Associated Cancers and Prospects for Immunotherapy.” Viruses 9(9).
Solomon, B. L. and I. Garrido-Laguna (2018). “TIGIT: a novel immunotherapy target moving from bench to bedside.” Cancer Immunol Immunother 67(11): 1659-1667.
Soman, G., et al. (2009). “MTS dye based colorimetric CTLL-2 cell proliferation assay for product release and stability monitoring of interleukin-15: assay qualification, standardization and statistical analysis.” J Immunol Methods 348(1-2): 83-94.
Steel, J. C., et al. (2012). “Interleukin-15 biology and its therapeutic implications in cancer.” Trends Pharmacol Sci 33(1): 35-41.
Sterling, J. C. (2005). “Human papillomaviruses and skin cancer.” J Clin Virol 32 Suppl 1: S67-71.
Thaysen-Andersen, M., et al. (2016). “Recombinant human heterodimeric IL-15 complex displays extensive and reproducible N- and O-linked glycosylation.” Glycoconj J 33(3): 417-433.
Toutain, P. L. and A. Bousquet-Melou (2004). “Plasma terminal half-life.” J Vet Pharmacol Ther 27(6): 427-439.
Tumban, E. (2019). “A Current Update on Human Papillomavirus-Associated Head and Neck Cancers.” Viruses 11(10).
Wadhwa, M., et al. (2013). “The 2nd International standard for Interleukin-2 (IL-2) Report of a collaborative study.” Journal of Immunological Methods 397(1): 1-7.
Waldmann, T. A. (2015). “The shared and contrasting roles of IL2 and IL15 in the life and death of normal and neoplastic lymphocytes: implications for cancer therapy.” Cancer Immunol Res 3(3): 219-227.
Wei, X., et al. (2001). “The Sushi domain of soluble IL-15 receptor alpha is essential for binding IL-15 and inhibiting inflammatory and allogenic responses in vitro and in vivo.” J Immunol 167(1): 277-282.
Wrangle, J. M., et al. (2018). “ALT-803, an IL-15 superagonist, in combination with nivolumab in patients with metastatic non-small cell lung cancer: a non-randomised, open-label, phase 1b trial.” Lancet Oncol 19(5): 694-704.
WO 2005/085282A1
WO 2007/046006A2
WO 2008/003473A2
WO 2008/143794A1
WO 2009/135031A1
WO 2012/065086A1
WO 2012/107417A1
WO 2012/175222A1
WO 2014/066527A2
WO 2014/145806A2
WO 2014/207173A1
WO 2015/018528A1
WO 2015/109124A2
WO 2016/060996A2
WO 2016/095642
WO 2016/142314A1
WO 2017/046200A1
WO 2017/112528A2
WO 2018/071918A1
WO 2018/071919A1
WO 2018/102536A1
WO 2018/151868A2
WO 2018/213341A1
WO 2019/028419A1
WO 2019/028425A1
WO 2019/165453A1
WO 2019/173798A1
WO 2020/069398A1
WO 2020/232305A1
WO 2021/081193A1
WO 2021/156720A1
US 2003/0124678
US 2006/0057680
US 2007/0036752
US 2017/0088597
US 2018/0118805
US 2019/0092830
U.S. Pat. No. 5,229,109
U.S. Pat. No. 10,206,980
NCT02983045 at www.clinicaltials.gov, as of 16 Aug. 2018
NCT03386721 at www.clinicaltrials.gov, as of 16 Aug. 2018
NCT02627284 at www.clinicaltnals.gov, as of 16 Aug. 2018
NCT03063762 at www.clinicaltrails.gov, as of 16 Aug. 2018
NCT03388632, at www.cinicaltrials.gov, as of 16 Aug. 2018
NCT01572493, at www.clinicaltrials.gov, as of 16 Aug. 2018
NCT01021059, at www.clinicaltrials.gov. as of 14 May 2019

1. An interleukin 15 (IL-15)/interleukin-15 receptor alpha (IL-15Rα) complex for use in the treatment of squamous cell carcinoma in a human patient, wherein the patient is resistant or refractory to at least one immune checkpoint inhibitor treatment.

2. The IL-15/IL-15Rα complex for the use of claim 1, whereas the squamous cell carcinoma is selected from the group consisting of skin squamous cell carcinoma, non-small-cell lung carcinoma (NSCLC), especially squamous-cell carcinoma of the lung (SCC), squamous cell thyroid carcinoma, head and neck squamous cell carcinoma (HNSCC), oral squamous cell carcinoma, oropharyngeal squamous cell carcinoma, and laryngeal squamous cell carcinoma, esophageal squamous cell carcinoma, esophageal and gastro-esophageal junction cancer squamous cell carcinoma, vaginal squamous-cell carcinoma, penile squamous cell carcinoma, anal squamous cell carcinoma, prostate squamous cell carcinoma, and bladder squamous cell carcinoma, especially skin squamous cell carcinoma.

3. (canceled)

4. The IL-15/IL-15Rα complex for the use of claim 1, wherein the IL-15/IL-15Rα complex is not administered in combination with an immune checkpoint inhibitor.

5. The IL-15/IL-15Rα complex for the use of claim 1, wherein the IL-15/IL-15Rα complex is not administered in combination with a PD-1 antagonist.

6. The IL-15/IL-15Rα complex for the use of claim 1, wherein the IL-15/IL-15Rα complex is not administered in combination with the immune checkpoint inhibitor the patient is refractory or resistant to, preferably wherein the immune checkpoint inhibitor the patient is refractory or resistant to and that is not administered in combination is a PD-1 antagonist.

7. The IL-15/IL-15Rα complex for the use of claim 1, wherein the IL-15/IL-15Rα complex is administered in combination with an immune checkpoint inhibitor.

8. The IL-15/IL-15Rα complex for the use of claim 7, wherein the IL-15/IL-15Rα complex is administered in combination with a PD-1 antagonist.

9. The IL-15/IL-15Rα complex for the use of claim 7, wherein the IL-15/IL-15Rα complex is administered in combination with the immune checkpoint inhibitor the patient is refractory or resistant to, preferably wherein the immune checkpoint inhibitor the patient is refractory or resistant to and that is administered in combination is a PD-1 antagonist.

10. The IL-15/IL-15Rα complex for the use of claim 9, wherein the treatment of the cancer results in at least about 30% size reduction of the tumor present prior to the treatment, preferably about 30% size reduction within 16 weeks of the treatment, preferably about 50% size reduction within 16 weeks of the treatment.

11. The IL-15/IL-15Rα complex for the use of any claim 10, wherein the response to the IL-15/IL-15Rα complex is mediated by the innate immune response mediated by NK cells.

12. The IL-15/IL-15Rα complex for the use of claim 11, whereas the IL-15/IL-15Rα complex is administered according to a cyclical administration regimen, wherein the cyclical administration regimen comprises:
(a) a first period of x days during which the IL-15/IL-15Rα complex is administered at a daily dose on y consecutive days at the beginning of the first period followed by x-y days without administration of the IL-15/IL-15Rα complex,
wherein x is 5, 6, 7, 8, 9, 10, 11, 12, 13, 14, 15, 16, 17, 18, 19, 20 or 21 days, preferably, 7 or 14 days, and y is 2, 3 or 4 days, preferably 2 or 3 days;
(b) repeating the first period at least once; and
(c) a second period of z days without administration of the IL-15/IL-15Rα complex,
wherein z is 5, 6, 7, 8, 9, 10, 11, 12, 13, 14, 15, 16, 17, 18, 19, 20, 21, 28, 35, 42, 49, 56, 63 or 70 days, preferably 7, 14, 21 or 56 days, more preferably 7, 14 or 21 days.

13. The IL-15/IL-15Rα complex for the use of claim 12, wherein x is 7 days, y is 2, 3 or 4 days and z is 7 days, preferably wherein y is 2 days and z is 7 days.

14. The IL-15/IL-15Rα complex for the use of claim 13, wherein the daily dose of the IL-15/IL-15Rα complex is 0.1 μg/kg to 50 μg/kg, preferably 0.25 μg/kg to 25 μg/kg, more preferably 0.6 μg/kg to 12 μg/kg and even more preferably 2 μg/kg to 12 μg/kg, preferably 3 μg/kg to 20 μg/kg, more preferably 6 to 12 μg/kg.

15. The IL-15/IL-15Rα complex for the use of claim 14, wherein the IL-15/IL-15Rα complex a fusion protein comprising the human IL-15Rα sushi domain or derivative thereof, a flexible linker and the human IL-15 or derivative thereof, preferably wherein the human IL-15Rα sushi domain comprises the sequence of SEQ ID NO: 6, and wherein the human IL-15 comprises the sequence of SEQ ID NO: 4, more preferably wherein the IL-15/IL-15Rα complex is SEQ ID NO: 9.


SEQUENCE LISTING (19 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 6 - protein, 61 aa, Homo sapiens
CPPPMSVEHADIWVKSYSLYSRERYICNSGFKRKAGTSSLTECVLNKATNVAHWTTPSLKC

SEQ ID NO: 7 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 8 - protein, 20 aa, Artificial Sequence
  Linker Sequence
SGGSGGGGSGGGSGGGGSGG

SEQ ID NO: 9 - protein, 211 aa, Artificial Sequence
  RLI2
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 11 - protein, 20 aa, Artificial Sequence
  Leader peptide of (IL-15N72D)2IL-15Ra sushi-Fc
METDTLLLWVLLLWVPGSTG

SEQ ID NO: 12 - protein, 297 aa, Artificial Sequence
  IL-15Ra sushi (65aa)-Fc (IgG1 CH2-CH3)
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 114 aa, Artificial Sequence
  IL-15N72D
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 14 - protein, 205 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 15 - protein, 114 aa, Artificial Sequence
  IL-15 L52C
NWVNVISDLKKIEDLIQSMHIDATLYTESDVHPSCKVTAMKCFLLELQVISCESGDASIHDTVENLIILANNSLSSNGNVTESGCKECEELEEKNIKEFLQSFVHIVQMFINTS

SEQ ID NO: 16 - protein, 315 aa, Artificial Sequence
  IL-15Ra-sushi+S40C-Fc
ITCPPPMSVEHADIWVKSYSLYSRERYICNSGFKRKAGTCSLTECVLNKATNVAHWTTPSLKCIRDPALVHQRGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 100 aa, Artificial Sequence
  NEO 2-15 E62C
PKKKIQLHAEHALYDALMILNIVKTNSPPAEEKLEDYAFNFELILEEIARLFESGDQKDEACKAKRMKEWMKRIKTTASEDEQEEMANAIITILQSWIFS

SEQ ID NO: 18 - protein, 350 aa, Artificial Sequence
  XENP024306 chain 1 human IL-15 D30N/E64Q/N65D (GGGGS)1-Fc(216)_IgG1_pI(-) Isosteric A C2205/PVA_/S267K/L368D/K370S/M428L/N434S
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 19 - protein, 301 aa, Artificial Sequence
  XENP024306 chain 2 human IL15R (sushi) (GGGGS)1-Fc(216)_IgG1_C2205/PVA_/S267K/S364K/E357Q/M428L/N434S
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK